(19) United States
(12) Patent Application Publication
(10) Pub. No.: US 2026/0216197 A1
(43) Pub. Date: Jul. 30, 2026

(54) WRN HELICASE INHIBITOR

(71) Applicant: SUZHOU PUHE BIOPHARMA CO., LTD., Suzhou (CN)

(72) Inventors: Bin Liu, Suzhou (CN); Feng Gao, Suzhou (CN); Yongqi Guo, Suzhou (CN); Yongyong Wu, Suzhou (CN); Liandong Jing, Suzhou (CN); Zhizhong Li, Suzhou (CN); Zhuo Wu, Suzhou (CN)

(21) Appl. No.: 19/143,592

(22) PCT Filed: Dec. 25, 2023
(86) PCT No.: PCT/CN2023/141629

§ 371 Date: Jun. 26, 2025

(30) Foreign Application Priority Data
Dec. 30, 2022 (CN) 202211693494.7
Apr. 25, 2023 (CN) 202310452664.0
May 29, 2023 (CN) 202310617067.9
Jul. 10, 2023 (CN) 202310839129.0
Aug. 31, 2023 (CN) 202311115784.8

(51) Int. Cl. A61K 31/5377; A61K 31/519; A61P 35/00; C07B 59/00; C07D 487/04; C07D 519/00

(52) U.S. Cl. CPC A61K 31/5377; A61K 31/519; A61P 35/00; C07B 59/002; C07D 487/04; C07D 519/00; C07B 2200/05

(57) ABSTRACT

The present invention provides a WRN helicase inhibitor as represented by the compound in formula (I) or a pharmaceutically acceptable salt, isotopic variant, tautomer or stereoisomer thereof.

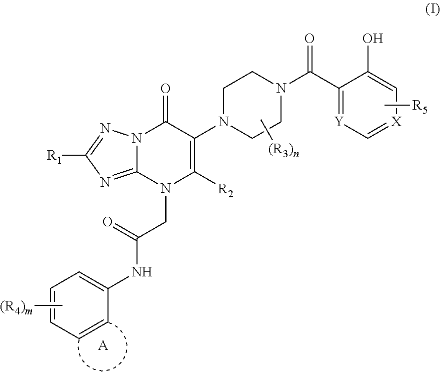

This application claims the priority of:
Chinese application 202211693494.7, filed on Dec. 30, 2022;
Chinese application 202310452664.0, filed on Apr. 25, 2023;
Chinese application 202310617067.9, filed on May 29, 2023;
Chinese application 202310839129.0, filed on Jul. 10, 2023;
Chinese application 202311115784.8, filed on Aug. 31, 2023, which are incorporated herein by reference in their entirety.

### FIELD OF THE INVENTION

The present disclosure belongs to the field of medicine, and in particular relates to a WRN helicase inhibitor.

### BACKGROUND OF THE INVENTION

Abnormal DNA mismatch repair (MMR) can lead to high mutations (deletions or insertions) in the DNA nucleotide repeat region, which is called microsatellite instability (MSI). High microsatellite instability (MSI-H) can induce tumorigenesis, including colorectal cancer, gastric cancer, endometrial cancer, and ovarian cancer, etc. (Nature, 2019, 568, 551-556), with the highest mutation rates observed in colorectal cancer (15%) and gastric cancer (22%). Although immunotherapy PD-1/PD-L1, up to date, has shown excellent therapeutic effects in multiple cancers, for example, pembrolizumab significantly improved median progression-free survival (PFS) compared to chemotherapy in MSI-H advanced colorectal cancer patients and was approved by the FDA as a first-line treatment (N. Engl. J. Med. 2020, 383, 2207-2218), many MSI-H tumor patients still do not benefit from it. In addition, the 2022 ASCO conference reported that in the Phase 2 clinical CheckMate 142 (NCT02060188) trial, using dual immunotherapy (PD-1+CTLA-4: nivolumab+ipilimumab) to treat metastatic colorectal cancer patients, regardless of whether they were in first-line or post-second-line therapy, more than half of the patients still experienced recurrence after a 4-year follow-up. Therefore, there is an urgent need to develop new treatment strategies.

Synthetic lethality means that in tumor cells, the inactivation of either of two genes individually has no significant effect on the survival of tumor cells, but the simultaneous inactivation of both genes leads to the death of tumor cells (Nat. Rev. Drug Discov. 2020, 19(1): 23-38, Cancer Discov. 2021, 11(7):1626-1635). Synthetic lethal targeted drugs can generally produce a good therapeutic safety window, while also increasing the development accessibility of certain targets with high mutation rates but having challenging druggability. Currently, the most successful example of synthetic lethality is PARP1/2 inhibitors, such as Olaparib, Rucaparib, Niraparib, etc, which have demonstrated remarkable efficacy in treating BRCA1/2-mutated ovarian and breast cancers and have been successively approved for clinical use (Nat. Rev. Drug Discov. 2020, 19 (10):711-736; Nat. Rev. Clin. Oncol. 2020, 17(3):136-137). In 2019, Adam J. Bass published consecutive articles in Nature proving that Werner helicase (WRN) is a synthetic lethal target for MSI-H tumors (Nature, 2019, 568, 551-556; Nature, 2019, 586, 292-298). Complete knockout of WRN or introducing a K557M mutation (which abolishes helicase activity) into WRN helicase induced cell cycle arrest and apoptosis in MSI-H tumor cells. Furthermore, Mathew J. Garnett et al. also found that even if the tumor cells of MSI-H patients become resistant after chemotherapy and immunotherapy, WRN inhibition could still further suppress tumor growth (Cancer Discov. 2021, 11, 1923-1937).

Despite the progress in WRN research, there are currently no WRN inhibitors that have entered clinical trials for treating MSI-H-related cancers. The WRN inhibitors with a novel structure provided by the present disclosure are expected to address this unmet clinical need.

### SUMMARY OF THE INVENTION

In one aspect, the present disclosure provides a compound of formula (I), or a pharmaceutically acceptable salt, isotopic variant, tautomer, or stereoisomer thereof:

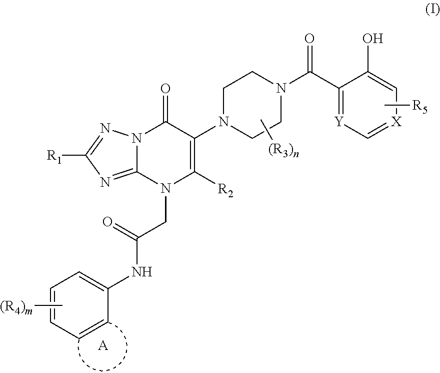

wherein
[symbol] represents a single bond or a double bond;
X and Y are each independently selected from CH and N, and at least one of X and Y is N;
R₁ is selected from

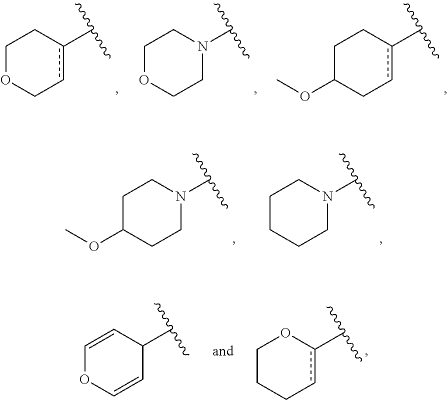

alternatively, R₁ is selected from

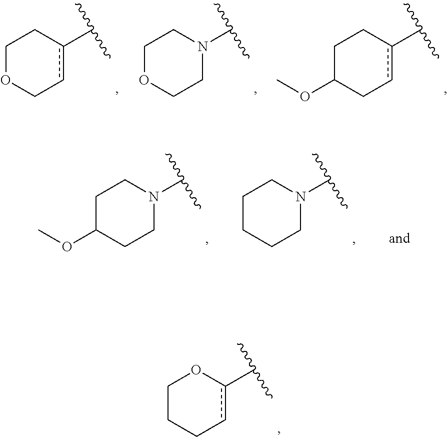

and the R₁ is optionally substituted with 1 or 2 Rₓ;
R₂ is selected from H, C₁₋₆ alkyl, C₁₋₆ haloalkyl, C₁₋₆ alkoxy, C₁₋₆ alkylthio and C₃₋₆ cycloalkyl;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

In another aspect, the present disclosure provides a compound of formula (II), or a pharmaceutically acceptable salt, isotopic variant, tautomer, or stereoisomer thereof:

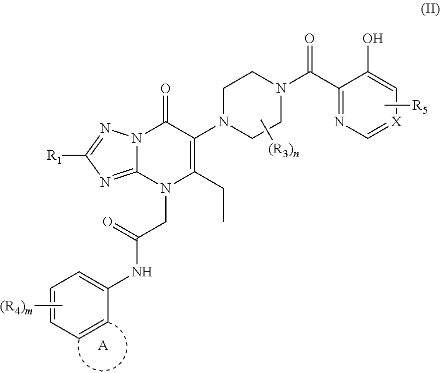

wherein
[symbol] represents a single bond or a double bond;
X is selected from CH and N;
R₁ is selected from

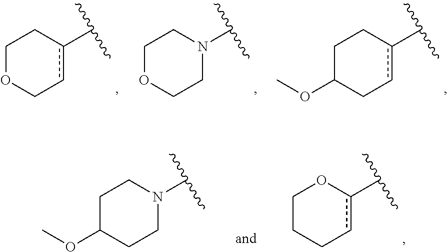

and the R₁ is optionally substituted with 1 or 2 Rₓ;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

In another aspect, the present disclosure provides a pharmaceutical composition comprising a compound of the present disclosure, and optionally a pharmaceutically acceptable excipient.

In another aspect, the present disclosure provides a pharmaceutical composition comprising a compound of the present disclosure and a pharmaceutically acceptable excipient, which also contains other therapeutic agents.

In another aspect, the present disclosure provides use of the compound of the present disclosure in the manufacture of a medicament for treating and/or preventing a WRN-mediated disease.

In another aspect, the present disclosure provides a method for treating and/or preventing a WRN-mediated disease in a subject, comprising administering to the subject the compound or composition of the present disclosure.

In another aspect, the present disclosure provides a compound of the present disclosure or a composition of the present disclosure, for use in treating and/or preventing a WRN-mediated disease.

In a specific embodiment, the diseases treated in the present disclosure include cancers selected from the group consisting of acoustic neuroma, adenocarcinoma, adrenal gland cancer, anal cancer; angiosarcoma (e.g., lymphangiosarcoma, lymphangioendotheliosarcoma, hemangiosarcoma), appendix cancer, benign monoclonal gammopathy, bile duct cancer, bladder cancer, brain cancer (e.g., meningioma, glioma, e.g., astrocytoma, oligodendroglioma, medulloblastoma), bronchial cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer))), hematopoietic cancer (e.g., leukemia, such as acute lymphocytic leukemia (ALL) (e.g., B-cell ALL, T-cell ALL), acute myeloid leukemia (AML) (e.g., B-cell AML, T-cell AML), chronic myeloid leukemia (CML) (e.g., B-cell CML, T-cell CML), chronic lymphocytic leukemia (CLL) (e.g., B-cell CLL, T-cell CLL), follicular lymphoma, chronic lymphocytic leukemia/small lymphocytic lymphoma (CLL/SLL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, lymphoplasmacytic lymphoma, hairy cell leukemia (HCL), immunoblastic large cell lymphoma, precursor B lymphoblastic lymphoma, and primary central nervous system (CNS) lymphomas; and T-cell non-Hodgkin's lymphomas, such as precursor T-lymphoblastic lymphoma/leukemia, peripheral T-cell lymphomas (such as cutaneous T-cell lymphomas (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma (MM), heavy chain disease (e.g., alpha chain disease, gamma chain disease, mu chain disease), hemangioblastoma, inflammatory myofibroblastic tumor, immunocytic amyloidosis, kidney cancer (e.g., nephroblastoma, renal cell carcinoma), liver cancer (e.g., hepatocellular cancer, malignant hepatoma), lung cancer (such as bronchogenic carcinoma, small cell lung cancer (SCLC), non-small cell lung cancer (NSCLC), lung adenocarcinoma, leiomyosarcoma (LMS), mastocytosis (such as systemic mastocytosis), myelodysplastic syndrome (MDS), mesothelioma, myeloproliferative diseases (MPD) (such as polycythemia vera (PV), essential thrombocythemia (ET), agnogenic myeloid metaplasia (AMM), chronic idiopathic myelofibrosis, chronic myeloid leukemia (CML), chronic neutrophilic leukemia (CNL), hypereosinophilic syndrome (HES), neuroblastoma, neurofibromas (such as neurofibromatosis type 1 or type 2, schwannomatosis), neuroendocrine cancer (such as gastroenteropancreatic neuroendocrine tumor (GEP-NET), carcinoid tumor), osteosarcoma, ovarian cancer (such as cystadenocarcinoma, ovarian embryonal carcinoma, ovarian adenocarcinoma), papillary adenocarcinoma, penile cancer.

Other objects and advantages of the present disclosure will be apparent to those skilled in the art from the following detailed description, examples and claims.

### Definition

### Chemical Definitions

Definitions of specific functional groups and chemical terms are described in more detail below.

When a range of values is listed, it is intended to encompass each value and sub-range within the range. For example, “C₁₋₆ alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₆ alkyl” refers to a radical of a straight or branched, saturated hydrocarbon group having 1 to 6 carbon atoms. In some embodiments, C₁₋₄ alkyl and C₁₋₂ alkyl are alternative. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), iPr (—CH(CH₃)₂), nPr (—CH₂CH₂CH₃), n-Bu (—CH₂CH₂CH₂CH₃) or i-Bu (—CH₂CH(CH₃)₂).

“C₂₋₆ alkenyl” refers to a radical of a straight or branched hydrocarbon group having 2 to 6 carbon atoms and at least one carbon-carbon double bond. In some embodiments, C₂₋₄ alkenyl is alternative. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, phosphorus). The alkenyl groups can be optionally substituted with one or more substituents, for example, with 1 to 5 substituents, 1 to 3 substituents or 1 substituent.

“C₂₋₆ alkynyl” refers to a radical of a straight or branched hydrocarbon group having 2 to 6 carbon atoms, at least one carbon-carbon triple bond and optionally one or more carbon-carbon double bonds. In some embodiments, C₂₋₄ alkynyl is alternative. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, phosphorus). The alkynyl groups can be substituted with one or more substituents, for example, with 1 to 5 substituents, 1 to 3 substituents or 1 substituent.

“C₁₋₆ alkylene” refers to a divalent group formed by removing another hydrogen of the C₁₋₆ alkyl, and can be substituted or unsubstituted. In some embodiments, C₁₋₄ alkylene, C₂₋₄ alkylene, and C₁₋₃ alkylene are alternative. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), etc.

“C₂₋₆ alkenylene” refers to a C₂₋₆ alkenyl group wherein another hydrogen is removed to provide a divalent radical of alkenylene, and which may be substituted or unsubstituted. In some embodiments, C₂₋₄ alkenylene is yet alternative. Exemplary unsubstituted alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₆ alkynylene” refers to a C₂₋₆ alkynyl group wherein another hydrogen is removed to provide a divalent radical of alkynylene, and which may be substituted or unsubstituted. In some embodiments, C₂₋₄ alkynylene is yet alternative. Exemplary alkynylene groups include, but are not limited to, ethynylene (—C≡C—), substituted or unsubstituted propynylene (—C≡CCH₂—), and the like.

“Halo” or “halogen” refers to fluorine (F), chlorine (C₁), bromine (Br) and iodine (I).

Thus, “C₁₋₆ haloalkyl” refers to the above “C₁₋₆ alkyl”, which is substituted by one or more halogen. In some embodiments, C₁₋₄ haloalkyl is yet alternative, and still alternatively C₁₋₂ haloalkyl. Exemplary haloalkyl groups include, but are not limited to, —CF₃, —CH₂F, —CHF₂, —CHFCH₂F, —CH₂CHF₂, —CF₂CF₃, —CCl₃, —CH₂Cl, —CHCl₂, 2,2,2-trifluoro-1,1-dimethyl-ethyl, and the like. The haloalkyl can be substituted at any available point of attachment, for example, with 1 to 5 substituents, 1 to 3 substituents or 1 substituent.

“C₁₋₆ alkoxy” refers to a —OR group wherein R is a C₁₋₆ alkyl group as defined above. C₁₋₄ alkoxy is alternative.

“C₁₋₆ haloalkoxy” refers to a “C₁₋₆ alkoxy”, which is substituted with one or more halogen groups. In some embodiments, C₁₋₄haloalkoxy is yet alternative, and C₁₋₂haloalkoxy is still alternative.

“C₃₋₁₀ cycloalkyl” refers to a radical of a non-aromatic cyclic hydrocarbon group having from 3 to 10 ring carbon atoms and zero heteroatoms. In some embodiments, C₄₋₁₀ cycloalkyl, C₅₋₁₀ cycloalkyl, C₄₋₇ cycloalkyl, C₃₋₇ cycloalkyl, C₃₋₆ cycloalkyl, C₃₋₅ cycloalkyl, and C₃₋₄ cycloalkyl are yet alternative, and still alternatively C₅₋₆ cycloalkyl. The cycloalkyl also includes a ring system in which the cycloalkyl described herein is fused with one or more aryl or heteroaryl groups, wherein the point of attachment is on the cycloalkyl ring, and in such case, the number of carbon atoms continues to represent the number of carbon atoms in the cycloalkyl system. Exemplary cycloalkyl groups include, but are not limited to, cyclopropyl (C₃), cyclopropenyl (C₃), cyclobutyl (C₄), cyclobutenyl (C₄), cyclopentyl (C), cyclopentenyl (C), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl can be substituted with one or more substituents, for example, with 1 to 5 substituents, 1 to 3 substituents or 1 substituent.

“3- to 12-membered heterocyclyl” refers to a radical of 3- to 12-membered non-aromatic ring system having ring carbon atoms and 1 to 5 ring heteroatoms, wherein each of the heteroatoms is independently selected from nitrogen, oxygen, sulfur, boron, phosphorus and silicon. In the heterocyclyl containing one or more nitrogen atoms, the point of attachment can be a carbon or nitrogen atom as long as the valence permits. In some embodiments, 3- to 10-membered heterocyclyl is alternative, which is a radical of 3- to 10-membered non-aromatic ring system having ring carbon atoms and 1 to 3 ring heteroatoms. In some embodiments, 4- to 10-membered heterocyclyl is alternative, which is a radical of 4- to 10-membered non-aromatic ring system having ring carbon atoms and 1 to 4 ring heteroatoms. In some embodiments, 5- to 10-membered heterocyclyl is alternative, which is a radical of 5- to 10-membered non-aromatic ring system having ring carbon atoms and 1 to 5 ring heteroatoms. In some embodiments, 5- to 8-membered heterocyclyl is alternative, which is a radical of 5- to 8-membered non-aromatic ring system having ring carbon atoms and 1 to 5 ring heteroatoms. In some embodiments, 5- to 7-membered heterocyclyl is alternative, which is a radical of 5- to 7-membered non-aromatic ring system having ring carbon atoms and 1 to 4 ring heteroatoms. In some embodiments, 3-to 7-membered heterocyclyl is alternative, which is a radical of 3- to 7-membered non-aromatic ring system having ring carbon atoms and 1 to 4 ring heteroatoms. 3- to 6-membered heterocyclyl is alternative, which is a radical of 3- to 6-membered non-aromatic ring system having ring carbon atoms and 1 to 3 ring heteroatoms. 4- to 7-membered heterocyclyl is alternative, which is a radical of 4- to 7-membered non-aromatic ring system having ring carbon atoms and 1 to 3 ring heteroatoms. 4- to 6-membered heterocyclyl is alternative, which is a radical of 4- to 6-membered non-aromatic ring system having ring carbon atoms and 1 to 3 ring heteroatoms. 5- to 6-membered heterocyclyl is yet alternative, which is a radical of 5- to 6-membered non-aromatic ring system having ring carbon atoms and 1 to 3 ring heteroatoms. 3- to 5-membered heterocyclyl is yet alternative, which is a radical of 3- to 5-membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the cycloalkyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocycly groups containing one heteroatom include, but are not limited to, azepanyl, oxepanyl and thiepanyl. Exemplary 5-membered heterocyclyl groups fused with a C₆ aryl (also referred as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred as 6,6-bicyclic heterocyclyl herein) include, but are not limited to, tetrahydroquinolinyl, tetrahydroisoquinolinyl, tetrahydrobenzopyranyl, tetrahydropyranopyridinyl, etc. The heterocyclyl can be substituted with one or more substituents, for example, with 1 to 5 substituents, 1 to 3 substituents or 1 substituent.

“C₆₋₁₀ aryl” refers to a radical of monocyclic or polycyclic (e.g., bicyclic) 4n+2 aromatic ring system having 6-10 ring carbon atoms and zero heteroatoms (e.g., having 6 or 10 shared π electrons in a cyclic array). In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“5- to 14-membered heteroaryl” refers to a radical of 5- to 14-membered monocyclic or bicyclic 4n+2 aromatic ring system (e.g., having 6, 10 or 14 shared π electrons in a cyclic array) having ring carbon atoms and 1-4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 10-membered heteroaryl groups are alternative, which are radicals of 5- to 10-membered monocyclic or bicyclic 4n+2 aromatic ring systems having ring carbon atoms and 1-4 ring heteroatoms. In some embodiments, 6- to 10-membered heteroaryl groups are alternative, which are radicals of 6- to 10-membered monocyclic or bicyclic 4n+2 aromatic ring systems having ring carbon atoms and 1-4 ring heteroatoms. In some embodiments, 5- to 9-membered heteroaryl groups are alternative, which are radicals of 5- to 9-membered monocyclic or bicyclic 4n+2 aromatic ring systems having ring carbon atoms and 1-4 ring heteroatoms. In other embodiments, 5- to 6-membered heteroaryl groups are yet alternative, which are radicals of 5- to 6-membered monocyclic or bicyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazoly), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“Cycloalkylene”, “heterocyclylene”, “arylene” or “heteroarylene” is a divalent group formed by removing another hydrogen from “cycloalkyl”, “heterocyclyl”, “aryl” or “heteroaryl” as defined above, and may be substituted or unsubstituted. For example, “C₅₋₇ cycloalkylene” refers to a divalent group formed by removing another hydrogen from C₅₋₇ cycloalkyl, “5- to 8-membered heterocyclylene” refers to a divalent group formed by removing another hydrogen from 5- to 8-membered heterocyclyl, “C₆₋₁₀ arylene” refers to a divalent group formed by removing another hydrogen from C₆₋₁₀ aryl, and “5-to 6-membered heteroarylene” refers to a divalent group formed by removing another hydrogen from 5- to 6-membered heteroaryl.

Alkyl, alkenyl, alkynyl, cycloalkyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa} (OR^{cc}), alkyl, haloalkyl, alkenyl, alkynyl, cycloalkyl, heterocyclyl, aryl and heteroaryl, wherein each of the alkyl, alkenyl, alkynyl, cycloalkyl, heterocyclyl, aryl and heteroaryl is independently substituted with 0, 1, 2, 3, 4 or 5 R^{dd} groups;
or two geminal hydrogen on a carbon atom are replaced with ═O, ═S, ═NN(R^{bb})₂, ═NR^{bb}C(═O)R^{aa}, ═NNR^{bb}C(═O)OR^{aa}, ═NNR^{bb}S(═O)₂R^{aa}, ═NR^{bb} or =NOR^{cc} groups;
each of the R^{aa} is independently selected from alkyl, haloalkyl, alkenyl, alkynyl, cycloalkyl, heterocyclyl, aryl and heteroaryl, or two of the R^{aa} groups are combined to form a heterocyclyl or heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{bb} is independently selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{cc} is independently selected from hydrogen, alkyl, haloalkyl, alkenyl, alkynyl, cycloalkyl, heterocyclyl, aryl and heteroaryl, or two R^{cc} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{dd} is independently selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents can be combined to form ═O or ═S;
each of the R^{ee} is independently selected from alkyl, haloalkyl, alkenyl, alkynyl, cycloalkyl, aryl, heterocyclyl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups;
each of the R^{ff} is independently selected from hydrogen, alkyl, haloalkyl, alkenyl, alkynyl, cycloalkyl, heterocyclyl, aryl and heteroaryl, or two R^{f} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{gg} groups;
each of the R^{gg} is independently selected from halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆alkyl)(C₁₋₆ alkyl), —N(OH)(C₁₋₆alkyl), —NH(OH), —SH, —SC₁₋₆alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆alkyl)₃, —C(═S)N(C₁₋₆alkyl)₂, C(═S)NH(C₁₋₆alkyl), C(═S)NH₂, —C(═O)S(C₁₋₆alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆alkyl)₂, C₁₋₆alkyl, C₁₋₆ haloalkyl, C₂-C₆ alkenyl, C₂-C₆ alkynyl, C₃-C₇ cycloalkyl, C₆-C₁₀ aryl, C₃-C₇ heterocyclyl, and C₅-C₁₀ heteroaryl; or two geminal R^{gg} substituents may combine to form ═O or ═S; wherein X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups attached to a nitrogen atom combine to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definitions

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “humam”, “patient” and “subject” can be used interchangeably herein.

“Disease,” “disorder,” and “condition” can be used interchangeably herein.

Generally, the “effective amount” of a compound refers to an amount sufficient to elicit a target biological response. As understood by those skilled in the art, the effective amount of the compound of the disclosure can vary depending on the following factors, such as the desired biological endpoint, the pharmacokinetics of the compound, the diseases being treated, the mode of administration, and the age, health status and symptoms of the subjects. The effective amount includes therapeutically effective amount and prophylactically effective amount.

“Combination” and related terms refer to the simultaneous or sequential administration of the compounds of the present disclosure and other therapeutic agents. For example, the compounds of the present disclosure can be administered simultaneously or sequentially in separate unit dosage with other therapeutic agents, or simultaneously in a single unit dosage with other therapeutic agents.

### BRIEF DESCRIPTION OF THE DRAWINGS

FIG. 1 shows the inhibitory IC₅₀(nM) of compound A1 on SW48 cells.

FIG. 2 shows the inhibitory IC₅₀(nM) of compound H1 on SW48 cells.

FIG. 3 shows the inhibitory IC₅₀(nM) of compound H13 on SW48 cells.

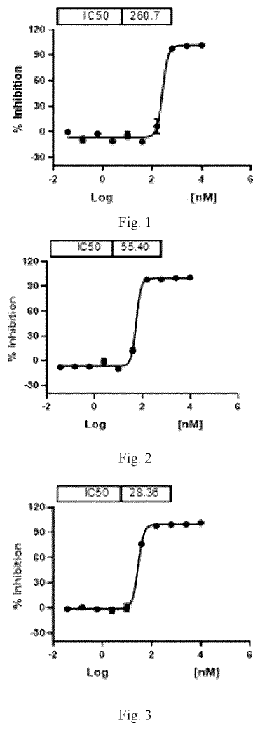

### DETAILED DESCRIPTION OF THE INVENTION

“Compound(s) of the present disclosure” herein refers to the following compounds of formula (I) (including sub-formulae, such as formula (II), (II-1), (II-2), (II-3), (II-4), (II-5), (III), (III-1), (III-2), (IV) or (V)), a pharmaceutically acceptable salt, enantiomer, diastereoisomer or isotopic variant thereof, and mixtures thereof.

In one embodiment, the present disclosure provides a compound of formula (I), or a pharmaceutically acceptable salt, isotopic variant, tautomer, or stereoisomer thereof:

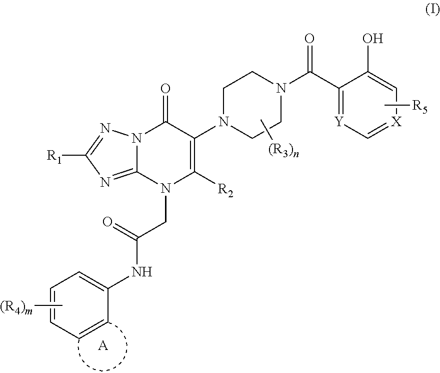

wherein,
[symbol] represents a single bond or a double bond;
X and Y are each independently selected from CH and N, and at least one of X and Y is N;
R₁ is selected from

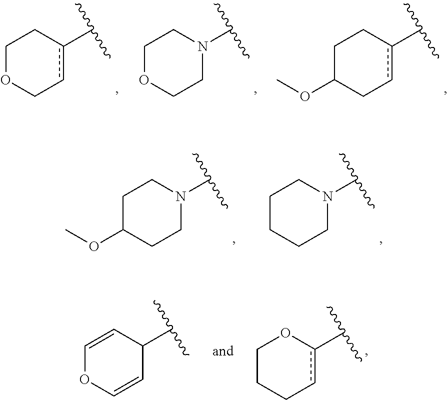

alternatively, R₁ is selected from

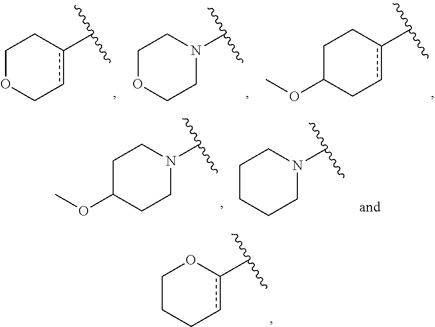

and the R₁ is optionally substituted with 1 or 2 Rₓ;
R₂ is selected from H, C₁₋₆ alkyl, C₁₋₆ haloalkyl, C₁₋₆ alkoxy, C₁₋₆ alkylthio and C₃₋₆ cycloalkyl;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

[symbol]

In one embodiment, [symbol] represents a single bond; in another embodiment, [symbol] represents a double bond.

### X and Y

In one embodiment, X is CH; in another embodiment, X is N.

In one embodiment, Y is CH; in another embodiment, Y is N.

In one embodiment, at least one of X and Y is N.

### Ring A

In one embodiment, ring A forms a fused ring with the benzene ring to which it is attached.

In one embodiment, ring A is a 5- to 6-membered heteroaryl; in another embodiment, ring A is a 5- to 7-membered heterocyclyl; in another embodiment, ring A is a 5- to 6-membered heteroaryl, said 5- to 6-membered heteroaryl containing 1 heteroatom selected from O, S and N; in another embodiment, ring A is a 5-membered heteroaryl, said 5-membered heteroaryl containing 1 heteroatom selected from O, S and N.

In a specific embodiment, ring A is

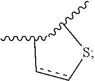

in another specific embodiment, ring A is

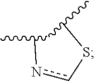

in another specific embodiment, ring A is

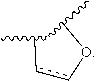

In a more specific embodiment, ring A is

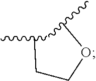

in another specific embodiment, ring A is

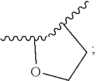

in another specific embodiment, ring A is

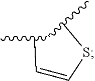

in another specific embodiment, ring A is

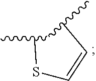

in another specific embodiment, ring A is

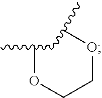

in another specific embodiment, ring A is

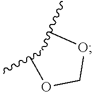

in another specific embodiment, ring A is

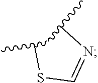

in another specific embodiment, ring A is

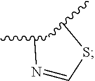

in another specific embodiment, ring A is

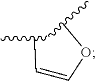

in another specific embodiment, ring A is

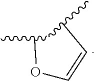

### R₁

In one embodiment, R₁ is

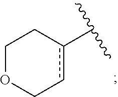

in another specific embodiment, R₁ is

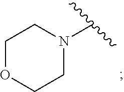 [SMILES: *N1CCOCC1]

in another specific embodiment, R₁ is

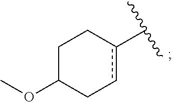

in another specific embodiment, R₁ is

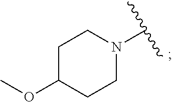 [SMILES: *N1CCC(OC)CC1]

in another specific embodiment, R₁ is

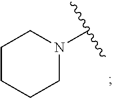 [SMILES: *N1CCCCC1 ; ";"]

in another specific embodiment, R₁ is

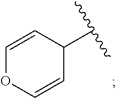 [SMILES: *C1C=COC=C1 ; ";"]

in another specific embodiment, R₁ is

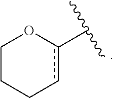

In one embodiment, the R₁ is unsubstituted; in one embodiment, the R₁ is substituted with 1 Rₓ; in one embodiment, the R₁ is substituted with 2 Rₓ.

### R₂

In one embodiment, R₂ is H; in another embodiment, R₂ is C₁₋₆ alkyl; in another embodiment, R₂ is C₁₋₄ alkyl, alternatively CH₂CH₃; In one embodiment, R₂ is C₁₋₆ haloalkyl; In one embodiment, R₂ is C₁₋₆ alkoxy; In one embodiment, R₂ is C₁₋₆ alkylthio; In one embodiment, R₂ is C₃₋₆ cycloalkyl.

### R₃

In one embodiment, R₃ is H; in another embodiment, R₃ is halogen; in another embodiment, R₃ is CN; in another embodiment, R₃ is C₁₋₆ alkyl; in another embodiment, R₃ is C₁₋₄ alkyl, such as H or CH₃; in another embodiment, R₃ is C₁₋₆ haloalkyl; in another embodiment, R₃ is C₃₋₆ cycloalkyl; in another embodiment, two R₃ on the same or different carbon atoms are taken together with the carbon atoms to which they are attached to form a 3- to 6-membered spiro or bridged ring.

In a specific embodiment, R₃ is H; in another specific embodiment, R₃ is F; in another embodiment, R₃ is CN; in another specific embodiment, R₃ is methyl; in another specific embodiment, R₃ is ethyl; in another specific embodiment, R₃ is trifluoromethyl; in another specific embodiment, R₃ is cyclopropyl; in another specific embodiment, two R₃ on the same carbon atom are taken together with the carbon atom to which they are attached to form cyclopropyl or cyclobutyl.

### R₄

In one embodiment, R₄ is H; in another embodiment, R₄ is halogen; in another embodiment, R₄ is CN; in another embodiment, R₄ is SFs; in another embodiment, R₄ is —SCF₃; in another embodiment, R₄ is C₁₋₆ alkyl; in another embodiment, R₄ is C₁₋₆ haloalkyl; in another embodiment, R₄ is C₁₋₆ alkylthio; in another embodiment, R₄ is C₃₋₆ cycloalkyl; in another embodiment, R₄ is 4- to 10-membered heterocyclyl.

In a specific embodiment, R₄ is H; in another specific embodiment, R₄ is F; in another specific embodiment, R₄ is Cl; in another specific embodiment, R₄ is Br; in another specific embodiment, R₄ is CN; in another specific embodiment, R₄ is SFs; in another specific embodiment, R₄ is methyl; in another specific embodiment, R₄ is ethyl; in another specific embodiment, R₄ is trifluoromethyl; in another specific embodiment, R₄ is difluoromethyl; in another specific embodiment, R₄ is methylthio; in another specific embodiment, R₄ is cyclopropyl.

### R₅

In one embodiment, R₅ is H; in another embodiment, R₅ is halogen; in another embodiment, R₅ is C₁₋₆ alkyl; in another embodiment, R₅ is C_{1-.6} haloalkyl; in another embodiment, R₅ is C₁₋₆ alkoxy; in another embodiment, R₅ is C₁₋₆ alkylthio; in another embodiment, R₅ is C₃₋₆ cycloalkyl.

In a specific embodiment, R₅ is H; in another specific embodiment, R₅ is F; in another specific embodiment, R₅ is C₁; in another specific embodiment, R₅ is Br; in another specific embodiment, R₅ is methyl; in another specific embodiment, R₅ is ethyl; in another specific embodiment, R₅ is trifluoromethyl; in another specific embodiment, R₅ is methoxy; in another specific embodiment, R₅ is methylthio; in another specific embodiment, R₅ is cyclopropyl.

### Rₓ

In one embodiment, Rₓ is H; in another embodiment, Rₓ is C₁₋₆ alkyl; in another embodiment, Rₓ is C₁₋₆ haloalkyl; in another embodiment, Rₓ is C₁₋₆ alkoxy; in another embodiment, Rₓ is CN; in another embodiment, Rₓ is NH₂; in another embodiment, Rₓ is —C(O)Rₐ; in another embodiment, Rₓ is —C(O)ORₐ; in another embodiment, Rₓ is —(CH₂)ₚ—ORₐ; in another embodiment, Rₓ is —P(O)—(Rₐ)₂; in another embodiment, Rₓ is —S(O)₂—Rₐ.

In a specific embodiment, Rₓ is H; in another specific embodiment, Rₓ is methyl; in another specific embodiment, Rₓ is trifluoromethyl; in another specific embodiment, Rₓ is methoxy; in another specific embodiment, Rₓ is CN; in another specific embodiment, Rₓ is NH₂.

### Rₐ

In one embodiment, Rₐ is H; in another embodiment, Rₐ is C₁₋₆ alkyl; in another embodiment, Rₐ is C₃₋₆ cycloalkyl.

m

In one embodiment, m is selected from 0, 1, 2 and 3.

n

In one embodiment, n is selected from 0, 1 and 2.

p

In one embodiment, p is selected from 0, 1 and 2.

Any technical solution in any one of the above specific embodiments, or any combination thereof, may be combined with any technical solution in other specific embodiments or any combination thereof. For example, any technical solution of ring A or any combination thereof may be combined with any technical solution of X, Y, R₁, R₂, R₃, R₄, R₅, Rₓ, Rₐ, m, n and p, etc., or any combination thereof. The present disclosure is intended to include all combinations of such technical solutions, which are not exhaustively listed here to save space.

In another embodiment, the present disclosure provides a compound of formula (II), or a pharmaceutically acceptable salt, isotopic variant, tautomer, or stereoisomer thereof:

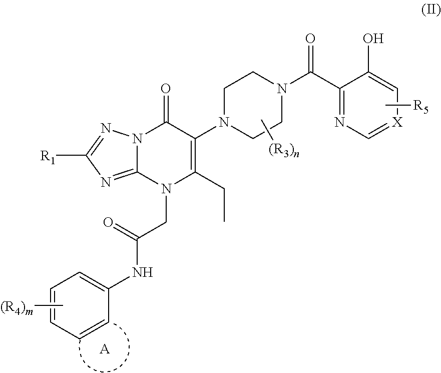

wherein
[symbol] represents a single bond or a double bond;
X is selected from CH and N;
R₁ is selected from

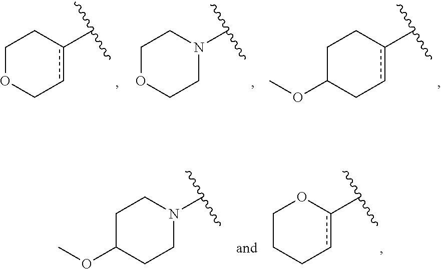

and the R₁ is optionally substituted with 1 or 2Rₓ;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

In another embodiment, the present disclosure provides a compound of the following formula (II-1), formula (II-2), formula (II-3), formula (II-4), formula (II-5), formula (II-6) or formula (II-7), or a pharmaceutically acceptable salt, isotopic variant, tautomer, or stereoisomer thereof:

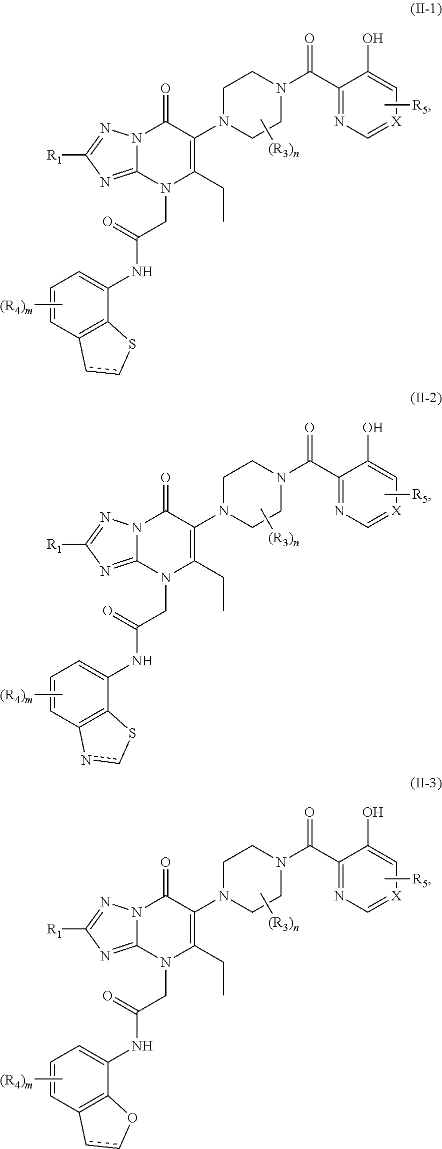

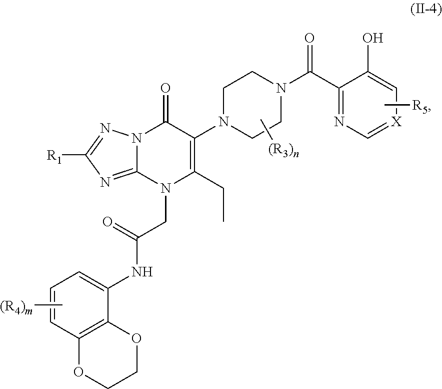

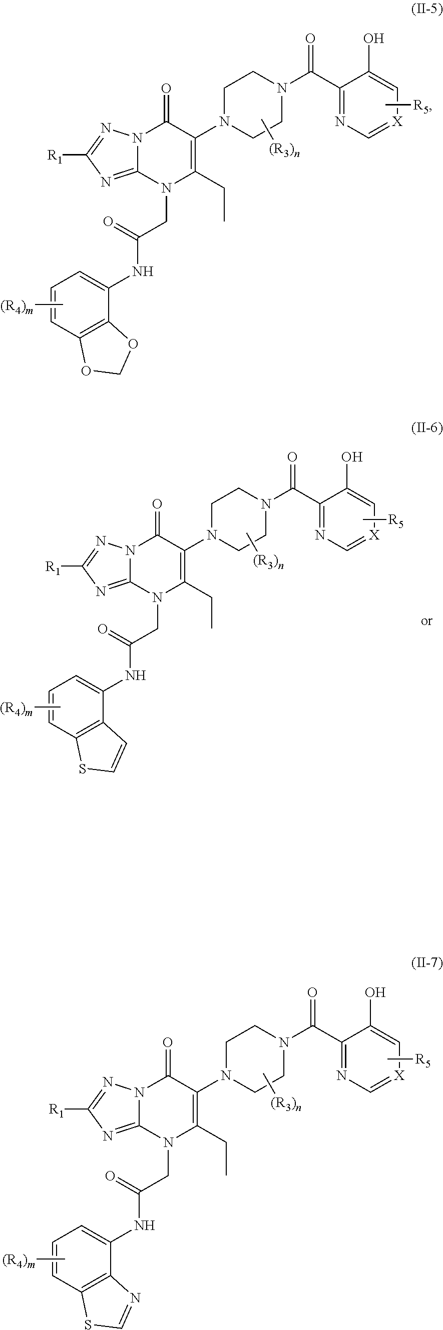

wherein
[symbol] represents a single bond or a double bond;
X is selected from CH and N;
R₁ is selected from

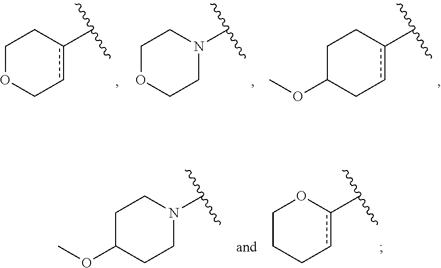

the R₁ is optionally substituted with 1 or 2 Rₓ;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

In another more specific embodiment, the present disclosure provides a compound of the above formula (II-1), formula (II-2), formula (II-3), formula (II-4), formula (II-5), formula (II-6) or formula (II-7), or a pharmaceutically acceptable salt, isotopic variant, tautomer, or stereoisomer thereof, wherein:
[symbol] represents a single bond or a double bond;
X is selected from CH and N;
R₁ is selected from

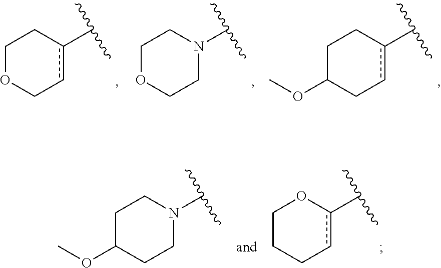

The R₁ is optionally substituted with 1 or 2 Rₓ;
R₃ is independently selected from H, F, CN, methyl, ethyl, trifluoromethyl and cyclopropyl; or two R₃ are taken together with the carbon atom(s) to which they are attached to form cyclopropyl or cyclobutyl;
R₄ is independently selected from H, F, Cl, Br, CN, SF₅, methyl, ethyl, trifluoromethyl, difluoromethyl, methylthio and cyclopropyl;
R₅ is selected from H, F, Cl, Br, methyl, ethyl, trifluoromethyl, methoxy, methylthio and cyclopropyl;
Rₓ is selected from H, methyl, trifluoromethyl, methoxy, CN and NH₂;
m is selected from 0, 1, 2 and 3;
n is selected from 0, 1 and 2;
p is selected from 0, 1 and 2.

In another embodiment, the present disclosure provides a compound of formula (III), or a pharmaceutically acceptable salt, isotopic variant, tautomer, or stereoisomer thereof:

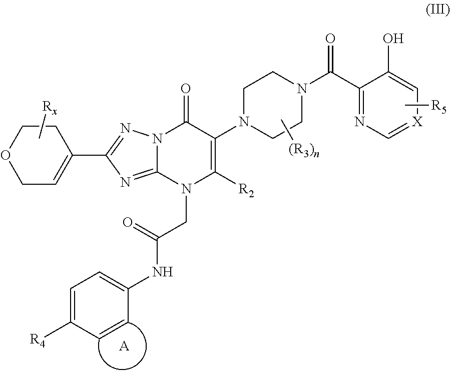

wherein,
X is selected from CH and N;
Ring A is a 5- to 6-membered heteroaryl, wherein the 5- to 6-membered heteroaryl group contains 1 heteroatom selected from O, S and N;
Rₓ is selected from H, C₁₋₆ alkyl, C₁₋₆ haloalkyl and C₁₋₆ alkoxy;
R₂ is selected from H, C₁₋₆ alkyl, C₁₋₆ haloalkyl and C₁₋₆ alkoxy;
R₃ is selected from H, halogen, C₁₋₆ alkyl and C₁₋₆ haloalkyl;
R₄ is selected from H, halogen, C₁₋₆ alkyl and C₁₋₆ haloalkyl;
R₅ is selected from H, halogen, C₁₋₆ alkyl, C₁₋₆ haloalkyl and C₁₋₆ alkoxy;
n is 0, 1 or 2.

In another specific embodiment, the present disclosure provides a compound of formula (III) as described above, or a pharmaceutically acceptable salt, isotopic variant, tautomer, or stereoisomer thereof, wherein,
X is selected from CH and N;
Ring A is a 5- to 6-membered heteroaryl, wherein the 5- to 6-membered heteroaryl group contains 1 heteroatom selected from O, S and N;
Rₓ is selected from H, C₁₋₆ alkyl and C₁₋₆ haloalkyl;
R₂ is selected from H, C₁₋₆ alkyl and C₁₋₆ haloalkyl;
R₃ is selected from H and C₁₋₆ alkyl;
R₄ is selected from halogen and C₁₋₆ haloalkyl;
R₅ is selected from H, halogen, C₁₋₆ alkyl and C₁₋₆ haloalkyl;
n is 0, 1 or 2.

In another specific embodiment, the present disclosure provides a compound of formula (III) as described above, or a pharmaceutically acceptable salt, isotopic variant, tautomer, or stereoisomer thereof, wherein,
X is selected from CH and N;
Ring A is a 5-membered heteroaryl group, wherein the 5-membered heteroaryl contains 1 heteroatom selected from O, S and N;
Rₓ is selected from H and C₁₋₄ alkyl, alternatively H;
R₂ is selected from H and C₁₋₄ alkyl, alternatively CH₂CH₃;
R₃ is selected from H and C₁₋₄ alkyl, such as H and CH₃;
R₄ is selected from halogen and C₁₋₄ haloalkyl, such as Br, Cl and CF₃;
R₅ is selected from H, halogen and C₁₋₄ alkyl, alternatively F, Cl and CH₃;
n is 0, 1 or 2.

In another specific embodiment, the present disclosure provides a compound of formula (III-1) or formula (III-2), or a pharmaceutically acceptable salt, isotopic variant, tautomer, or stereoisomer thereof:

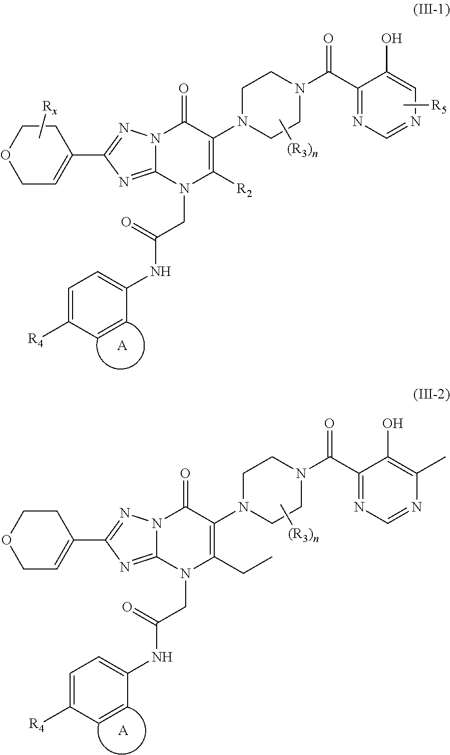

wherein,
the variables are as defined herein.

In another specific embodiment, the present disclosure provides a compound of formula (IV) or formula (V), or a pharmaceutically acceptable salt, isotopic variant, tautomer, or stereoisomer thereof:

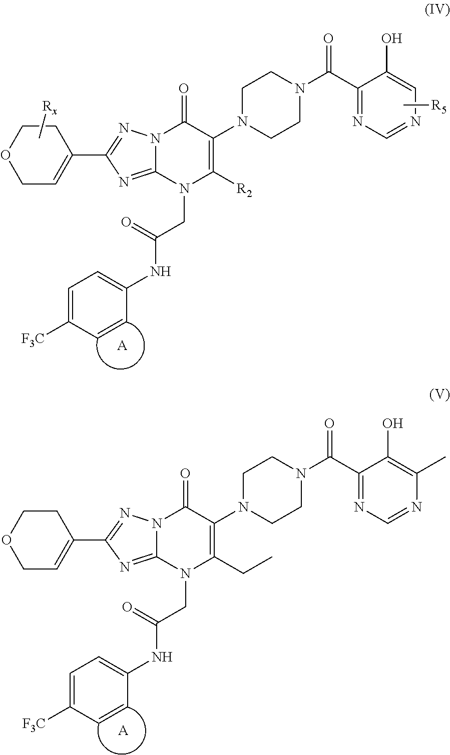

wherein,
Ring A is a 5- to 6-membered heteroaryl, wherein the 5- to 6-membered heteroaryl contains 1 heteroatom selected from O, S and N;
Rₓ is selected from H, C₁₋₆ alkyl, C₁₋₆ haloalkyl and C₁₋₆ alkoxy;
R₂ is selected from H, C₁₋₆ alkyl, C₁₋₆ haloalkyl and C₁₋₆ alkoxy;
R₅ is selected from H, halogen, C₁₋₆ alkyl, C₁₋₆ haloalkyl and C₁₋₆ alkoxy.

In another specific embodiment, the present disclosure provides a compound of the above formula (IV) or formula (V), or a pharmaceutically acceptable salt, isotopic variant, tautomer, or stereoisomer thereof, wherein,
Ring A is a 5-membered heteroaryl group, wherein the 5-membered heteroaryl contains 1 heteroatom selected from O, S and N;
Rₓ is selected from H, C₁₋₆ alkyl and C₁₋₆ haloalkyl;
R₂ is selected from H, C₁₋₆ alkyl and C₁₋₆ haloalkyl;
R₅ is selected from H, halogen, C₁₋₆ alkyl and C₁₋₆ haloalkyl.

In another specific embodiment, the present disclosure provides a compound of the above formula (IV) or formula (V), or a pharmaceutically acceptable salt, isotopic variant, tautomer, or stereoisomer thereof, wherein,
Ring A is a 5-membered heteroaryl group, wherein the 5-membered heteroaryl contains 1 heteroatom selected from O, S and N;
Rₓ is selected from H and C₁₋₄ alkyl, alternatively H;
R₂ is selected from H and C₁₋₄ alkyl, alternatively CH₂CH₃;
R₅ is selected from H and C₁₋₄ alkyl, alternatively CH₃.

In another more specific embodiment, the compound is selected from the group consisting of:

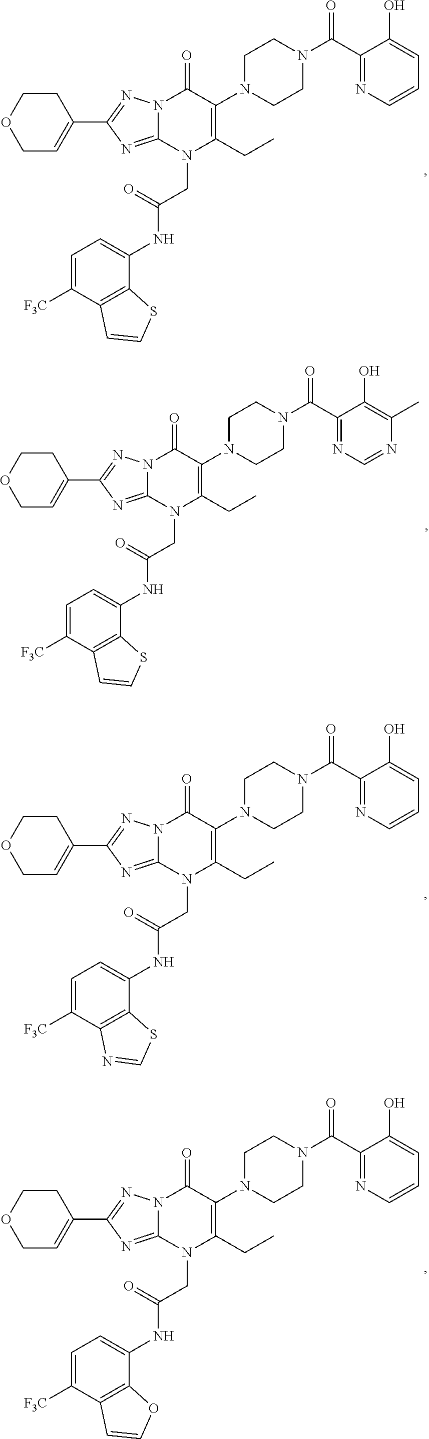 [SMILES: CCc1c(N2CCN(C(=O)c3ncccc3O)CC2)c(=O)n2nc(C3=CCOCC3)nc2n1CC(=O)Nc1ccc(C(F)(F)F)c2ccsc12 ; CCc1c(N2CCN(C(=O)c3ncnc(C)c3O)CC2)c(=O)n2nc(C3=CCOCC3)nc2n1CC(=O)Nc1ccc(C(F)(F)F)c2ccsc12 ; CCc1c(N2CCN(C(=O)c3ncccc3O)CC2)c(=O)n2nc(C3=CCOCC3)nc2n1CC(=O)Nc1ccc(C(F)(F)F)c2ncsc12 ; CCc1c(N2CCN(C(=O)c3ncccc3O)CC2)c(=O)n2nc(C3=CCOCC3)nc2n1CC(=O)Nc1ccc(C(F)(F)F)c2ccoc12]

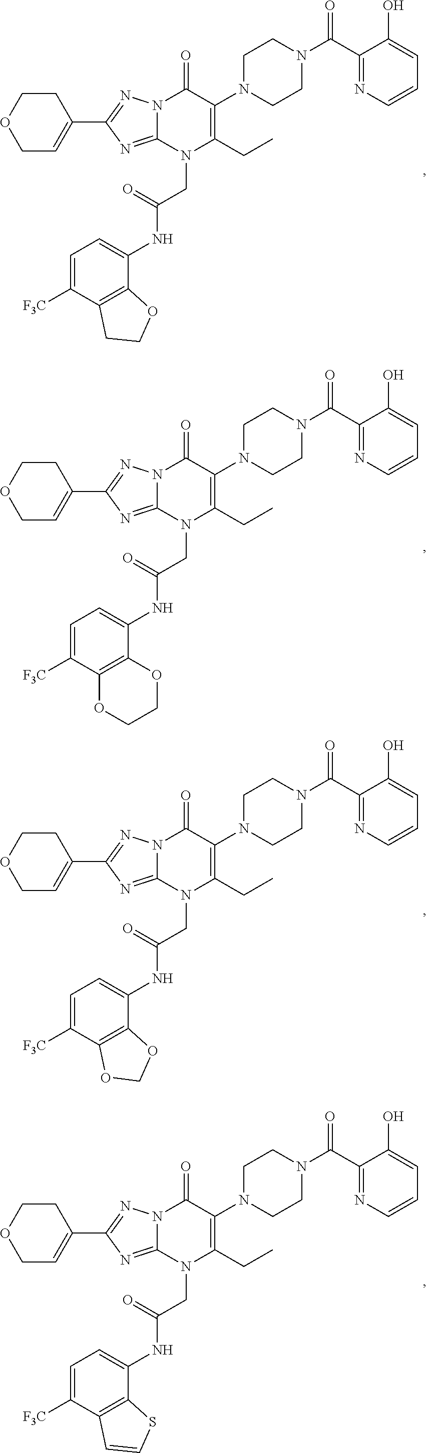 [SMILES: CCc1c(N2CCN(C(=O)c3ncccc3O)CC2)c(=O)n2nc(C3=CCOCC3)nc2n1CC(=O)Nc1ccc(C(F)(F)F)c2c1OCC2 ; CCc1c(N2CCN(C(=O)c3ncccc3O)CC2)c(=O)n2nc(C3=CCOCC3)nc2n1CC(=O)Nc1ccc(C(F)(F)F)c2c1OCCO2 ; CCc1c(N2CCN(C(=O)c3ncccc3O)CC2)c(=O)n2nc(C3=CCOCC3)nc2n1CC(=O)Nc1ccc(C(F)(F)F)c2c1OCO2 ; CCc1c(N2CCN(C(=O)c3ncccc3O)CC2)c(=O)n2nc(C3=CCOCC3)nc2n1CC(=O)Nc1ccc(C(F)(F)F)c2ccsc12]

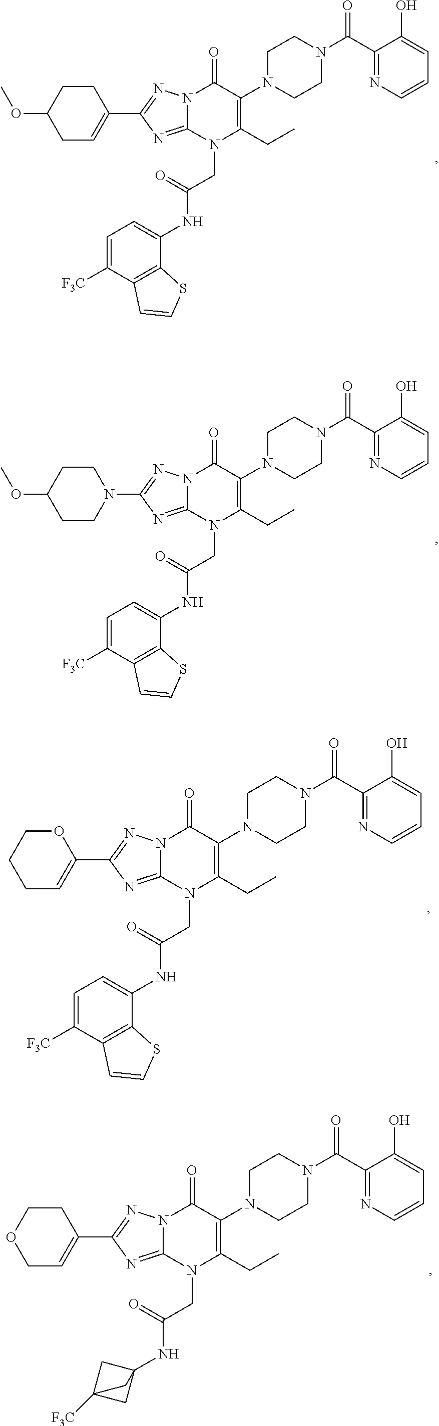 [SMILES: CCc1c(N2CCN(C(=O)c3ncccc3O)CC2)c(=O)n2nc(C3=CCC(OC)CC3)nc2n1CC(=O)Nc1ccc(C(F)(F)F)c2ccsc12 ; CCc1c(N2CCN(C(=O)c3ncccc3O)CC2)c(=O)n2nc(N3CCC(OC)CC3)nc2n1CC(=O)Nc1ccc(C(F)(F)F)c2ccsc12 ; CCc1c(N2CCN(C(=O)c3ncccc3O)CC2)c(=O)n2nc(C3=CCCCO3)nc2n1CC(=O)Nc1ccc(C(F)(F)F)c2ccsc12 ; CCc1c(N2CCN(C(=O)c3ncccc3O)CC2)c(=O)n2nc(C3=CCOCC3)nc2n1CC(=O)NC12CC(C(F)(F)F)(C1)C2]

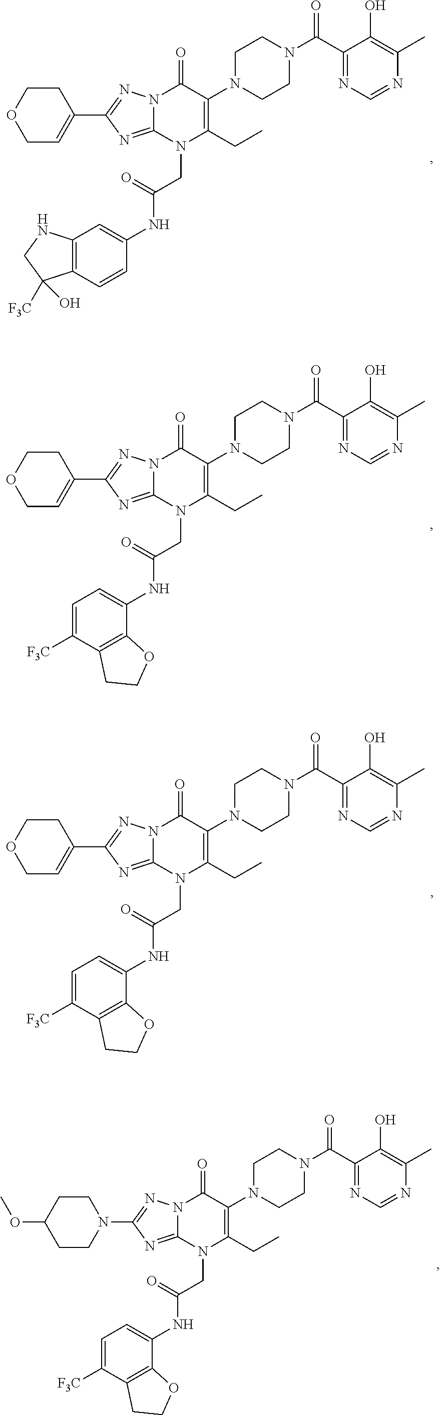

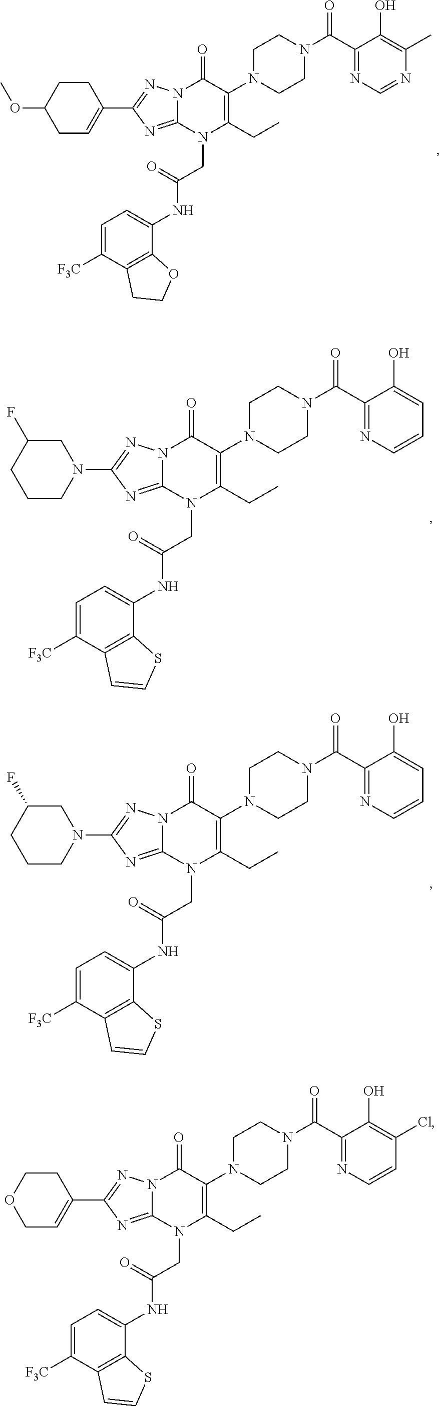

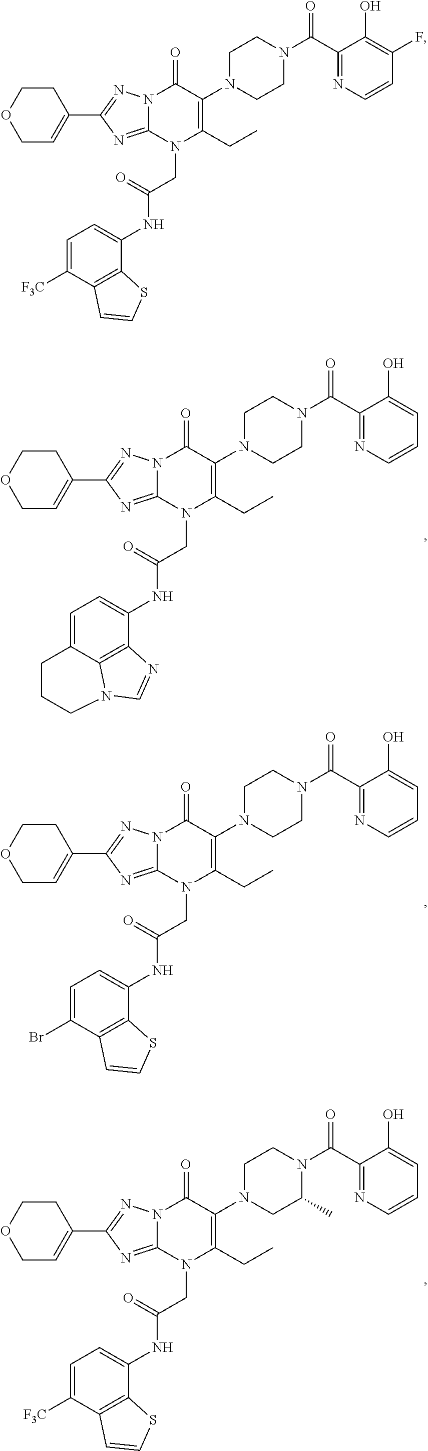 [SMILES: CCc1c(N2CCN(C(=O)c3nccc(F)c3O)CC2)c(=O)n2nc(C3=CCOCC3)nc2n1CC(=O)Nc1ccc(C(F)(F)F)c2ccsc12 ; CCc1c(N2CCN(C(=O)c3ncccc3O)CC2)c(=O)n2nc(C3=CCOCC3)nc2n1CC(=O)Nc1ccc2c3c1ncn3CCC2 ; CCc1c(N2CCN(C(=O)c3ncccc3O)CC2)c(=O)n2nc(C3=CCOCC3)nc2n1CC(=O)Nc1ccc(Br)c2ccsc12 ; CCc1c(N2CCN(C(=O)c3ncccc3O)[C@H](C)C2)c(=O)n2nc(C3=CCOCC3)nc2n1CC(=O)Nc1ccc(C(F)(F)F)c2ccsc12]

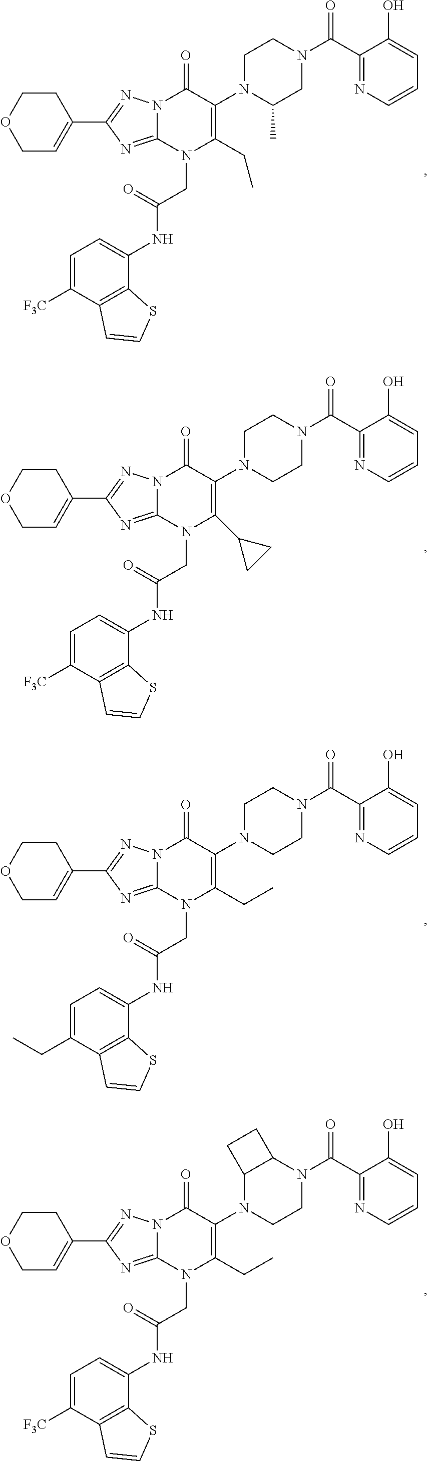 [SMILES: CCc1c(N2CCN(C(=O)c3ncccc3O)C[C@@H]2C)c(=O)n2nc(C3=CCOCC3)nc2n1CC(=O)Nc1ccc(C(F)(F)F)c2ccsc12 ; O=C(Cn1c(C2CC2)c(N2CCN(C(=O)c3ncccc3O)CC2)c(=O)n2nc(C3=CCOCC3)nc12)Nc1ccc(C(F)(F)F)c2ccsc12 ; CCc1ccc(NC(=O)Cn2c(CC)c(N3CCN(C(=O)c4ncccc4O)CC3)c(=O)n3nc(C4=CCOCC4)nc23)c2sccc12 ; CCc1c(N2CCN(C(=O)c3ncccc3O)C3CCC32)c(=O)n2nc(C3=CCOCC3)nc2n1CC(=O)Nc1ccc(C(F)(F)F)c2ccsc12]

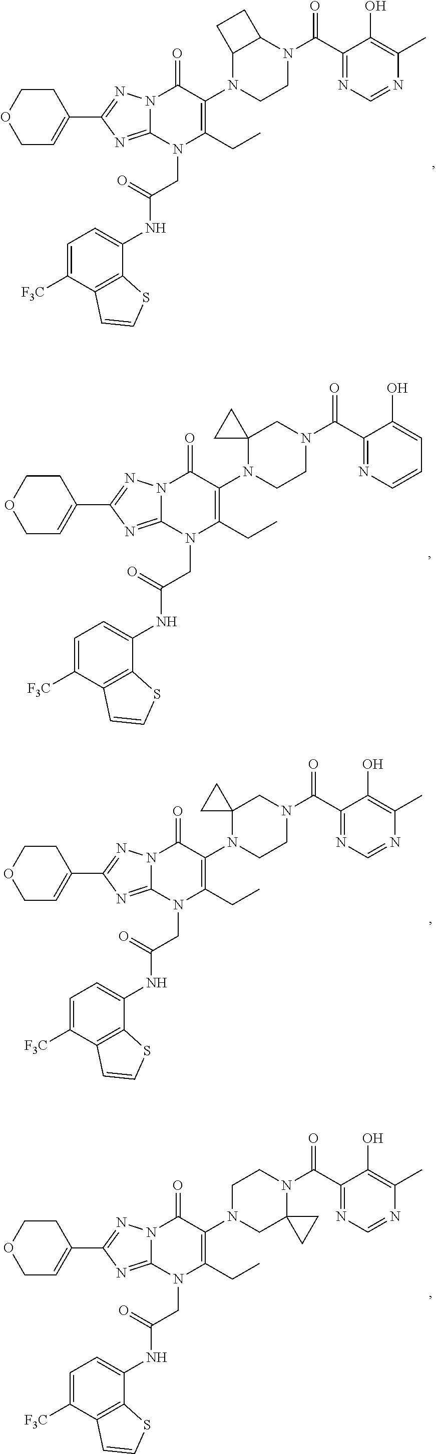 [SMILES: CCc1c(N2CCN(C(=O)c3ncnc(C)c3O)C3CCC32)c(=O)n2nc(C3=CCOCC3)nc2n1CC(=O)Nc1ccc(C(F)(F)F)c2ccsc12 ; CCc1c(N2CCN(C(=O)c3ncccc3O)CC23CC3)c(=O)n2nc(C3=CCOCC3)nc2n1CC(=O)Nc1ccc(C(F)(F)F)c2ccsc12 ; CCc1c(N2CCN(C(=O)c3ncnc(C)c3O)CC23CC3)c(=O)n2nc(C3=CCOCC3)nc2n1CC(=O)Nc1ccc(C(F)(F)F)c2ccsc12 ; CCc1c(N2CCN(C(=O)c3ncnc(C)c3O)C3(CC3)C2)c(=O)n2nc(C3=CCOCC3)nc2n1CC(=O)Nc1ccc(C(F)(F)F)c2ccsc12]

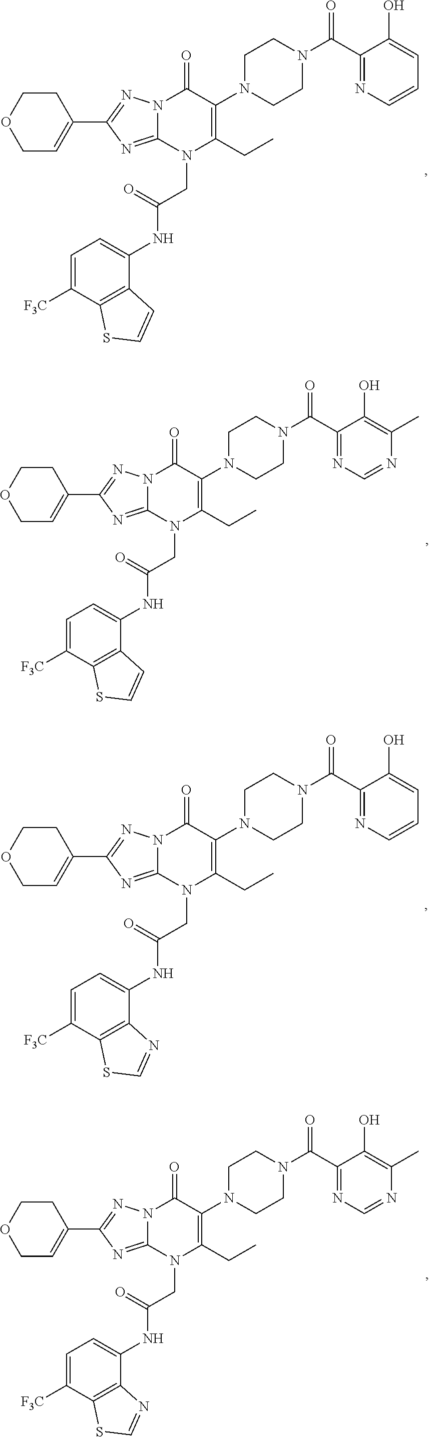 [SMILES: CCc1c(N2CCN(C(=O)c3ncccc3O)CC2)c(=O)n2nc(C3=CCOCC3)nc2n1CC(=O)Nc1ccc(C(F)(F)F)c2sccc12 ; CCc1c(N2CCN(C(=O)c3ncnc(C)c3O)CC2)c(=O)n2nc(C3=CCOCC3)nc2n1CC(=O)Nc1ccc(C(F)(F)F)c2sccc12 ; CCc1c(N2CCN(C(=O)c3ncccc3O)CC2)c(=O)n2nc(C3=CCOCC3)nc2n1CC(=O)Nc1ccc(C(F)(F)F)c2scnc12 ; CCc1c(N2CCN(C(=O)c3ncnc(C)c3O)CC2)c(=O)n2nc(C3=CCOCC3)nc2n1CC(=O)Nc1ccc(C(F)(F)F)c2scnc12]

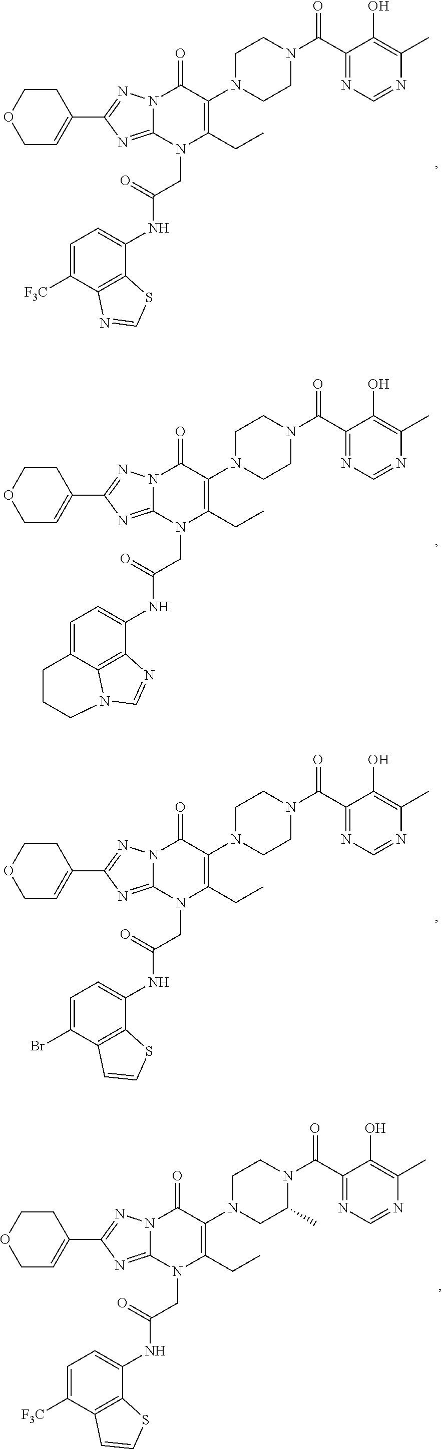 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ncsc12 ; CCc1c(N2CCN(C(=O)c3ncnc(C)c3O)CC2)c(=O)n2nc(C3=CCOCC3)nc2n1CC(=O)Nc1ccc2c3c1ncn3CCC2 ; CCc1c(N2CCN(C(=O)c3ncnc(C)c3O)CC2)c(=O)n2nc(C3=CCOCC3)nc2n1CC(=O)Nc1ccc(Br)c2ccsc12 ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)c2ccsc12]

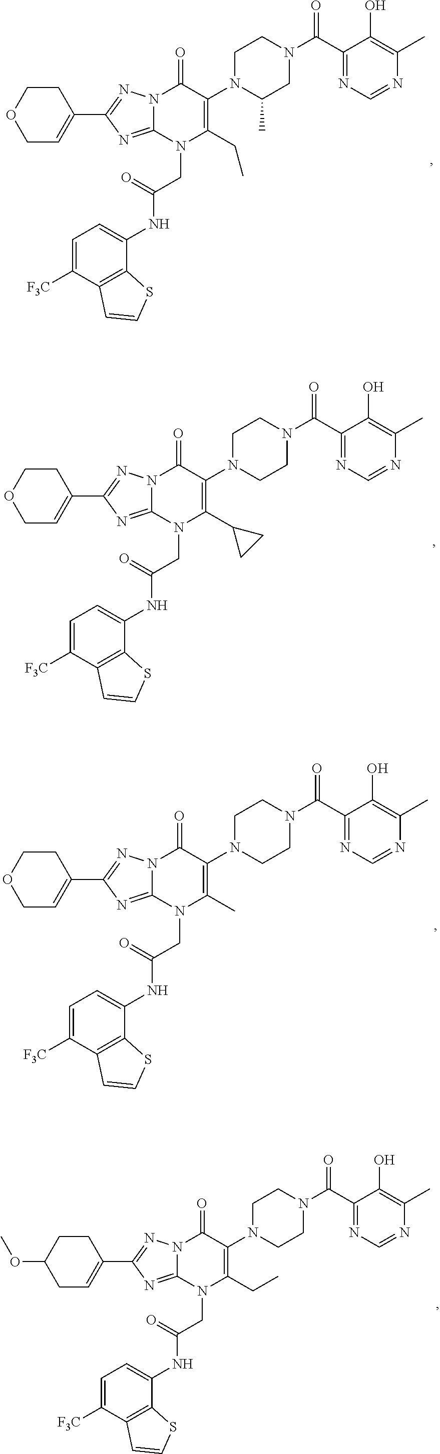

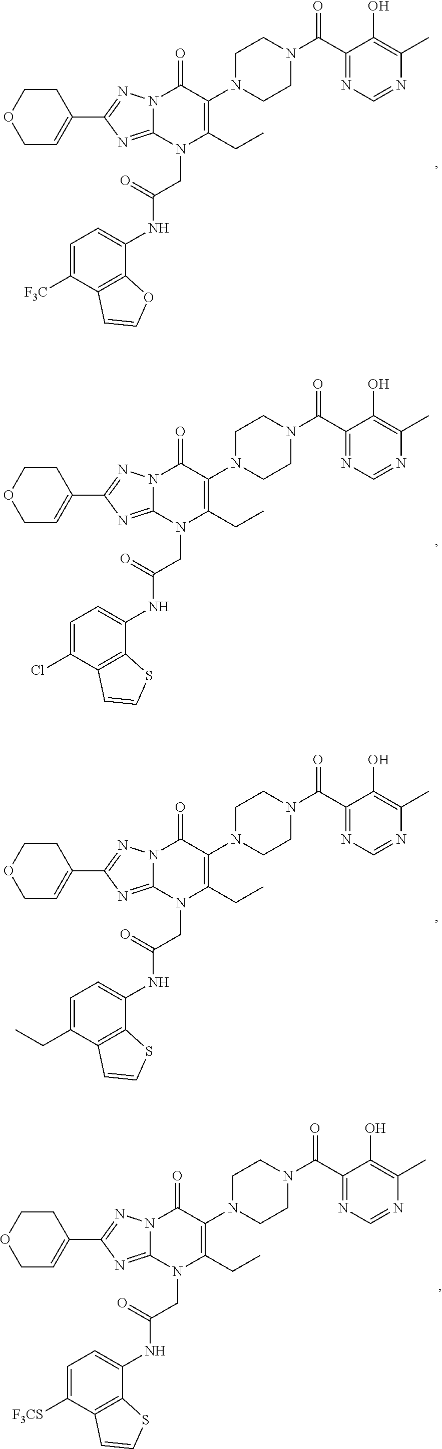

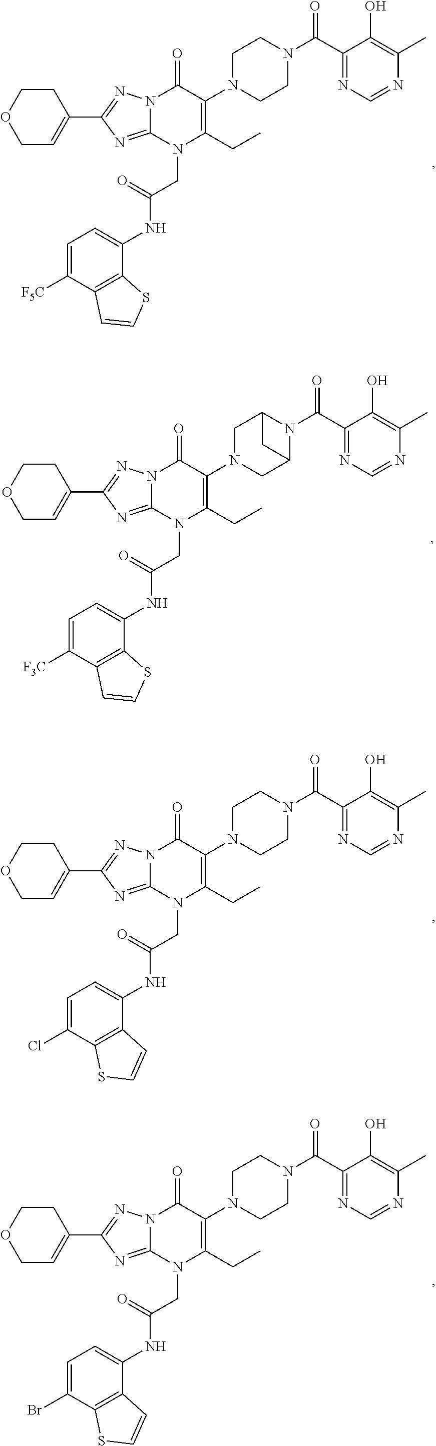

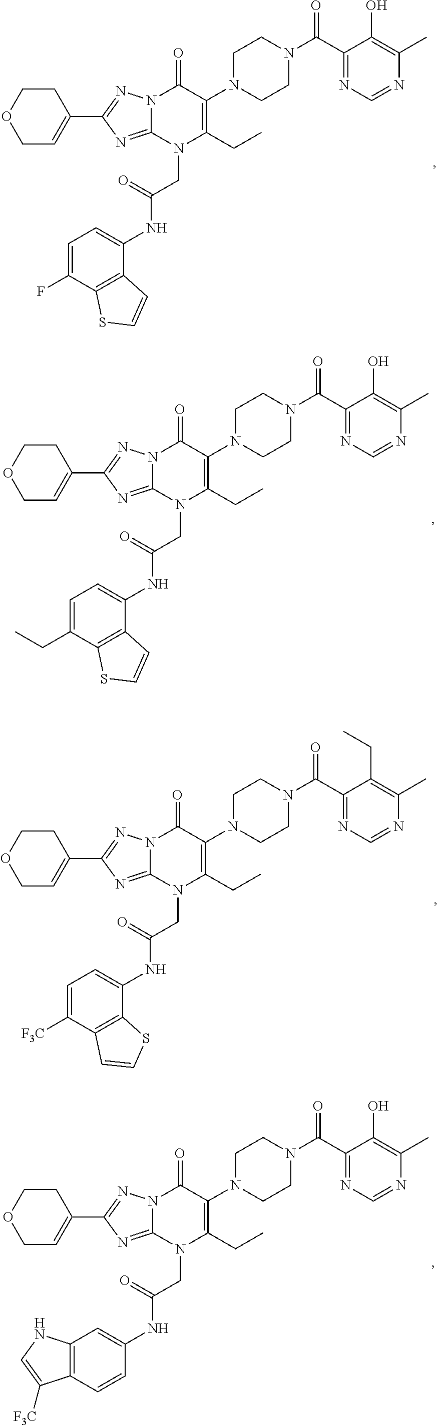 [SMILES: CCc1c(N2CCN(C(=O)c3ncnc(C)c3O)CC2)c(=O)n2nc(C3=CCOCC3)nc2n1CC(=O)Nc1ccc(F)c2sccc12 ; CCc1ccc(NC(=O)Cn2c(CC)c(N3CCN(C(=O)c4ncnc(C)c4O)CC3)c(=O)n3nc(C4=CCOCC4)nc23)c2ccsc12 ; CCc1c(C)ncnc1C(=O)N1CCN(c2c(CC)n(CC(=O)Nc3ccc(C(F)(F)F)c4ccsc34)c3nc(C4=CCOCC4)nn3c2=O)CC1 ; CCc1c(N2CCN(C(=O)c3ncnc(C)c3O)CC2)c(=O)n2nc(C3=CCOCC3)nc2n1CC(=O)Nc1ccc2c(C(F)(F)F)c[nH]c2c1]

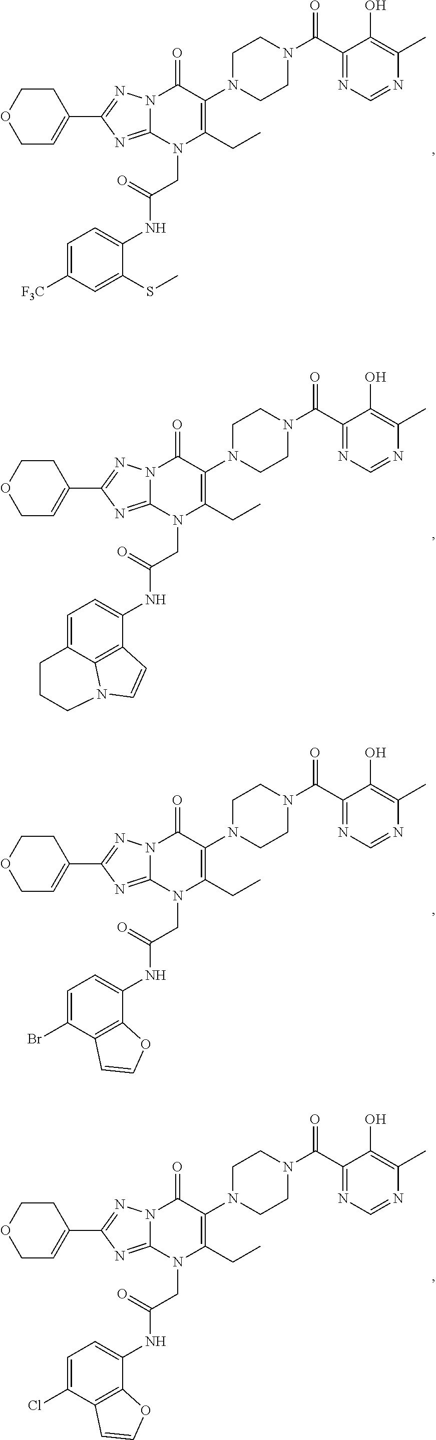 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1SC ; CCc1c(N2CCN(C(=O)c3ncnc(C)c3O)CC2)c(=O)n2nc(C3=CCOCC3)nc2n1CC(=O)Nc1ccc2c3c1ccn3CCC2 ; CCc1c(N2CCN(C(=O)c3ncnc(C)c3O)CC2)c(=O)n2nc(C3=CCOCC3)nc2n1CC(=O)Nc1ccc(Br)c2ccoc12 ; CCc1c(N2CCN(C(=O)c3ncnc(C)c3O)CC2)c(=O)n2nc(C3=CCOCC3)nc2n1CC(=O)Nc1ccc(Cl)c2ccoc12]

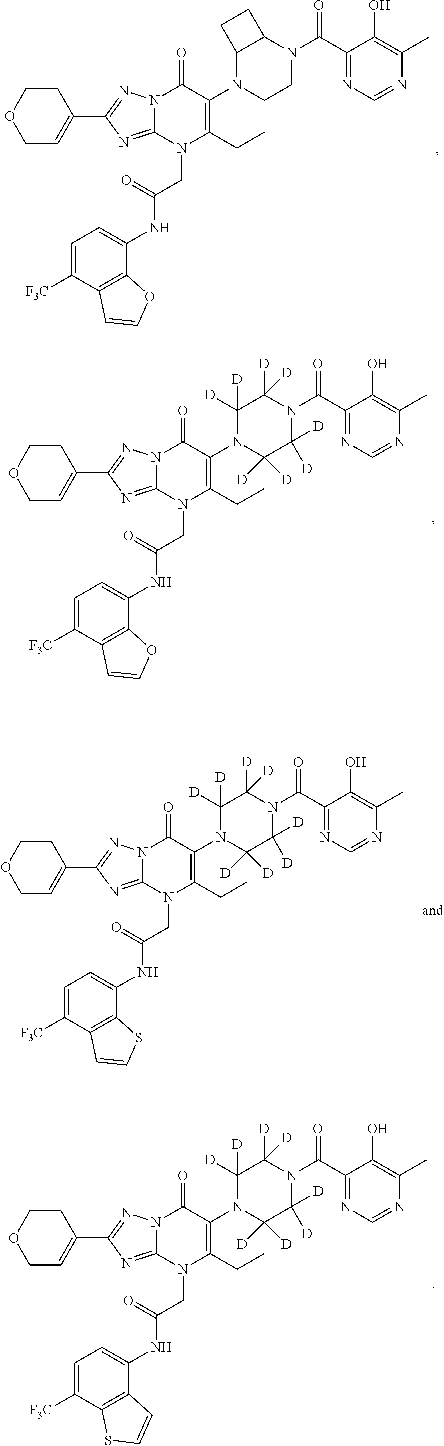

The compounds of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high-pressure liquid chromatography (HPLC) and the formation and crystallization of chiral salts; or alternative isomers can be prepared by asymmetric synthesis.

The compounds of the present disclosure may also exist as tautomers. For compounds that exist in different tautomeric forms, a compound is not limited to any specific tautomer, but is intended to encompass all tautomeric forms.

The present disclosure also comprises compounds that are labeled with isotopes (isotope variants), which are equivalent to those described in formula (A), but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as 2H, ³H, ¹³C, ¹¹C, ¹⁴C, ¹⁵N, ¹⁸O, ¹⁷O, ³¹P, ³²P, ³⁵S, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are yet alternative, because they are easy to prepare and detect. Furthermore, replaced by heavier isotopes, such as deuterium, which is ²H, may provide therapeutic benefits due to the higher metabolic stability, such as prolonging the half-life in vivo or decreasing the dosage requirements, and thus may be alternative in some cases. Isotope-labeled compounds of formula (A) of the present disclosure and prodrugs thereof can be prepared generally by using readily available isotope-labeled reagents to replace non-isotope-labeled reagents in the following schemes and/or the procedures disclosed in the examples and preparation examples.

### Pharmaceutical Compositions and Kits

In another aspect, the present disclosure provides a pharmaceutical composition comprising a compound of the present disclosure (also referred to as the “active ingredient”) and a pharmaceutically acceptable excipient. In certain embodiments, the pharmaceutical composition comprises an effective amount of the compound of the present disclosure. In certain embodiments, the pharmaceutical composition comprises a therapeutically effective amount of the compound of the present disclosure. In certain embodiments, the pharmaceutical composition comprises a prophylactically effective amount of the compound of the present disclosure.

A pharmaceutically acceptable excipient for use in the present disclosure refers to a non-toxic carrier, adjuvant or vehicle which does not destroy the pharmacological activity of the compound formulated together. Pharmaceutically acceptable carriers, adjuvants, or vehicles that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). Kits provided may include a compound disclosed herein, other therapeutic agents, and a first and a second containers (e.g., vials, ampoules, bottles, syringes, and/or dispersible packages or other materials) containing the compound disclosed herein or other therapeutic agents. In some embodiments, kits provided can also optionally include a third container containing a pharmaceutically acceptable excipient for diluting or suspending the compound disclosed herein and/or other therapeutic agent. In some embodiments, the compound disclosed herein provided in the first container and the other therapeutic agents provided in the second container is combined to form a unit dosage form.

### Administration

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compostions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or alternatively from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with alternative doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, alternatively from about 0.1 to about 20% by weight, alternatively from about 0.1 to about 10% by weight, and still alternatively from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol. See, e.g., U.S. Pat. No. 5,376,645. In certain embodiments, the formulation comprises hexapropyl-β-cyclodextrin (e.g., 10-50% in water).

### EXAMPLE

The reagents used in the present disclosure are commercial reagents purchased directly or synthesized by common methods well known in the art.

Notes on commonly used abbreviations:

PE=petroleum ether; EA=ethyl acetate; MeOH=methanol; DCM=dichloromethane; DCE=dichloroethane; CH₃CN=acetonitrile; 1,4-dioxane=1,4-dioxane; DMSO=dimethyl sulfoxide; HFIP=hexafluoroisopropanol; DMF=N,N-dimethylformamide; Hex=n-hexane; IPA=isopropanol; NMP=N-methylpyrrolidone; NMO=N-methylmorpholine-N-oxide; TEA=triethylamine; DIEA=diisopropylethylamine; CuI=cuprous iodide; CuCN=cuprous cyanide; triphosgene=triphosgene; p-TsOH=p-toluenesulfonic acid; T₃P=1-propylphosphoric acid cyclic anhydride; TsN₃=p-toluenesulfonyl azide; PPA=polyphosphoric acid; SEM-Cl=2-(trimethylsilyl)ethoxymethyl chloride; HMPA=hexamethylphosphoramide; BINAP=1,1′-binaphthyl-2,2′-bis(diphenylphosphine); TMSCF₃=trifluoromethyltrimethylsilane; Bpin=boric acid pinacol ester; B₂pin=Bis(pinacolato)diboron; NBS=N-bromosuccinimide.

MSI-H=microsatellite instability-high; MSI-L=microsatellite instability-low; MSS=microsatellite stable.

### Example 1

### Preparation of Key Intermediates

### Preparation of Intermediate a 1

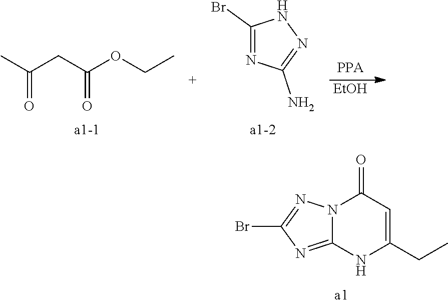

Procedure: The raw material ethyl acetoacetate a1-1 (9.0 g, 69.2 mmol) and 5-bromo-1-H-3-amino-1,2,4-triazole a1-2 (11.3 g, 69.2 mmol) were dissolved in ethanol (90 mL), and polyphosphoric acid (PPA, 8.0 g, 69.2 mmol) was slowly added. After complete addition, the mixture was heated to 80° C. to react for 12 hours. The mixture was cooled to room temperature, and evaporated under reduced pressure to remove the solvent. The reaction solution was poured into 100 mL of ice water, adjusted to pH of about 8 with saturated aqueous sodium bicarbonate solution, extracted with dichloromethane, dried over anhydrous sodium sulfate, and concentrated to obtain a1 (7.5 g) as a white solid, with a yield of 45%. LCMS ESI-MS m/z: 243 [M+H]⁺.

### Preparation of intermediates a5, a12-a19, and a31

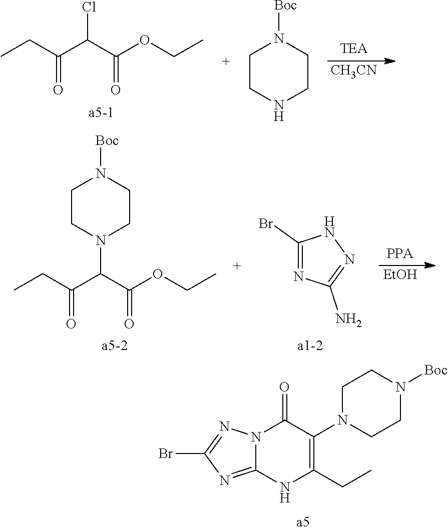

Step 1: The raw material a5-1 (24.0 g, 134 mmol) and tert-butyl piperazine-1-carboxylate (25.0 g, 134 mmol) were dissolved in 240 mL of acetonitrile, and TEA (40.8 g, 403 mmol) was slowly added. After complete addition, the mixture was heated to 60° C. to react for 16 hours. The mixture was cooled to room temperature, and evaporated under reduced pressure to remove the solvent. The reaction solution was poured into 100 mL of ice water, extracted with dichloromethane, dried over anhydrous sodium sulfate, and concentrated. The crude was separated by column chromatography (PE/EA, 9/1) to obtain a5-2 (24 g) as a yellow oil, with a yield of 54%. LCMS ESI-MS m/z: 329 [M+H]⁺.

Step 2: The intermediate a5-2 (22.2 g, 67.6 mmol) from the previous step and 5-bromo-1-H-3-amino 1,2,4-triazole a1-2 (11.0 g, 67.6 mmol) were dissolved in 200 mL of ethanol, and polyphosphoric acid PPA (7.8 g, 67.6 mmol) was slowly added. After complete addition, the mixture was heated to 80° C. to react for 12 hours. The mixture was cooled to room temperature, and evaporated under reduced pressure to remove the solvent. The reaction solution was poured into 100 mL of ice water, adjusted to pH of about 8 with saturated aqueous sodium bicarbonate solution, extracted with dichloromethane, dried over anhydrous sodium sulfate, and concentrated. The crude was separated by column chromatography (PE/EA, 3/1) to obtain a5 (4.4 g) as a yellow solid, with a yield of 15%. LCMS ESI-MS m/z: 427 [M+H]⁺.

Referring to the synthetic route of intermediate a1 or a5, the following target intermediates were synthesized using similar raw materials/analogues.

| | | LC-MS |
|---|---|---|
| Intermediate | Intermediate Structure | ESI-MS m/z: [M + H]⁺ |
| a12 | 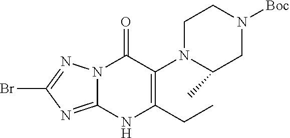 [SMILES: CCc1[nH]c2nc(Br)nn2c(=O)c1N1CCN(C(=O)OC(C)(C)C)C[C@@H]1C] | 441 |
| a13 | 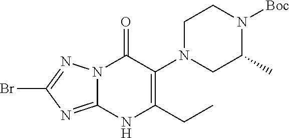 [SMILES: CCc1[nH]c2nc(Br)nn2c(=O)c1N1CCN(C(=O)OC(C)(C)C)[C@H](C)C1] | 441 |
| a14 | 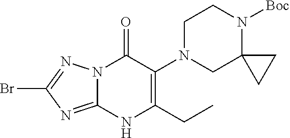 [SMILES: CCc1[nH]c2nc(Br)nn2c(=O)c1N1CCN(C(=O)OC(C)(C)C)C2(CC2)C1] | 453 |
| a15 | 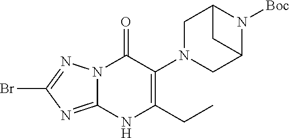 [SMILES: CCc1[nH]c2nc(Br)nn2c(=O)c1N1CC2CC(C1)N2C(=O)OC(C)(C)C] | 439 |
| a16 | 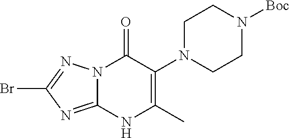 [SMILES: Cc1[nH]c2nc(Br)nn2c(=O)c1N1CCN(C(=O)OC(C)(C)C)CC1] | 413 |
| a17 | 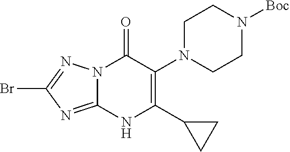 [SMILES: CC(C)(C)OC(=O)N1CCN(c2c(C3CC3)[nH]c3nc(Br)nn3c2=O)CC1] | 439 |
| a18 | 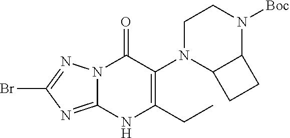 [SMILES: CCc1[nH]c2nc(Br)nn2c(=O)c1N1CCN(C(=O)OC(C)(C)C)C2CCC21] | 453 |
| a19 | 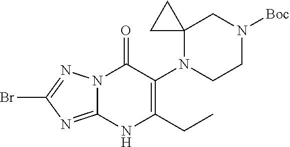 [SMILES: CCc1[nH]c2nc(Br)nn2c(=O)c1N1CCN(C(=O)OC(C)(C)C)CC12CC2] | 453 |
| a31 | 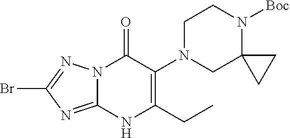 [SMILES: CCc1[nH]c2nc(Br)nn2c(=O)c1N1CCN(C(=O)OC(C)(C)C)C2(CC2)C1] | 453 |

Preparation of Intermediates a2, a6-a7, and a20-a26

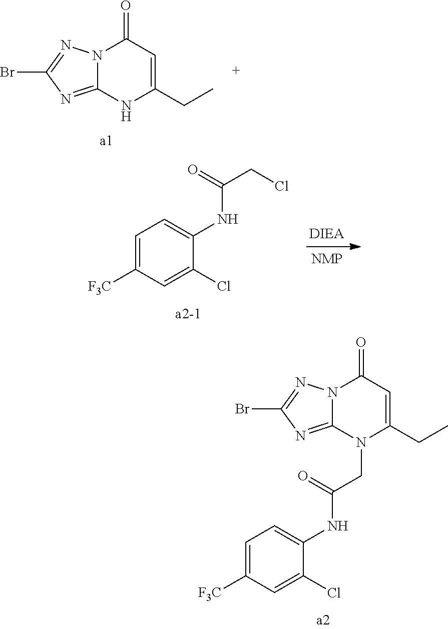 [SMILES: CCc1cc(=O)n2nc(Br)nc2[nH]1 ; O=C(CCl)Nc1ccc(C(F)(F)F)cc1Cl ; CCc1cc(=O)n2nc(Br)nc2n1CC(=O)Nc1ccc(C(F)(F)F)cc1Cl]

Procedure: the intermediate a1 (7.5 g, 30.9 mmol) from the previous step and the raw material a2-1 (10.1 g, 37.0 mmol) were dissolved in 75 mL NMP, and DIEA (12.0 g, 92.6 mmol) was slowly added. After complete addition, the mixture was heated to 50° C. to react for 16 hours, then cooled to room temperature to stop the reaction. The reaction solution was poured into 100 mL ice water, extracted with dichloromethane, dried over anhydrous sodium sulfate, and concentrated. The crude was separated by HPLC preparative chromatography (Column: XSelect Prep OBD C18 Column, 30*150 mm, 5 μm; mobile phase A: water (10 mmol/L NH₄HCO₃), mobile phase B: acetonitrile; flow rate: 90 mL/min; retention time: 3 min) to obtain a2 (5.2 g) as a yellow solid, with a yield of 35%. LCMS ESI-MS m/z: 478 [M+H]⁺.

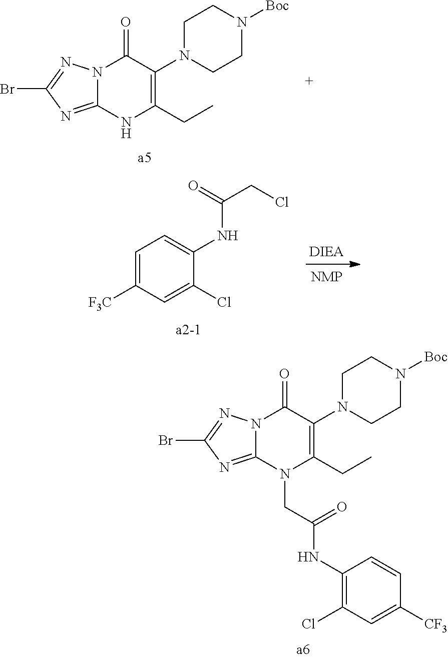 [SMILES: CCc1[nH]c2nc(Br)nn2c(=O)c1N1CCN(C(=O)OC(C)(C)C)CC1 ; O=C(CCl)Nc1ccc(C(F)(F)F)cc1Cl ; CCc1c(N2CCN(C(=O)OC(C)(C)C)CC2)c(=O)n2nc(Br)nc2n1CC(=O)Nc1ccc(C(F)(F)F)cc1Cl]

Procedure: intermediate a5 (1.1 g, 2.57 mmol) and the raw material a2-1 (1.1 g, 3.86 mmol) were dissolved in 11 mL NMP, and DIEA (1.0 g, 7.72 mmol) was slowly added. After complete addition, the mixture was heated to 70° C. to react for 12 hours, then cooled to room temperature to stop the reaction. The reaction solution was poured into 50 mL ice water, extracted with dichloromethane, dried over anhydrous sodium sulfate, and concentrated. The crude was separated by flash reverse column chromatography (chromatographic column: C18; CH₃CN/H₂O=5/1) to obtain a6 (996 mg) as a yellow solid, with a yield of 58%. LCMS ESI-MS m/z: 662 [M+H]⁺.

Referring to the synthetic route of intermediate a2 or a6, the following target intermediates were synthesized using similar raw materials/analogues.

[Similar heteroaryl NH₂ was used as raw material to react with chloroacetyl chloride to synthesize a2-1 analogue]

| | | LC-MS |
|---|---|---|
| Intermediate | Intermediate structure | ESI-MS m/z: [M + H]⁺ |
| a7 | 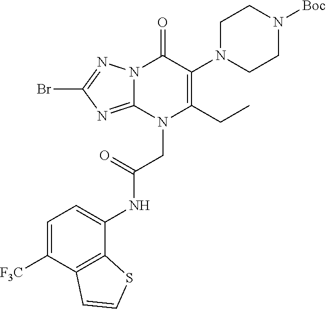 [SMILES: CCc1c(N2CCN(C(=O)OC(C)(C)C)CC2)c(=O)n2nc(Br)nc2n1CC(=O)Nc1ccc(C(F)(F)F)c2ccsc12] | 684 |
| a20 | 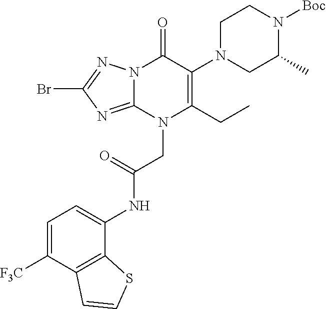 [SMILES: CCc1c(N2CCN(C(=O)OC(C)(C)C)[C@H](C)C2)c(=O)n2nc(Br)nc2n1CC(=O)Nc1ccc(C(F)(F)F)c2ccsc12] | 698 |
| a21 | 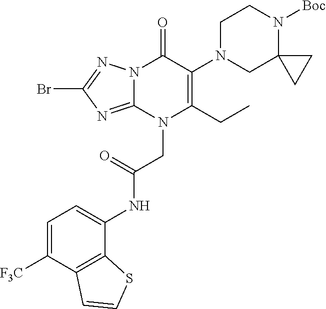 [SMILES: CCc1c(N2CCN(C(=O)OC(C)(C)C)C3(CC3)C2)c(=O)n2nc(Br)nc2n1CC(=O)Nc1ccc(C(F)(F)F)c2ccsc12] | 710 |
| a22 | 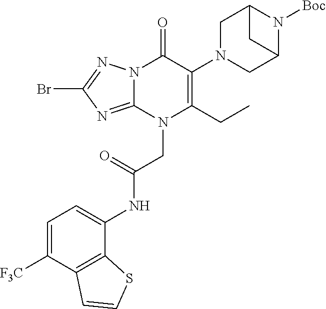 [SMILES: CCc1c(N2CC3CC(C2)N3C(=O)OC(C)(C)C)c(=O)n2nc(Br)nc2n1CC(=O)Nc1ccc(C(F)(F)F)c2ccsc12] | 696 |
| a23 | 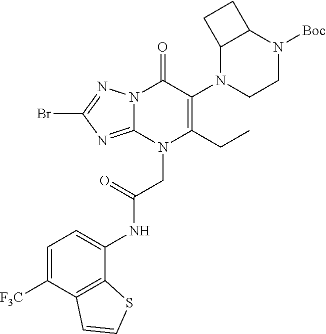 [SMILES: CCc1c(N2CCN(C(=O)OC(C)(C)C)C3CCC32)c(=O)n2nc(Br)nc2n1CC(=O)Nc1ccc(C(F)(F)F)c2ccsc12] | 710 |
| a24 | 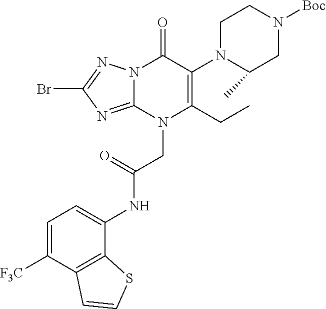 [SMILES: CCc1c(N2CCN(C(=O)OC(C)(C)C)C[C@@H]2C)c(=O)n2nc(Br)nc2n1CC(=O)Nc1ccc(C(F)(F)F)c2ccsc12] | 698 |
| a25 | 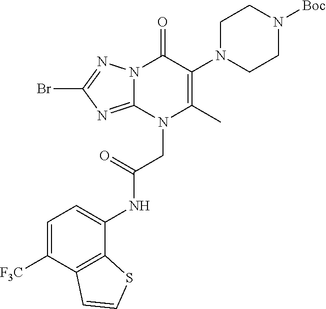 [SMILES: Cc1c(N2CCN(C(=O)OC(C)(C)C)CC2)c(=O)n2nc(Br)nc2n1CC(=O)Nc1ccc(C(F)(F)F)c2ccsc12] | 670 |
| a26 | 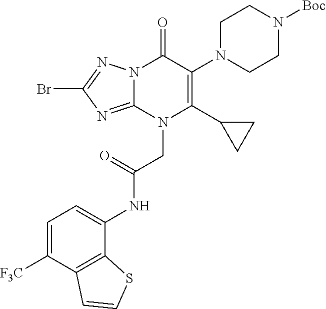 [SMILES: CC(C)(C)OC(=O)N1CCN(c2c(C3CC3)n(CC(=O)Nc3ccc(C(F)(F)F)c4ccsc34)c3nc(Br)nn3c2=O)CC1] | 696 |

Preparation of Intermediates a3, a8-a9, and a27-a30

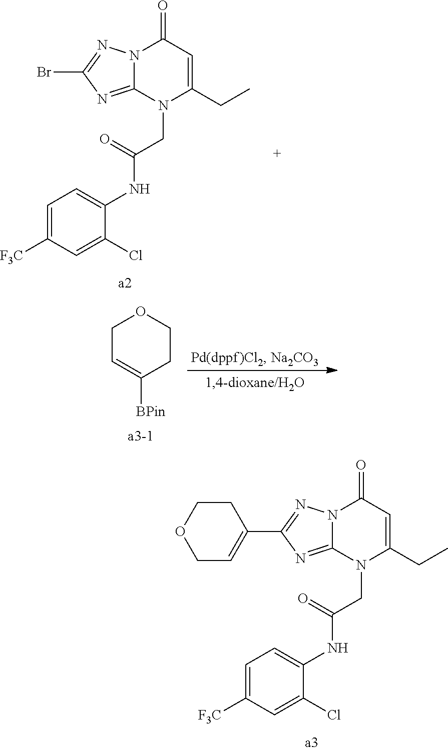

Procedure: Under nitrogen, intermediate a2 (5.0 g, 10.4 mmol), the raw material a3-1 (2.9 g, 13.6 mmol) and sodium carbonate (3.3 g, 31.3 mmol) were dissolved in 50 mL of a mixed solution of 1,4-dioxane and water (v/v, 4/1), and catalyst Pd(dppf)Cl₂ (900 mg, 1.0 mmol) was added. The mixture was heated to 100° C. to react for 2 hours. The reaction was stopped, and the mixture was filtered. 100 mL of water was added to the reaction solution. The mixture was extracted with ethyl acetate, dried over anhydrous sodium sulfate, and concentrated. The crude was separated by HPLC preparative chromatography (Column: WelFlash C18-I, 20-40 μm, 330 g; mobile phase A water:(10 mmol/L NH₄HCO₃), mobile phase B: acetonitrile; flow rate: 80 mL/min; retention time: 12 min) to obtain a3 (2.6 g) as a yellow solid, with a yield of 52%. LCMS ESI-MS m/z: 482 [M+H]⁺.

Referring to the synthetic route of intermediate a3, the following target intermediates were synthesized using similar raw materials/analogues.

| | | LC-MS |
|---|---|---|
| Intermediate | Intermediate structure | ESI-MS m/z: [M + H]⁺ |
| a8 | 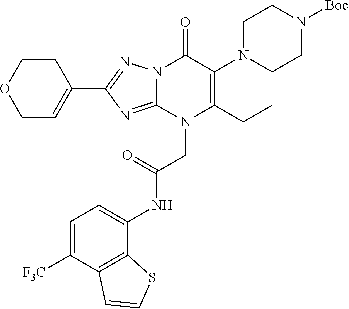 [SMILES: CCc1c(N2CCN(C(=O)OC(C)(C)C)CC2)c(=O)n2nc(C3=CCOCC3)nc2n1CC(=O)Nc1ccc(C(F)(F)F)c2ccsc12] | 688 |
| a9 | 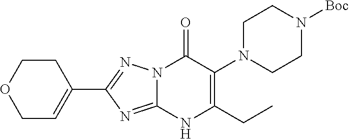 [SMILES: CCc1[nH]c2nc(C3=CCOCC3)nn2c(=O)c1N1CCN(C(=O)OC(C)(C)C)CC1] | 431 |
| a27 | 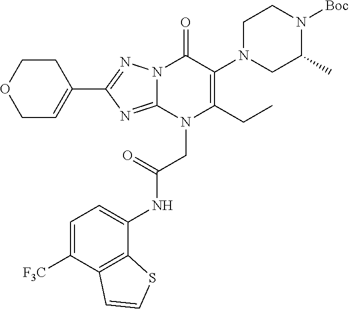 [SMILES: CCc1c(N2CCN(C(=O)OC(C)(C)C)[C@H](C)C2)c(=O)n2nc(C3=CCOCC3)nc2n1CC(=O)Nc1ccc(C(F)(F)F)c2ccsc12] | 702 |
| a28 | 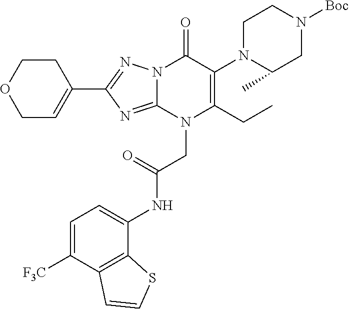 [SMILES: CCc1c(N2CCN(C(=O)OC(C)(C)C)C[C@@H]2C)c(=O)n2nc(C3=CCOCC3)nc2n1CC(=O)Nc1ccc(C(F)(F)F)c2ccsc12] | 702 |
| a29 | 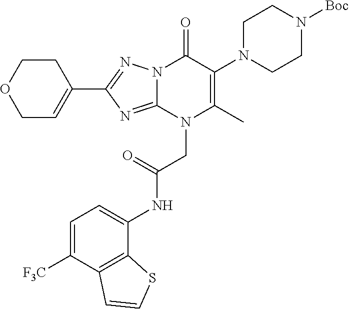 [SMILES: Cc1c(N2CCN(C(=O)OC(C)(C)C)CC2)c(=O)n2nc(C3=CCOCC3)nc2n1CC(=O)Nc1ccc(C(F)(F)F)c2ccsc12] | 674 |
| a30 | 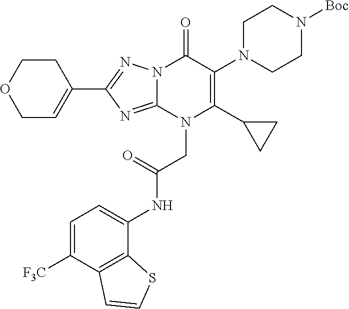 [SMILES: CC(C)(C)OC(=O)N1CCN(c2c(C3CC3)n(CC(=O)Nc3ccc(C(F)(F)F)c4ccsc34)c3nc(C4=CCOCC4)nn3c2=O)CC1] | 700 |

### Preparation of Intermediate a4

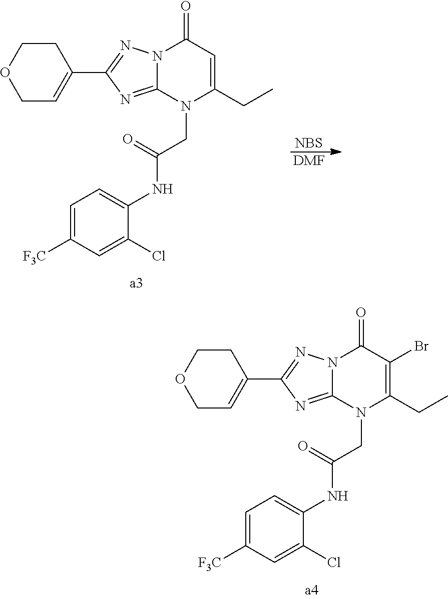

Procedure: intermediate a3 (2.4 g, 5.0 mmol) was dissolved in 25 mL DMF, and NBS (1.8 g, 10.0 mmol) was added. The mixture was reacted at room temperature for 2 hours. The reaction was stopped, and the mixture was filtered. 100 mL of water was added to the reaction solution. The mixture was extracted with ethyl acetate, dried over anhydrous sodium sulfate, and concentrated. The crude was separated by HPLC preparative chromatography (Column: WelFlash C18-I, 20-40 μm, 330 g; mobile phase A water:(10 mmol/L NH₄HCO₃), mobile phase B: acetonitrile; flow rate: 90 mL/min; retention time: 11 min) to obtain a4 (1.6 g) as a white solid, with a yield of 57%. LCMS ESI-MS m/z: 560 [M+H]⁺.

Preparation of Intermediates a10-a11

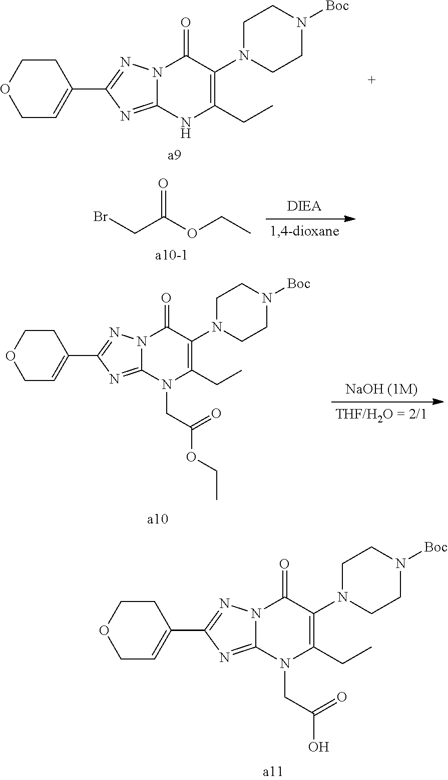

Step 1: Under nitrogen protection, intermediate a9 (1.5 g, 3.48 mmol) and DIEA (1.4 g, 10.5 mmol) were dissolved in 15 mL 1,4-dioxane, and the raw material a10-1 (0.8 g, 4.88 mmol) was added. The mixture was heated to 85° C. to react for 4 hours. The reaction was stopped. 100 mL of water was added to the reaction solution. The mixture was extracted with ethyl acetate, dried over anhydrous sodium sulfate, and concentrated. The crude was separated by flash reverse column chromatography (Column: C18; CH₃CN/H₂O, 5/4) to obtain a10 (1.4 g) as a yellow solid. yield: 78%. LCMS ESI-MS m/z: 517 [M+H]⁺.

Step 2: intermediate a10 (1.2 g, 2.32 mmol) was dissolved in 18 mL of a mixed solution of tetrahydrofuran and water (v/v, 2/1), and aqueous NaOH solution (3.5 mL, 1N) was added to the reaction solution. The mixture was reacted at room temperature for 1 hour and evaporated under reduced pressure to remove the solvent. The mixture was adjusted to pH of about 4 with dilute hydrochloric acid and evaporated under reduced pressure to remove the solvent. The crude was separated by flash reverse column chromatography (Column: C18; CH₃CN/H₂O, 1/1) to obtain all (900 mg) as a yellow solid, with a yield of 92%. LCMS ESI-MS m/z: 489 [M+H]⁺.

### Preparation of Intermediate b1

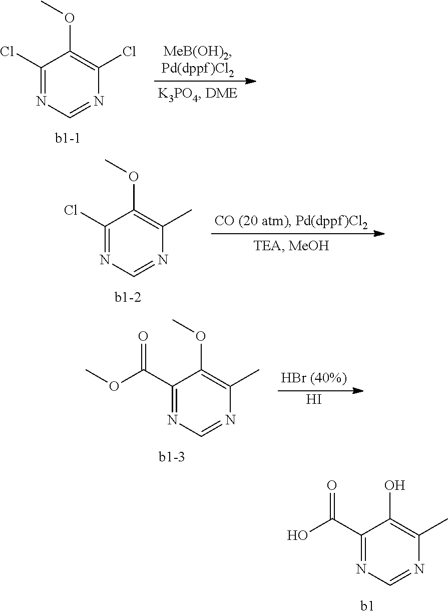

Step 1: Under nitrogen, the raw material 4,6-dichloro-5-methoxypyrimidine b1-1 (20.0 g, 111 mmol), raw material methylboric acid (7.0 g, 117 mmol) and potassium phosphate (59.2 g, 279 mmol) were dissolved in 120 mL DME, and catalyst Pd(dppf)Cl₂ (4.6 g, 5.6 mmol) was added. The mixture was heated to 85° C. to react for 12 hours. The reaction was stopped, and the mixture was filtered. 100 mL of water was added to the reaction solution. The mixture was extracted with ethyl acetate, dried over anhydrous sodium sulfate, and concentrated. The crude was separated by column chromatography (PE/EA, 10/1) to obtain b1-2 (6.0 g) as a white solid, with a yield of 34%. LCMS ESI-MS m/z: 159 [M+H]⁺.

Step 2: Under carbon monoxide atmosphere, the intermediate b1-2 (6.0 g, 37.0 mmol) from the previous step and TEA (7.66 g, 75.0 mmol) were dissolved in 90 mL of methanol, and catalyst Pd(dppf) Cl₂(1.85 g, 2.3 mmol) was added. The mixture was heated to 100° C. under CO (20 atm) to react for 12 hours. The reaction was stopped, and the mixture was filtered. The solvent was removed by evaporation under reduced pressure, and the crude product was separated by HPLC preparative chromatography (Column: WelFlash C18-I, 20-40 um, 120 g; mobile phase A: water (10 mmol/L NH₄HCO₃) mobile phase B: acetonitrile; flow rate: 60 mL/min) to obtain b1-3 (5.0 g) as a white solid, with a yield of 73%. LCMS ESI-MS m/z: 183 [M+H]⁺.

Step 3: the intermediate b1-3 (3.0 g, 16.5 mmol) from the previous step was dissolved in 15 mL aqueous HBr solution (40%), and the solution was heated to 40° C. to react for 10 hours. The reaction was stopped. HI (15 mL) was added to the reaction solution. The mixture was reacted at 40° C. for another 6 hours, and evaporated under reduced pressure to remove the solvent. The crude product was adjusted to about pH 8 with aqueous NaOH solution (1N), and then adjusted to about pH 3 with concentrated hydrochloric acid. The mixture was evaporated under reduced pressure to remove the solvent. The crude product was separated by flash reverse column chromatography (Column: C18; CH₃CN/H₂O, 1/1) to give b1 (1.5 g) as a yellow solid, with a yield of 59%. LCMS ESI-MS m/z: 155 [M+H]⁺.

Preparation of Intermediates b2-b3

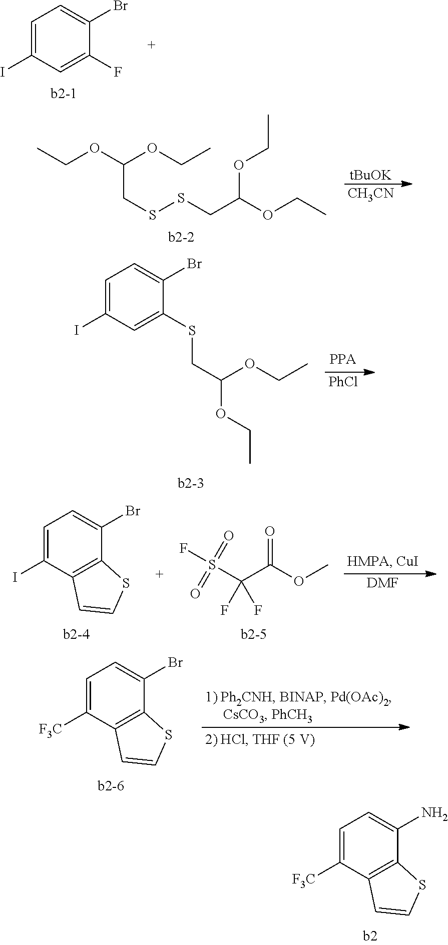

Step 1: Under nitrogen, the raw material 1-bromo-2-fluoro-4-iodobenzene b2-1 (1.95 g, 6.5 mmol) and raw material b2-2 (2.32 g, 7.8 mmol) were dissolved in 40 mL acetonitrile, and potassium tert-butoxide (2.2 g, 19.4 mmol) was added. The mixture was heated to 50° C. to react for 6 hours. The reaction was stopped, and the mixture was filtered. 100 mL of water was added to the reaction solution. The mixture was extracted with ethyl acetate, dried over anhydrous sodium sulfate, and concentrated. The crude was separated by column chromatography (PE/EA, 20/1) to obtain b2-3 (2.22 g) as a yellow oil, with a yield of 79%.

Step 2: the intermediate b2-3 (2.22 g, 5.15 mmol) from the previous step was dissolved in 133 mL of chlorobenzene, and polyphosphoric acid PPA (2.37 g, 20.6 mmol) was added. The mixture was heated to 130° C. to react for 5 hours. The reaction was stopped, and the mixture was cooled to room temperature. 100 mL of ice water was added to the reaction solution. The mixture was extracted with ethyl acetate, dried over anhydrous sodium sulfate, and concentrated. The crude was separated by flash reverse column chromatography (Column: C18; CH₃CN/H₂O, 2/1) to obtain b2-4 (1.05 g) as a yellow oil, with a yield of 60%.

Step 3: Under nitrogen, the intermediate b2-4 (1.05 g, 3.1 mmol) from the previous step and the raw material b2-5 (1.95 g, 9.3 mmol) were dissolved in 21 mL of DMF, and catalyst CuI (290 mg, 1.55 mmol) and HMPA (2.39 g, 13.3 mmol) were added. The mixture was heated to 100° C. to react for 2 hours. The reaction was stopped, and the mixture was filtered. 60 mL of water was added to the reaction solution, and the mixture was extracted with methyl tert-butyl ether, dried over anhydrous sodium sulfate, and concentrated. The crude was separated by column chromatography (PE/EA, 100/1) to obtain b2-6 (0.8 g) as a yellow solid, with a yield of 92%.

Step 4: Under nitrogen, the intermediate b2-6 (0.8 g, 2.85 mmol) from the previous step and the raw material benzophenonimine (1.03 g, 5.69 mmol) were dissolved in 16 mL of toluene, and the catalyst Pd(OAc)₂ (60 mg, 0.28 mmol), the ligand BINAP (0.35 g, 0.57 mmol) and cesium carbonate (1.85 g, 5.69 mmol) were added. The mixture was heated to 110° C. to react for 2 hours. The reaction was stopped, and the mixture was filtered. 60 mL of water was added to the reaction solution. The mixture was extracted with ethyl acetate, dried over anhydrous sodium sulfate, and concentrated to obtain a crude mixture. The mixture was dissolved in 4 mL tetrahydrofuran, and a 2M solution of hydrogen chloride in tetrahydrofuran (16 mL) was added. The mixture was reacted at room temperature for 1 hour, and the reaction was stopped. Saturated aqueous sodium bicarbonate solution was added to the reaction solution to adjust the pH to about 8. The mixture was extracted with ethyl acetate, dried over anhydrous sodium sulfate, and concentrated. The crude was separated by flash reverse column chromatography (Column: C18; CH₃CN/H₂O, 10/7) to obtain b2 (0.5 g) as a yellow solid, with a yield of 81%. LCMS ESI-MS m/z: 218 [M+H]⁺.

Referring to the synthetic route of intermediate b2, the following target intermediates were synthesized using similar raw materials/analogues.

| | | LC-MS |
|---|---|---|
| Intermediate | Intermediate structure | ESI-MS m/z: [M + H]⁺ |
| b3 | 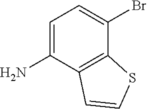 [SMILES: Nc1ccc(Br)c2sccc12] | 218 |

### Preparation of Intermediates b4 and b6

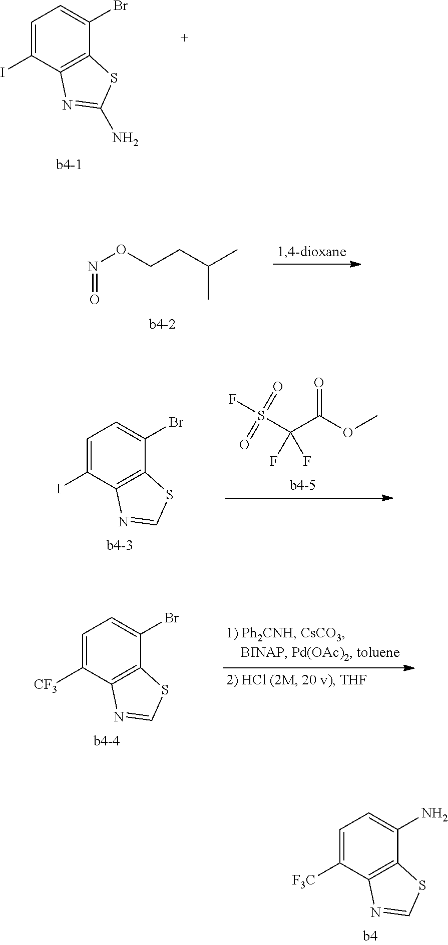

Step 1: Under nitrogen, the raw material 7-bromo-4-iodo-1,3-benzothiazol-2-amine b4-1 (3.4 g, 9.57 mmol) was dissolved in 68 mL 1,4-dioxane, and isopentyl nitrite b4-2 (1.6 g, 14.4 mmol) was added dropwise. The mixture was heated to 60° C. to react for 1 hour. The reaction was stopped, and the mixture was filtered. 150 mL of water was added to the reaction solution. The mixture was extracted with ethyl acetate, dried over anhydrous sodium sulfate, and concentrated. The crude was separated by column chromatography (PE/EA, 10/1) to obtain yellow solid b4-3 (2.2 g), with a yield of 68%. LCMS ESI-MS m/z: 340 [M+H]⁺.

Step 2: Under nitrogen, the intermediate b4-3 (2.2 g, 6.47 mmol) from the previous step and the raw material b2-5 (3.7 g, 19.4 mmol) were dissolved in 44 mL DMF, and catalyst CuI (1.0 g, 5.2 mmol) and HMPA (4.9 g, 27.8 mmol) were added. The mixture was heated to 100° C. to react for 2 hours. The reaction was stopped, and the mixture was filtered. 60 mL of water was added to the reaction solution. The mixture was extracted with methyl tert-butyl ether, dried over anhydrous sodium sulfate, and concentrated. The crude was separated by flash reverse column chromatography (Column: C18; CH₃CN/H₂O, 1/1) to give b4-4 (1.5 g) as a yellow solid, with a yield of 82%. LCMS ESI-MS m/z: 282 [M+H]⁺.

Step 3: Under nitrogen, the intermediate b4-4 (1.5 g, 5.33 mmol) from the previous step and the raw material benzophenonimine (2.9 g, 16.0 mmol) were dissolved in 30 mL of toluene, and the catalyst Pd(OAc)₂(200 mg, 1.06 mmol), the ligand BINAP (1.0 g, 1.6 mmol) and cesium carbonate (5.2 g, 16.0 mmol) were added. The mixture was heated to 100° C. to react for 12 hours. The reaction was stopped, and the mixture was filtered. 100 mL of water was added to the reaction solution. The mixture was extracted with ethyl acetate, dried over anhydrous sodium sulfate, and concentrated to obtain a crude mixture. The mixture was dissolved in 4 mL tetrahydrofuran, and a 2M solution of hydrogen chloride in tetrahydrofuran (30 mL) was added. The mixture was reacted at room temperature for 1 hour, and then the reaction was stopped. Saturated aqueous sodium bicarbonate solution was added to the reaction solution to adjust the pH to about 8. The mixture was extracted with ethyl acetate, dried over anhydrous sodium sulfate, and concentrated. The crude was separated by flash reverse column chromatography (Column: C18; CH₃CN/H₂O, 1/1) to give b4 (660 mg) as a yellow solid, with a yield of 57%. LCMS ESI-MS m/z: 219 [M+H]⁺.

Referring to the synthetic route of intermediate b4, the following target intermediates were synthesized using similar raw materials/analogues.

| | | LC-MS |
|---|---|---|
| Intermediate | Intermediate structure | ESI-MS m/z: [M + H]⁺ |
| b6 | 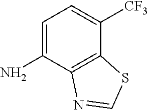 [SMILES: Nc1ccc(C(F)(F)F)c2scnc12] | 219 |

### Preparation of Intermediate b5

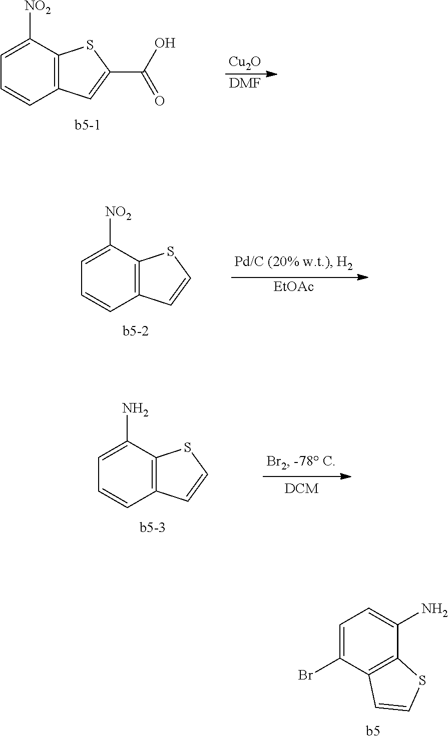

Step 1: The raw material 7-nitrobenzo[b]thiophene-2-carboxylic acid b5-1 (900 mg, 4.03 mmol) and Cu₂O (115 mg, 0.81 mmol) were dissolved in 18 mL of DMF. The mixture was heated to 150° C. and reacted for 12 hours. The reaction was stopped, and the mixture was filtered. 150 mL of water was added to the reaction solution, and the mixture was extracted with ethyl acetate, washed with saturated brine, dried over anhydrous sodium sulfate, and concentrated. The crude was separated by flash reverse column chromatography (Column: C18; CH₃CN/H₂O, 4/3) to obtain b5-2 (700 mg) as a yellow solid, with a yield of 97%. LCMS ESI-MS m/z: 180 [M+H]⁺.

Step 2: Under hydrogen conditions (4atm), the intermediate b5-2 (700 mg, 3.91 mmol) from the previous step was dissolved in 14 mL of ethyl acetate, and catalyst Pd/C (140 mg) was added. The mixture was reacted at room temperature for 6 hours. The reaction was stopped, and the mixture was filtered to obtain b5-3 (600 mg) as a yellow oil. LCMS ESI-MS m/z: 150 [M+H]⁺.

Step 3: Under nitrogen, at-78° C., the intermediate b5-3 (600 g, 4.02 mmol) from the previous step was dissolved in 12 mL of dichloromethane, and liquid Br₂ (643 mg, 4.02 mmol) was added. The mixture was reacted at this temperature for 1 hour, and then the reaction was stopped. 100 mL of ice water was added to the reaction solution, and the mixture was extracted with dichloromethane, dried over anhydrous sodium sulfate, and concentrated. The crude was separated by flash reverse column chromatography (Column: C18; CH₃CN/H₂O, 5/4) to give b5 (300 mg) as a yellow solid, two-step yield: 33%. LCMS ESI-MS m/z: 228 [M+H]⁺.

Preparation of Intermediates c1-c4

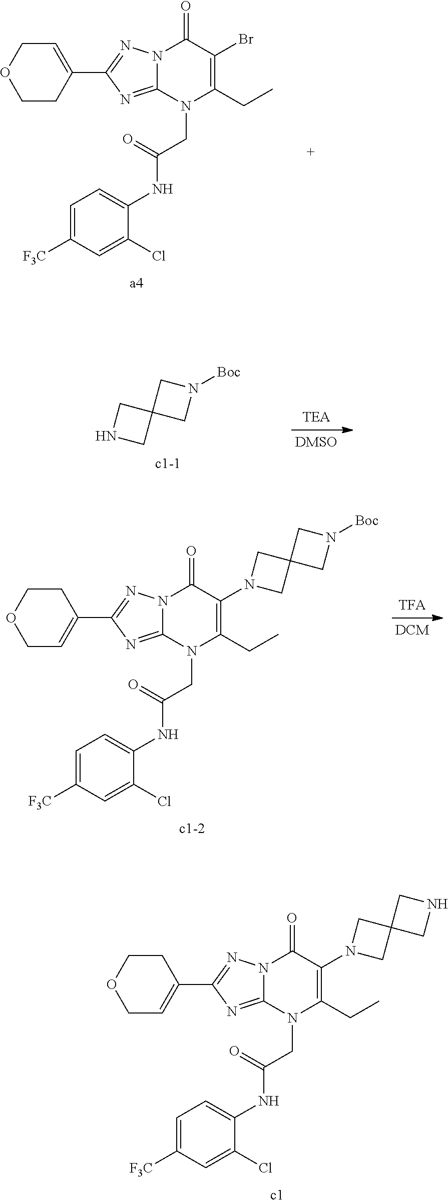 [SMILES: CCc1c(Br)c(=O)n2nc(C3=CCOCC3)nc2n1CC(=O)Nc1ccc(C(F)(F)F)cc1Cl ; CC(C)(C)OC(=O)N1CC2(CNC2)C1 ; CCc1c(N2CC3(CN(C(=O)OC(C)(C)C)C3)C2)c(=O)n2nc(C3=CCOCC3)nc2n1CC(=O)Nc1ccc(C(F)(F)F)cc1Cl ; CCc1c(N2CC3(CNC3)C2)c(=O)n2nc(C3=CCOCC3)nc2n1CC(=O)Nc1ccc(C(F)(F)F)cc1Cl]

Step 1: Under nitrogen, intermediate a4 (210 mg, 0.3 mmol) and TEA (114 mg, 1.1 mmol) were dissolved in 2 mL DMSO, and the raw material c1-1 (371 mg, 1.9 mmol) was added. The mixture was heated to 85° C. to react for 12 hours, and then the reaction was stopped. 100 mL of water was added to the reaction solution. The mixture was extracted with ethyl acetate, dried over anhydrous sodium sulfate, and concentrated. The crude was separated by HPLC chromatography (Column: WelFlash C18-I, 20-40 μm, 180 g; mobile phase A: water (10 mmol/L NH₄HCO₃), mobile phase B: acetonitrile; flow rate: 70 mL/min; retention time: 12 min) to obtain ci-2 (120 mg) as a white solid, with a yield of 47%. LCMS ESI-MS m/z: 678 [M+H]⁺.

Step 2: the intermediate c1-2 (120 mg, 0.2 mmol) from the previous step and trifluoroacetic acid (0.5 mL) were dissolved in 2 mL of dichloromethane, and the mixture was reacted at room temperature for 2 hours. The reaction was stopped. The solvent was removed by evaporation under reduced pressure, and the crude product was separated by HPLC preparative chromatography (Column: WelFlash C18-I, 20-40 um, 40 g; mobile phase A: water (10 mmol/L NH₄HCO₃) mobile phase B: acetonitrile; flow rate: 50 mL/min; retention time: 9 min) to give c1 (80 mg) as a white solid, with a yield of 78%. LCMS ESI-MS m/z: 578 [M+H]⁺.

Referring to the synthetic route of intermediate c1, the following target intermediates were

| | | LC-MS |
|---|---|---|
| Intermediate | Intermediate structure | ESI-MS m/z: [M + H]⁺ |
| c2 | 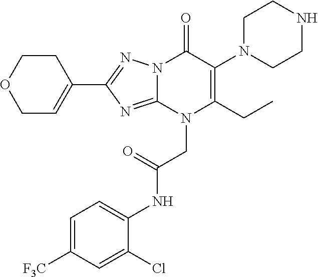 [SMILES: CCc1c(N2CCNCC2)c(=O)n2nc(C3=CCOCC3)nc2n1CC(=O)Nc1ccc(C(F)(F)F)cc1Cl] | 566 |
| c3 | 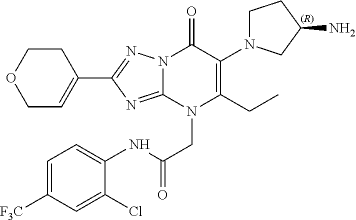 | 566 |
| c4 | 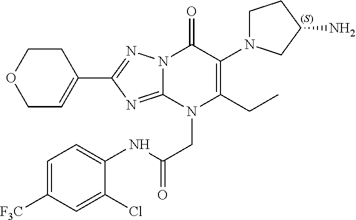 | 566 |

Preparation of Intermediates c5-c8

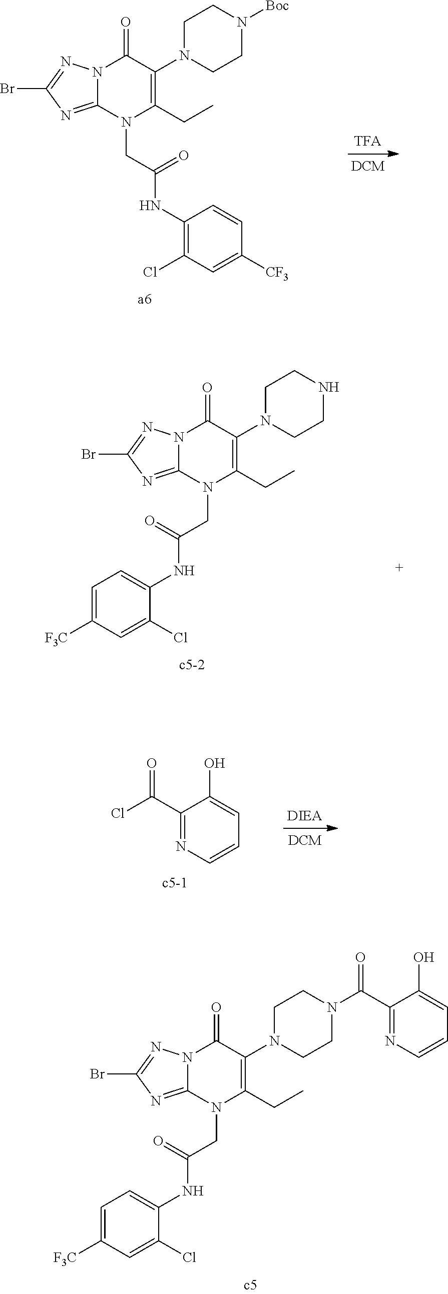 [SMILES: CCc1c(N2CCN(C(=O)OC(C)(C)C)CC2)c(=O)n2nc(Br)nc2n1CC(=O)Nc1ccc(C(F)(F)F)cc1Cl ; CCc1c(N2CCNCC2)c(=O)n2nc(Br)nc2n1CC(=O)Nc1ccc(C(F)(F)F)cc1Cl ; O=C(Cl)c1ncccc1O ; CCc1c(N2CCN(C(=O)c3ncccc3O)CC2)c(=O)n2nc(Br)nc2n1CC(=O)Nc1ccc(C(F)(F)F)cc1Cl]

Step 1: Intermediate a6 (996 mg, 1.5 mmol) was dissolved in 10 mL of dichloromethane, and 0.5 mL of trifluoroacetic acid was added dropwise. The mixture was reacted at room temperature for 1 hour, and the reaction was stopped. 20 mL of water was added to the reaction solution, and the mixture was adjusted to pH of about 9 with saturated aqueous sodium bicarbonate solution. The mixture was extracted with dichloromethane, dried over anhydrous sodium sulfate, and concentrated to obtain c5-2 (694 mg) as a yellow solid, with a yield of 82%. LCMS ESI-MS m/z: 562 [M+H]⁺.

Step 2: the intermediate c5-2 (494 mg, 0.88 mmol) from the previous step and DIEA(567 mg, 4.4 mmol) were dissolved in 5 mL of dichloromethane, and the raw material 3-hydroxy-2-pyridinecarbonyl chloride c5-1 (277 mg, 1.76 mmol) was added. The mixture was reacted at room temperature for another 2 hours, and the reaction was stopped. The solvent was removed by evaporation under reduced pressure, and the crude product was separated by flash column chromatography (Column: WelFlash C18-I, 20-40 um, 130 g; mobile phase A: water, mobile phase B: acetonitrile; flow rate: 60 mL/min; retention time: 14 min) to obtain c5 (360 mg) as a yellow solid, with a yield of 60%. LCMS ESI-MS m/z: 683 [M+H]⁺.

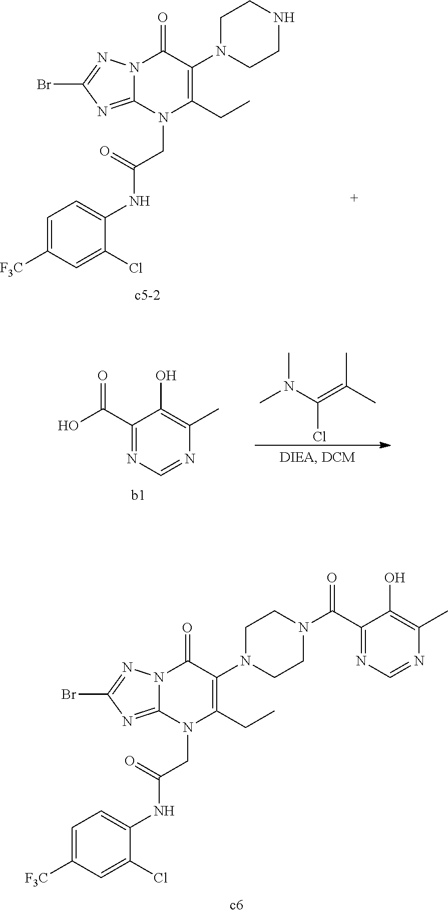

Procedure: In an ice bath, under nitrogen, intermediate b1 (49 mg, 0.32 mmol) and 1-chloro-N,N,2-trimethylpropyl-1-en-1-amine (43 mg, 0.32 mmol) were dissolved in 2 mL of dichloromethane, and the mixture was stirred for 1 hour under ice bath. DIEA (138 mg, 1.06 mmol) and intermediate c5-2 (120 mg, 0.21 mmol) were added to the reaction solution. The mixture was reacted at room temperature for 1 hour, and the reaction was stopped. 15 mL of water was added to the reaction solution, and the mixture was extracted with dichloromethane, dried over anhydrous sodium sulfate, and concentrated. The crude was separated by flash reverse column chromatography (Column: C18; CH₃CN/H₂O, 4/5) to obtain c6 (45 mg) as a light-yellow solid, with a yield of 30%. LCMS ESI-MS m/z: 698 [M+H]⁺.

Referring to the synthetic route of intermediate c5 or c6, the following target intermediates were synthesized using similar raw materials/analogues.

| | | LC-MS |
|---|---|---|
| Intermediate | Intermediate structure | ESI-MS m/z: [M + H]⁺ |
| c7 | 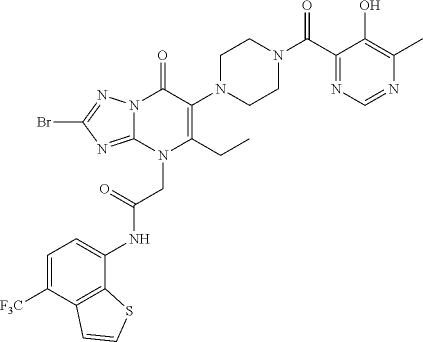 [SMILES: CCc1c(N2CCN(C(=O)c3ncnc(C)c3O)CC2)c(=O)n2nc(Br)nc2n1CC(=O)Nc1ccc(C(F)(F)F)c2ccsc12] | 720 |
| c8 | 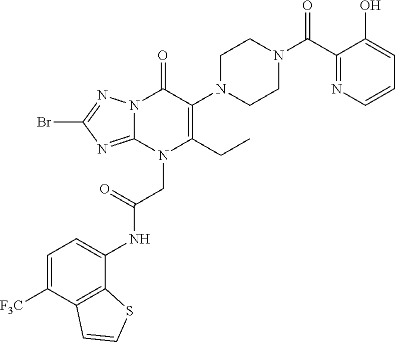 [SMILES: CCc1c(N2CCN(C(=O)c3ncccc3O)CC2)c(=O)n2nc(Br)nc2n1CC(=O)Nc1ccc(C(F)(F)F)c2ccsc12] | 705 |

Preparation of Intermediates d1-d2

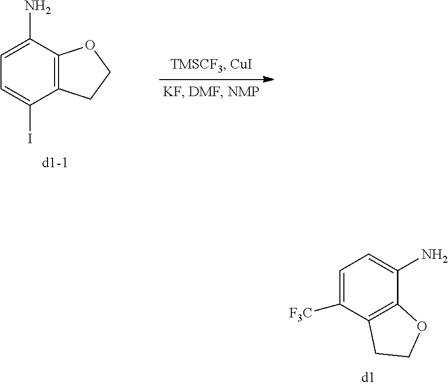 [SMILES: Nc1ccc(I)c2c1OCC2 ; Nc1ccc(C(F)(F)F)c2c1OCC2]

Procedure: Under nitrogen, TMSCF₃ (490 mg, 3.44 mmol) and KF (200 mg, 3.44 mmol) were dissolved in 12 mL of a mixed solution of DMF and NMP (v/v, 1/1), and catalyst CuI (657 mg, 3.44 mmol) was added. The mixture was stirred at room temperature for 3 hours. The raw material d1-1 (600 mg, 2.29 mmol) was added to the reaction solution. The mixture was heated to 70° C. to react for 12 hours. The reaction was stopped, and the mixture was filtered. 60 mL of water was added to the reaction solution, and the mixture was extracted with dichloromethane, dried over anhydrous sodium sulfate, and concentrated. The crude was separated by flash reverse column chromatography (Column: C18; CH₃CN/H₂O, 1/1) to give d1 (150 mg) as a brown oil, with a yield of 32%. LCMS ESI-MS m/z: 204 [M+H]⁺.

Referring to the synthetic route of intermediate d1, the following target intermediates were synthesized using similar raw materials/analogues.

| | | LC-MS |
|---|---|---|
| Intermediate | Intermediate structure | ESI-MS m/z: [M + H]⁺ |
| d2 | 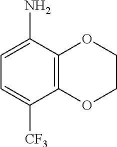 [SMILES: Nc1ccc(C(F)(F)F)c2c1OCCO2] | 220 |

### Preparation of Intermediates d3, d7

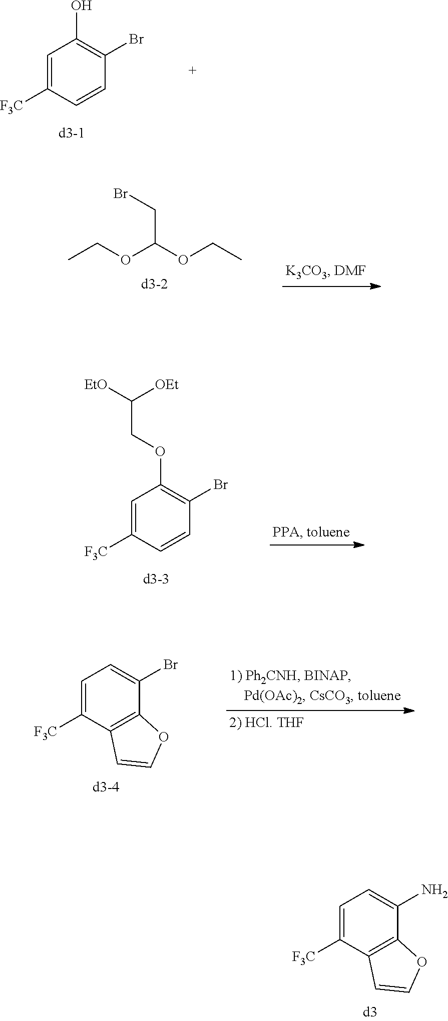

Step 1: Under nitrogen, the raw material 2-bromo-5-trifluoromethylphenol d3-1 (5.0 g, 20.75 mmol) and potassium carbonate (8.6 g, 62.2 mmol) were dissolved in 100 mL of DMF. The raw material d3-2 (12.3 g, 62.2 mmol) was added dropwise, and the mixture was heated to 80° C. to react for 12 hours. The reaction was stopped, and the mixture was filtered. 250 mL of water was added to the reaction solution. The mixture was extracted with ethyl acetate, dried over anhydrous sodium sulfate, and concentrated. The crude was separated by column chromatography (PE/EA, 20/1) to obtain d3-3 (6.0 g) as a yellow oil, with a yield of 81%.

Step 2: The intermediate d3-3 (6.0 g, 16.8 mmol) from the previous step and polyphosphoric acid (30 g, 261 mmol) were dissolved in 72 mL of toluene. The mixture was heated to 120° C. to react for 5 hours, and then the reaction was stopped. 100 mL of water was added to the reaction solution, and the pH was adjusted to about 8 with ammonia water. The mixture was extracted with methyl tert-butyl ether, dried over anhydrous sodium sulfate, concentrated, and separated by column chromatography (PE/EA, 20/1) to obtain d3-4 (1.0 g) as a yellow oil, with a yield of 23%.

Step 3: Under nitrogen, the intermediate d3-4 (1.0 g, 3.77 mmol) from the previous step and the raw material benzophenonimine (1.4 g, 7.55 mmol) were dissolved in 20 mL of toluene. Catalyst Pd(OAc)₂ (80 mg, 0.38 mmol), the ligand BINAP (0.5 g, 0.8 mmol) and cesium carbonate (2.5 g, 7.55 mmol) were added. The mixture was heated to 110° C. to react for 2 hours. The reaction was stopped, and the mixture was filtered. 100 mL of water was added to the reaction solution. The mixture was extracted with ethyl acetate, dried over anhydrous sodium sulfate, and concentrated to obtain a crude mixture. The mixture was dissolved in 5 mL of tetrahydrofuran, and a 2M solution of hydrogen chloride in tetrahydrofuran (20 mL) was added. The mixture was reacted at room temperature for 1 hour and the reaction was stopped. Saturated aqueous sodium bicarbonate solution was added to the reaction solution to adjust the pH to about 8. The mixture was extracted with ethyl acetate, dried over anhydrous sodium sulfate, and concentrated. The crude product was separated by flash reverse column chromatography (Column: C18; CH₃CN/H₂O, 1/1) to give d3 (150 mg) as a yellow solid, with a yield of 20%. LCMS ESI-MS m/z: 202 [M+H]⁺.

Referring to the synthetic route of intermediate d3, the following target intermediates were synthesized using similar raw materials/analogues.

| | | LC-MS |
|---|---|---|
| Intermediate | Intermediate structure | ESI-MS m/z: [M + H]⁺ |
| d7 | 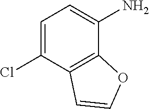 [SMILES: Nc1ccc(Cl)c2ccoc12] | 168 |

### Preparation of Intermediate d4

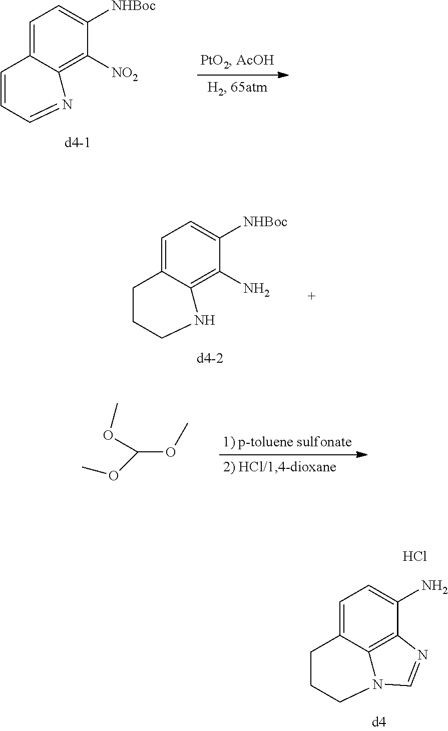

Step 1: Under hydrogen (65 atm), the raw material d4-1 (1.5 g, 5.18 mmol) was dissolved in 30 mL of acetic acid and PtO₂ (0.02 g, 0.1 mmol) was added. The mixture was reacted at room temperature for 12 hours. The reaction was stopped and the mixture was filtered. 150 mL of ammonia water was added to the reaction solution. The mixture was extracted with dichloromethane, dried over anhydrous sodium sulfate, and concentrated. The crude was separated by flash reverse column chromatography (Column: C18; CH₃CN/H₂O, 5/2) to give d4-2 (780 mg) as a yellow solid, with a yield of 57%. LCMS ESI-MS m/z: 264 [M+H]⁺.

Step 2: The intermediate d4-2 (780 mg, 2.96 mmol) from the previous step was dissolved in 8 mL of trimethyl orthoformate, and 4-methylbenzenesulfonic acid (102 mg, 0.59 mmol) was slowly added. The mixture was heated to 100° C. to react for 1 hour, and the reaction was stopped. 60 mL of water was added to the reaction solution. The mixture was extracted with ethyl acetate, dried over anhydrous sodium sulfate, and concentrated. The crude was separated by flash reverse column chromatography (Column: C18; CH₃CN/H₂O, 5/2) to obtain a yellow solid. The yellow solid was dissolved in 5 mL of a4M solution of hydrogen chloride in 1,4-dioxane. The solution was stirred at room temperature for 1 hour, and filtered to obtain d4 (570 mg) as a white solid, with a yield of 92%. LCMS ESI-MS m/z: 174 [M+H]⁺.

### Preparation of Intermediate d5

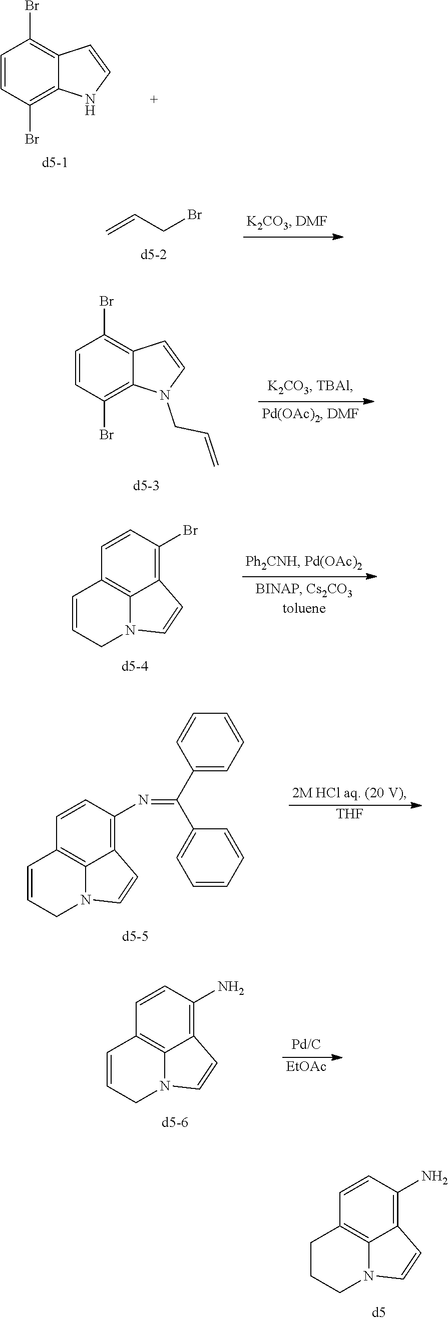

Step 1: Under nitrogen, the raw material 4,7-dibromo-1H-indole d5-1 (8.4 g, 30.55 mmol) and potassium carbonate (2.5 g, 45.8 mmol) were dissolved in 168 mL of DMF, and bromopropylene d5-2 (4.4 g, 36.66 mmol) was added dropwise. The mixture was heated to 40° C. to react for 5 hours, and then the reaction was stopped. 500 mL of water was added to the reaction solution. The mixture was extracted with ethyl acetate, dried over anhydrous sodium sulfate, and concentrated. The crude was separated by column chromatography (PE/EA, 20/1) to obtain d5-3 (8.4 g) as a yellow solid, with a yield of 87%. LCMS ESI-MS m/z: 314 [M+H]⁺.

Step 2: Under nitrogen, the intermediate d5-3 (8.4 g, 26.66 mmol) from the previous step and potassium carbonate (7.3 g, 53.3 mmol) were dissolved in 84 mL of DMF. Catalyst Pd(OAc)₂ (0.6 g, 2.66 mmol) and tetrabutylammonium iodide TBAI (4.9 g, 13.3 mmol) were added. The mixture was heated to 80° C. to react for 4 hours and the reaction was stopped. 160 mL of water was added to the reaction solution. The mixture was extracted with ethyl acetate, dried over anhydrous sodium sulfate, and concentrated. The crude was separated by flash reverse column chromatography (Column: C18; CH₃CN/H₂O, 1/1) to give d5-4 (2.0 g) as a yellow oil, with a yield of 32%. LCMS ESI-MS m/z: 234 [M+H]⁺.

Step 3: Under nitrogen, the intermediate d5-4 (2.0 g, 8.54 mmol) from the previous step and the raw material benzophenonimine (3.1 g, 17.1 mmol) were dissolved in 20 mL of toluene. Catalyst Pd(OAc)₂ (200 mg, 1.06 mmol), the ligand BINAP (1.1 g, 1.7 mmol) and cesium carbonate (8.35 g, 25.6 mmol) were added. The mixture was heated to 110° C. to react for 1 hour. The reaction was stopped, and the mixture was filtered. 100 mL of water was added to the reaction solution. The mixture was extracted with ethyl acetate, dried over anhydrous sodium sulfate, and concentrated to obtain a crude mixture d5-5. LCMS ESI-MS m/z: 335 [M+H]⁺.

Step 4: The mixture d5-5 from the previous step was dissolved in 14 mL tetrahydrofuran, and 2M dilute hydrochloric acid solution (20 mL) was added. The mixture was reacted at room temperature for 1 hour, and the reaction was stopped. Saturated aqueous sodium bicarbonate solution was added to the reaction solution to adjust the pH to about 8. The mixture was extracted with ethyl acetate, dried over anhydrous sodium sulfate, and concentrated to obtain a brown oily crude product. Saturated aqueous sodium bicarbonate solution was added to the reaction solution to adjust the pH to about 8. The mixture was extracted with ethyl acetate, dried over anhydrous sodium sulfate, and concentrated. The crude product was separated by flash reverse column chromatography (Column: C18; CH₃CN/H₂O, 1/1) to give d5-6 (500 mg) as a yellow solid, with a yield of 33%. LCMS ESI-MS m/z: 171 [M+H]⁺.

Step 5: Under hydrogen (4 atm), the intermediate d5-6 (350 mg, 2.06 mmol) from the previous step was dissolved in 7 mL of ethyl acetate and Pd/C (70 mg, 20% wt) was added. The mixture was reacted at room temperature for 6 hours. The reaction was stopped and the mixture was filtered. 20 mL of ammonia water was added to the reaction solution. The mixture was extracted with dichloromethane, dried over anhydrous sodium sulfate, and concentrated. The crude was separated by flash reverse column chromatography (Column: C18; CH₃CN/H₂O, 5/3) to give d5 (180 mg) as a light yellow solid, with a yield of 51%. LCMS ESI-MS m/z: 173 [M+H]⁺.

### Preparation of Intermediate d6

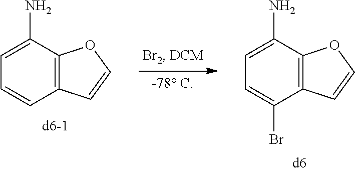 [SMILES: Nc1cccc2ccoc12 ; Nc1ccc(Br)c2ccoc12]

Procedure: At −78° C., under nitrogen, the raw material 7-aminobenzofuran d6-1 (600 mg, 4.13 mmol) was dissolved in 14 mL of anhydrous dichloromethane, and the pre-prepared liquid Br₂ solution (0.7 g, 4.13 mmol, DCM 11 mL) was added. The mixture was reacted at −78° C. for 1 hour and the reaction was stopped. 20 mL of saturated aqueous sodium thiosulfate solution was added to the reaction solution. The mixture was extracted with dichloromethane, dried over anhydrous sodium sulfate, and concentrated. The crude was separated by flash reverse column chromatography (Column: C18; CH₃CN/H₂O, 1/1) to give d6 (200 mg) as a yellow oil, with a yield of 23%. LCMS ESI-MS m/z: 212 [M+H]⁺.

### Example 2

### Preparation of Target Molecules P1-P3

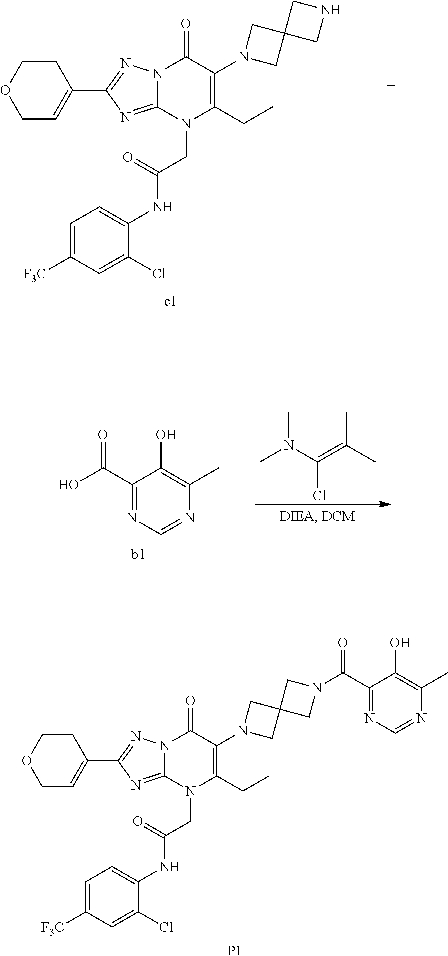

Procedure: Under nitrogen, intermediate b1 (16 mg, 0.1 mmol) and 1-chloro-N,N,2-trimethylpropyl-1-en-1-amine (13.9 mg, 0.1 mmol) were dissolved in 1 mL of dichloromethane and the mixture was stirred at room temperature for 1 hour. Intermediate ci (58 mg, 0.1 mmol) and DIEA (56 mg, 0.4 mmol) were added to the reaction solution, and the mixture was reacted at room temperature for another 1 hour, and the reaction was stopped. 10 mL of water was added to the reaction solution. The mixture was extracted with dichloromethane, dried over anhydrous sodium sulfate, and concentrated. The crude was separated by HPLC chromatography (Column: XSelect Prep OBD C18 Column, 30*150 mm, 5 pm; mobile phase A: water (10 mmol/L NH₄HCO₃), mobile phase B: acetonitrile; flow rate: 60 mL/min; retention time: 3 min) to give P1 (7.3 mg) as a white solid, with a yield of 11%. LCMS ESI-MS m/z: 714 [M+H]⁺.

Referring to the synthetic route of compound P1, the following target molecules were synthesized using similar raw materials/intermediates.

| | | | LC-MS |
|---|---|---|---|
| Target | | | ESI-MS |
| molecule | Molecular structure | ¹H NMR | m/z: [M + H]⁺ |
| P2 | 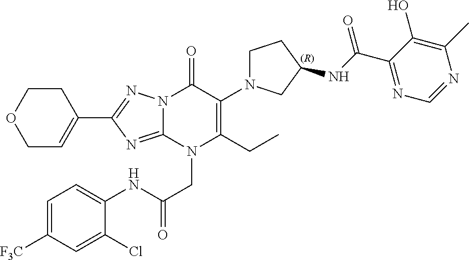 | ¹H NMR (300 MHz, DMSO-d₆) δ 12.43 (s, 1H), 10.40 (s, 1H), 9.57 (s, 1H), 8.69 (s, 1H), 8.08 (d, J = 8.5 Hz, 1H), 7.97 (s, 1H), 7.72 (d, J = 9.2 Hz, 1H), 6.85 (s, 1H), 5.34 (s, 2H), 4.68 (s, 1H), 4.27 (s, 2H), 3.82 (s, 2H), 3.49 (s, 1H), 3.17 (s, 3H), 2.97 (d, J = 8.0 Hz, 2H), 2.54 (s, 2H), 2.46 (s, 3H), 2.33-2.06 (m, 2H), 1.21 (t, J = 7.4 Hz, 3H). | 703 |
| P3 | 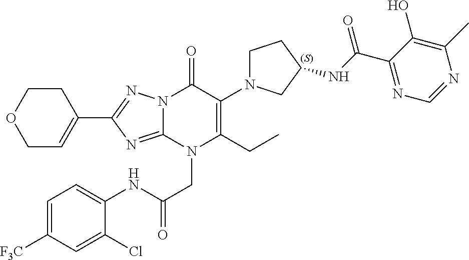 | ¹H NMR (300 MHz, DMSO-d₆) δ 12.42 (s, 1H), 10.40 (s, 1H), 9.68 (s, 1H), 8.65 (s, 1H), 8.08 (d, J = 8.6 Hz, 1H), 7.97 (d, J = 1.9 Hz, 1H), 7.73 (d, J = 8.8 Hz, 1H), 6.85 (s, 1H), 5.34 (s, 2H), 4.68 (s, 1H), 4.27 (s, 2H), 3.82 (t, J = 5.5 Hz, 2H), 3.54- 3.44 (m, 1H), 3.17 (s, 3H), 2.98 (d, J = 8.0 Hz, 2H), 2.54 (s, 2H), 2.45 (s, 3H), 2.30 (d, J = 12.6 Hz, 1H), 2.11 (d, J = 14.8 Hz, 1H), 1.21 (t, J = 7.6 Hz, 3H). | 703 |

### Example 3

### Preparation of Target Molecules H16-H18

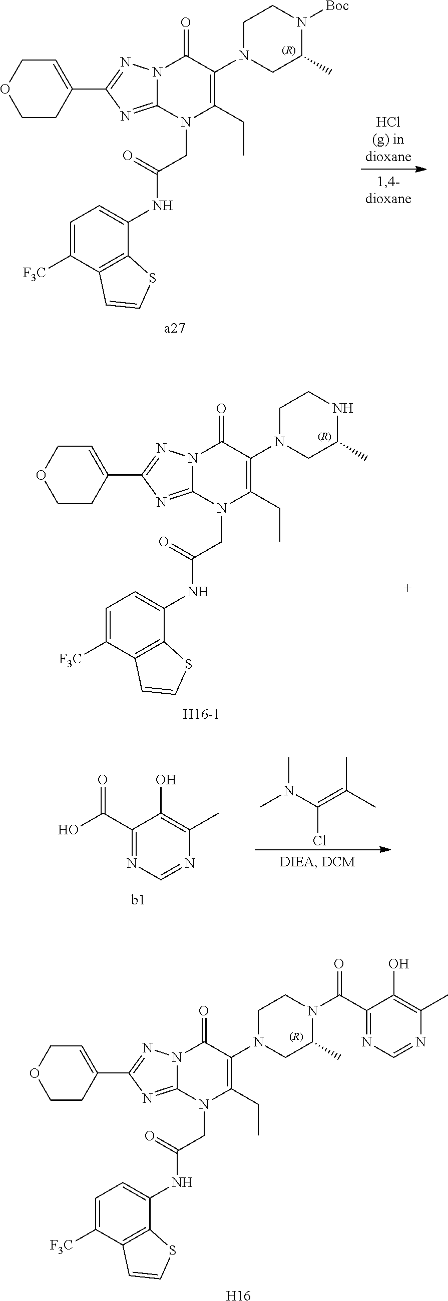

Step 1: Under nitrogen, intermediate a27 (160 mg, 0.2 mmol) was dissolved in 2 mL of HCl in 1,4-dioxane (2M), and the solution was stirred at room temperature for 1 hour. The solvent was removed by evaporation under reduced pressure to obtain H16-1 (105 mg) as a white solid, with a yield of 77%. LCMS ESI-MS m/z: 602 [M+H]⁺.

Step 2: Under nitrogen, intermediate b1 (70 mg, 0.4 mmol) and 1-chloro-N,N,2-trimethylpropyl-1-en-1-amine (81 mg, 0.5 mmol) were dissolved in 2 mL of dichloromethane and the mixture was stirred at room temperature for 1 hour. The compound H16-1 (105 mg, 0.175 mmol) from the previous step and DIEA (113 mg, 0.88 mmol) were added to the reaction solution. The mixture was reacted at room temperature for another 1 hour, and the reaction was stopped. 10 mL of water was added to the reaction solution. The mixture was extracted with dichloromethane, dried over anhydrous sodium sulfate, and concentrated. The crude was separated by HPLC chromatography (Column: XSelect Prep OBD C18 Column, 30*150 mm, 5 μm; mobile phase A: water (10 mmol/LNH₄HCO₃), mobile phase B: acetonitrile; flow rate: 60 mL/min; retention time: 7.36 min) to obtain H16 (6.4 mg) as a white solid, with a yield of 5%. LCMS ESI-MS m/z: 738 [M+H]⁺.

¹H NMR (400 MHz, DMSO-d₆) δ 10.88 (s, 1H), 8.44-7.98 (m, 2H), 7.80 (s, 2H), 7.57 (dd, J=5.6, 2.1 Hz, 1H), 6.85 (s, 1H), 5.35 (s, 2H), 4.86-4.19 (m, 4H), 3.80 (q, J=6.0, 5.6 Hz, 2H), 3.75-3.58 (m, 3H), 3.23-3.13 (m, 3H), 2.90 (d, J=54.8 Hz, 4H), 2.35 (d, J=32.0 Hz, 3H), 1.58 (s, 1H), 1.51-1.35 (m, 2H), 1.23 (dt, J=13.9, 8.1 Hz, 3H).

Referring to the synthetic route of compound H16, the following target molecules were synthesized using similar raw materials/intermediates (e.g. a27 or a30).

| | | | LC-MS |
|---|---|---|---|
| Target | | | ESI-MS |
| molecule | Molecular structure | ¹H NMR | m/z: [M + H]⁺ |
| H17 | 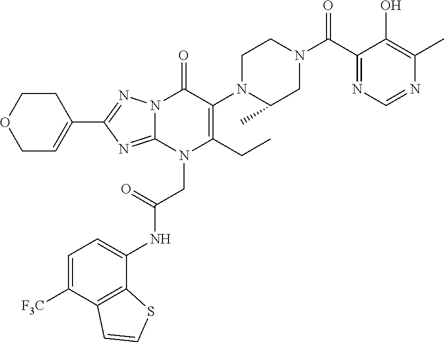 [SMILES: CCc1c(N2CCN(C(=O)c3ncnc(C)c3O)C[C@@H]2C)c(=O)n2nc(C3=CCOCC3)nc2n1CC(=O)Nc1ccc(C(F)(F)F)c2ccsc12] | ¹H NMR (300 MHz, DMSO-d₆) δ 8.53 (d, J = 4.5 Hz, 1H), 8.11 (d, J = 5.6 Hz, 1H), 7.80 (s, 2H), 7.57 (dd, J = 5.5, 2.3 Hz, 1H), 6.76 (d, J = 64.2 Hz, 1H), 5.56-5.17 (m, 2H), 4.51 (s, 1H), 4.41-4.18 (m, 2H), 3.82 (dt, J = 10.5, 5.2 Hz, 2H), 3.63 (dt, J = 10.5, 5.2 Hz, 1H), 3.38-3.05 (m, 4H), 3.08- 2.72 (m, 4H), 2.68 (d, J = 3.6 Hz, 1H), 2.44 (d, J = 3.6 Hz, 3H), 1.21 (q, J = 9.5, 8.3 Hz, 3H), 0.96-0.61 (m, 3H). | 738 |
| H18 | 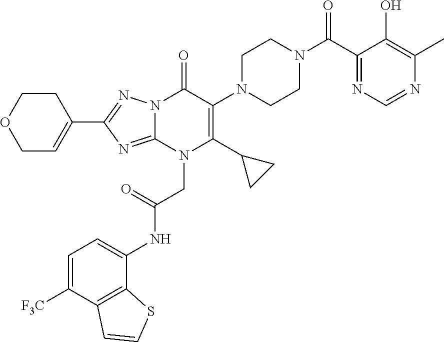 [SMILES: Cc1ncnc(C(=O)N2CCN(c3c(C4CC4)n(CC(=O)Nc4ccc(C(F)(F)F)c5ccsc45)c4nc(C5=CCOCC5)nn4c3=O)CC2)c1O] | ¹H NMR (400 MHz, DMSO-d₆) δ 8.54 (s, 1H), 8.12 (d, J = 5.6 Hz, 1H), 7.82 (d, J = 8.2 Hz, 1H), 7.73 (d, J = 8.1 Hz, 1H), 7.57 (dt, J = 5.5, 2.0 Hz, 1H), 6.63 (s, 1H), 5.13 (s, 2H), 4.30 (q, J = 2.8 Hz, 2H), 3.83 (t, J = 5.4 Hz, 2H), 3.51 (s, 5H), 2.86 (d, J = 30.5 Hz, 4H), 2.43 (s, 3H), 1.03-0.91 (m, 4H). | 736 |

### Example 4

### Preparation of Target Molecules P4-P6, H8, and H12

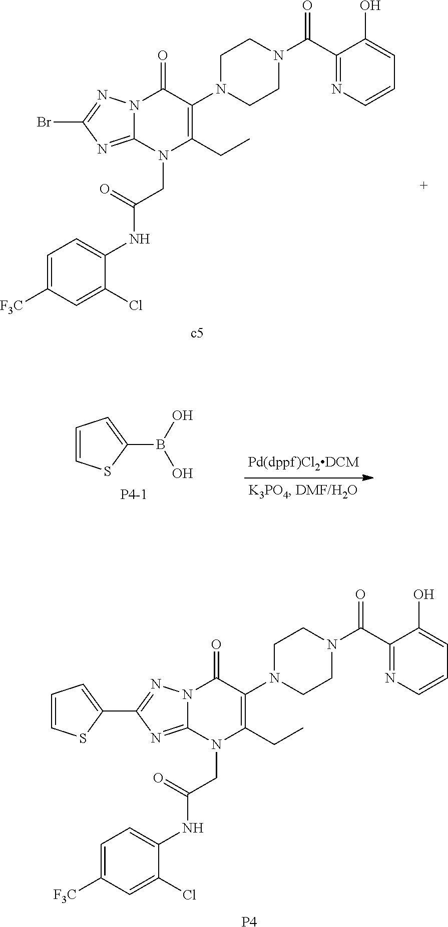

Procedure: Under nitrogen, intermediate c5 (55 mg, 0.08 mmol), raw material thiophene 2-carboxylic acid P4-1 (11 mg, 0.08 mmol) and potassium phosphate (51 mg, 0.24 mmol) were dissolved in 1 mL of a mixed solution of DMF and water (v/v, 4/1). Catalyst Pd(dppf)Cl₂·CH₂Cl₂ (6.6 mg, 0.01 mmol) was added. The mixture was heated to 90° C. to react for 1 hour, and the reaction was stopped. 5 mL of water was added to the reaction solution. The mixture was extracted with ethyl acetate, dried over anhydrous sodium sulfate, and concentrated. The crude product was separated by HPLC chromatography (Column: Xbridge Prep phenyl OBD Colum, 30*150 nm, 5 M; mobile phase A: water (10 mmol/L NH₄HCO₃), mobile phase B: acetonitrile; flow rate: 60 mL/min, retention time: 8.7 min;) to obtain P4 (10.1 mg) as a white solid, with a yield of 18%. LCMS ES-MS m/z: 687 [M+H].

¹H NMR (300 MHz, DMSO-d₆) δ 10.42 (s, 2H), 8.06 (d, J=8.2 Hz, 2H), 7.97 (d, J=2.0 Hz, 1H), 7.74 (dd, J=13.0, 6.5 Hz, 3H), 7.30 (s, 2H), 7.21 (t, J=4.3 Hz, 1H), 5.37 (s, 2H), 4.57 (d, J=12.1 Hz, 1H), 3.48 (s, 3H), 3.42 (d, J=13.3 Hz, 1H), 3.26 (d, J=13.2 Hz, 3H), 2.99 (d, J=12.7 Hz, 1H), 2.82 (d, J=11.3 Hz, 1H), 2.62 (d, J=28.8 Hz, 1H), 1.21 (t, J=7.7 Hz, 4H).

Referring to the synthetic route of compound P4, the following target molecules were synthesized using similar raw materials/intermediates (e.g. c7 or c8).

| | | | LC-MS |
|---|---|---|---|
| Target | | | ESI-MS |
| molecule | Molecular structure | ¹H NMR | m/z: [M + H]⁺ |
| P5 | 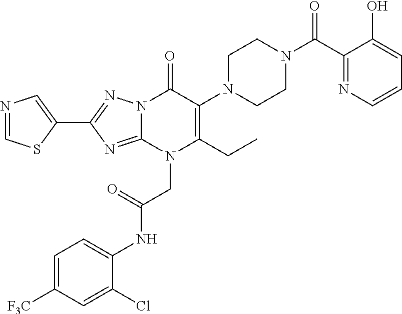 [SMILES: CCc1c(N2CCN(C(=O)c3ncccc3O)CC2)c(=O)n2nc(-c3cncs3)nc2n1CC(=O)Nc1ccc(C(F)(F)F)cc1Cl] | ¹H NMR (400 MHz, DMSO-d₆) δ 9.27 (s, 1H), 8.50 (s, 1H), 8.36 (s, 1H), 8.05-7.98 (d, J = 8.7 Hz, 3H), 7.72 (dd, J = 8.8, 2.2 Hz, 1H), 7.28 (dd, J = 12.5, 7.8 Hz, 2H), 5.38 (s, 2H), 4.56 (d, J = 12.5 Hz, 1H), 3.52-2.91 (m, 8H), 2.82 (d, J = 11.2 Hz, 1H), 2.65 (d, J = 10.7 Hz, 1H), 1.25-1.16 (m, 3H). | 689 |
| P6 | 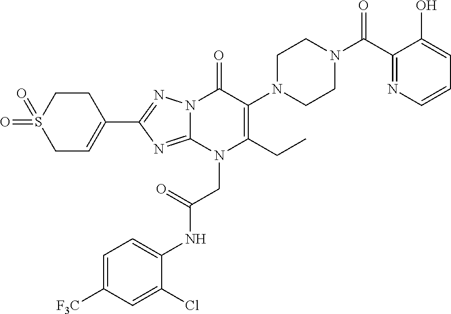 [SMILES: CCc1c(N2CCN(C(=O)c3ncccc3O)CC2)c(=O)n2nc(C3=CCS(=O)(=O)CC3)nc2n1CC(=O)Nc1ccc(C(F)(F)F)cc1Cl] | ¹H NMR (300 MHz, DMSO-d₆) δ 10.38 (s, 2H), 8.10-7.96 (m, 3H), 7.71 (d, J = 8.4 Hz, 1H), 7.29 (d, J = 2.6 Hz, 2H), 6.76 (s, 1H), 5.32 (s, 2H), 4.55 (d, J = 12.3 Hz, 1H), 3.99 (s, 2H), 3.51 (s, 2H), 3.37 (s, 3H), 3.22 (s, 1H), 3.10 (s, 2H), 2.99 (s, 3H), 2.80 (d, J = 10.7 Hz, 1H), 2.63 (d, J = 10.5 Hz, 1H), 1.20 (q, J = 7.4 Hz, 4H). | 735 |
| H8 | 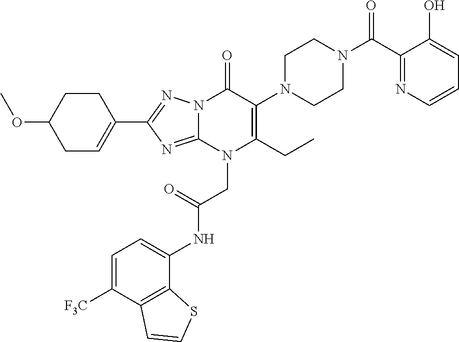 [SMILES: CCc1c(N2CCN(C(=O)c3ncccc3O)CC2)c(=O)n2nc(C3=CCC(OC)CC3)nc2n1CC(=O)Nc1ccc(C(F)(F)F)c2ccsc12] | ¹H NMR (300 MHz, DMSO-d₆) δ 10.78 (s, 1H), 10.46 (s, 1H), 8.12 (d, J = 5.6 Hz, 1H), 8.07 (dd, J = 3.5, 2.4 Hz, 1H), 7.87-7.77 (m, 2H), 7.57 (dq, J = 5.6, 1.9 Hz, 1H), 7.33-7.26 (m, 2H), 6.76 (d, J = 4.1 Hz, 1H), 5.33 (s, 2H), 4.56 (d, J = 12.4 Hz, 1H), 3.54-3.47 (m, 2H), 3.44 (d, J = 9.3 Hz, 1H), 3.39 (s, 1H), 3.26 (s, 3H), 3.24-3.18 (m, 1H), 3.00 (ddd, J = 28.0, 12.9, 8.0 Hz, 3H), 2.81 (d, J = 11.2 Hz, 1H), 2.63 (d, J = 11.4 Hz, 2H), 2.56 (d, J = 6.9 Hz, 1H), 2.44 (d, J = 17.0 Hz, 1H), 2.15 (ddd, J = 18.4, 6.5, 3.3 Hz, 1H), 2.00-1.89 (m, 1H), 1.68 (ddd, J = 13.2, 9.0, 5.7 Hz, 1H), 1.22 (t, J = 7.5 Hz, 3H). | 737 |
| H12 | 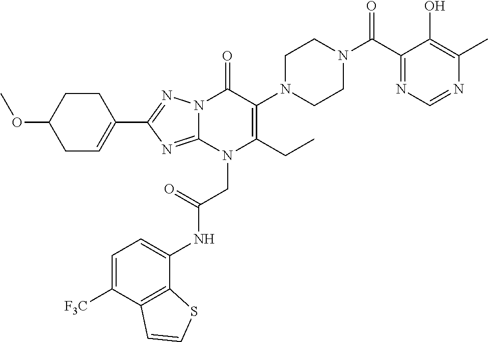 | ¹H NMR (300 MHz, DMSO-d₆) δ 8.52 (s, 1H), 8.12 (d, J = 5.6 Hz, 1H), 7.86-7.74 (m, 2H), 7.57 (dt, J = 5.6, 2.0 Hz, 1H), 6.76 (s, 1H), 5.33 (s, 2H), 4.52 (d, J = 12.5 Hz, 1H), 3.50 (s, 5H), 3.38 (s, 3H), 3.03 (d, J = 7.9 Hz, 3H), 2.82 (d, J = 11.1 Hz, 1H), 2.64 (d, J = 10.8 Hz, 4H), 2.56 (d, J = 4.2 Hz, 3H), 2.11 (s, 1H), 1.94 (s, 1H), 1.65 (s, 1H), 1.27 (m, 3H). | 752 |

### Example 5

### Preparation of Target Molecules H1-H7, H13-H15, H19-H26, and A2

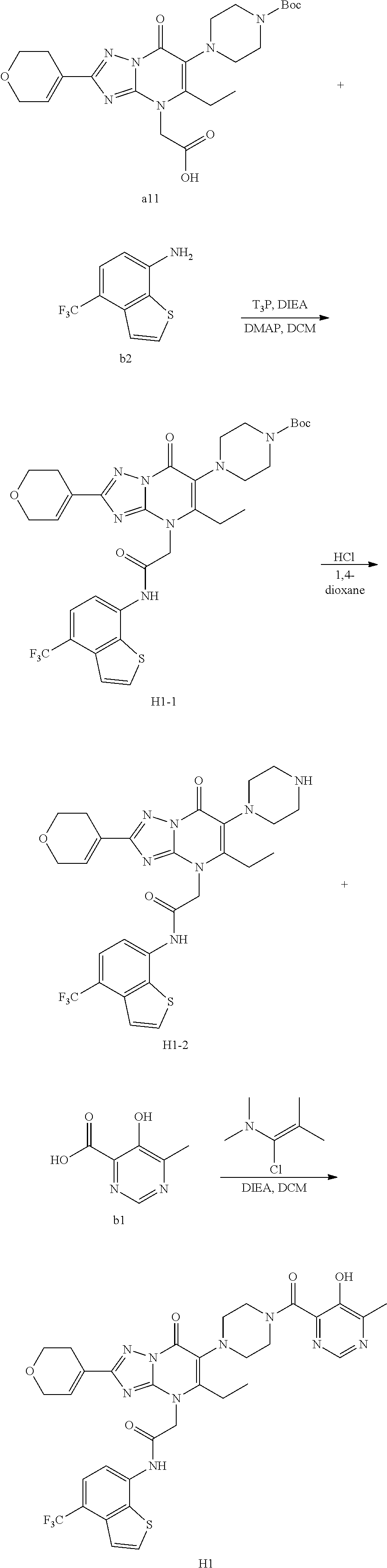

Step 1: Under nitrogen, intermediate b2 (50 mg, 0.23 mmol), DIEA (148 mg, 1.15 mmol) and intermediate all (90 mg, 0.18 mmol) were dissolved in 1 mL of dichloromethane and the mixture was stirred at room temperature for 5 minutes. T₃P (146 mg, 0.46 mmol) and DMAP (11 mg, 0.09 mmol) were added. The mixture was reacted at room temperature for another 3 hours and the reaction was stopped. 10 mL of water was added to the reaction solution. The mixture was extracted with dichloromethane, dried over anhydrous sodium sulfate, and concentrated. The crude was separated by column chromatography (PE/EA, 50/1) to obtain Hi-1 (60 mg) as a yellow solid, with a yield of 38%. LCMS ESI-MS m/z: 688 [M+H]⁺.

Step 2: Compound Hi-1 (60 mg, 0.08 mmol) from the previous step was dissolved in 1 mL 1,4-dioxane, and HCl/1,4-dioxane solution (1M, 1.2 mL) was added. The mixture was reacted at room temperature for 1 hour, and then the reaction was stopped. 10 mL of water was added to the reaction solution, and the pH was adjusted to about 9 with saturated sodium bicarbonate. The mixture was extracted with dichloromethane, dried over anhydrous sodium sulfate, and concentrated to obtain Hi-2 (40 mg) as a yellow solid, with a yield of 78%. LCMS ESI-MS m/z: 588 [M+H]⁺.

Step 3: In an ice bath, under nitrogen, intermediate bi (32 mg, 0.2 mmol) and 1-chloro-N,N,2-trimethylpropyl-1-en-1-amine (27 mg, 0.2 mmol) were dissolved in 2 mL of dichloromethane and the mixture was stirred at room temperature for 1 hour. Compound H1-2 (60 mg, 0.1 mmol) and DIEA (66 mg, 0.5 mmol) were added to the reaction solution. The mixture was reacted at room temperature for 4 hours, and then the reaction was stopped. 10 mL of water was added to the reaction solution. The mixture was extracted with dichloromethane, dried over anhydrous sodium sulfate, and concentrated. The crude was separated by HPLC chromatography (Column: Column: Xselect CSH Prep Fluoro-phenyl Column, 30*150 nm, 5 m; mobile phase A: water (0.1% formic acid), mobile phase B: acetonitrile; flow rate: 60 mL/min; retention time: 9.4 mi) to obtain H1 (7.3 mg) as a white solid, with a yield of 21%. LCMS ES-MS m/z: 724 [M+H]⁺.

¹H NMR (300 MHz, DMSO-d₆) δ 10.85 (s, 1H), 8.57 (s, 1H), 8.12 (d, J=5.6 Hz, 1H), 7.84-7.76 (m, 2H), 7.62-7.51 (m, 1H), 6.86 (s, 1H), 5.35 (s, 2H), 4.53 (d, J=12.5 Hz, 1H), 4.27-4.23 (m, 2H), 3.81 (t, J=5.5 Hz, 2H), 3.51 (d, J=12.0 Hz, 4H), 3.26 (t, J=12.3 Hz, 2H), 3.02 (d, J=11.4 Hz, 3H), 2.83 (d, J=11.3 Hz, 1H), 2.66 (d, J=11.3 Hz, 1H), 2.45 (s, 3H), 1.22 (t, J=7.3 Hz, 3H).

Referring to the synthetic route of compound HI, the following target molecules were synthesized using similar raw materials/intermediates (e.g. b3-b6 or d1-d6).

| | | | LC-MS |
|---|---|---|---|
| Target | | | ESI-MS |
| molecule | Molecular structure | ¹H NMR | m/z: [M + H]⁺ |
| H2 | 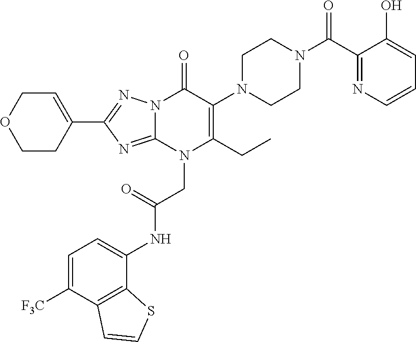 [SMILES: CCc1c(N2CCN(C(=O)c3ncccc3O)CC2)c(=O)n2nc(C3=CCOCC3)nc2n1CC(=O)Nc1ccc(C(F)(F)F)c2ccsc12] | ¹H NMR (300 MHz, DMSO-d₆) δ 10.68 (s, 2H), 8.11-8.02 (m, 2H), 7.86-7.72 (m, 2H), 7.58-7.51 (m, 1H), 7.32-7.25 (m, 2H), 6.85 (s, 1H), 5.30 (s, 2H), 4.56 (d, J = 12.4 Hz, 1H), 4.25 (d, J = 2.9 Hz, 2H), 3.85-3.74 (m, 2H), 3.46 (d, J = 12.2 Hz, 4H), 3.23 (s, 2H), 2.97 (d, J = 26.4 Hz, 3H), 2.82 (d, J = 11.2 Hz, 1H), 2.64 (d, J = 10.9 Hz, 1H), 1.22 (t, J = 7.0 Hz, 3H). | 709 |
| H3 | 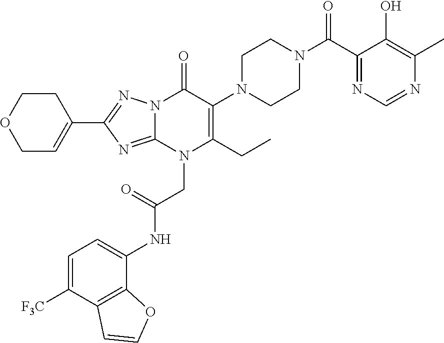 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ccoc12] | ¹H NMR (400 MHz, DMSO-d₆) δ 10.37 (s, 1H), 8.55 (s, 1H), 7.93 (d, J = 8.5 Hz, 1H), 7.12 (d, J = 8.6 Hz, 1H), 6.85-6.79 (m, 1H), 5.27 (s, 2H), 4.76 (t, J = 8.8 Hz, 2H), 4.52 (d, J = 12.5 Hz, 1H), 4.25 (q, J = 2.8 Hz, 2H), 3.80 (t, J = 5.4 Hz, 2H), 3.59- 3.45 (m, 4H), 3.41 (t, J = 8.7 Hz, 3H), 3.24 (t, J = 12.3 Hz, 2H), 2.97 (dd, J = 20.6, 9.9 Hz, 3H), 2.81 (d, J = 11.2 Hz, 1H), 2.69-2.61 (m, 1H), 2.44 (s, 3H), 1.16 (t, J = 7.4 Hz, 3H). | 710 |
| H4 | 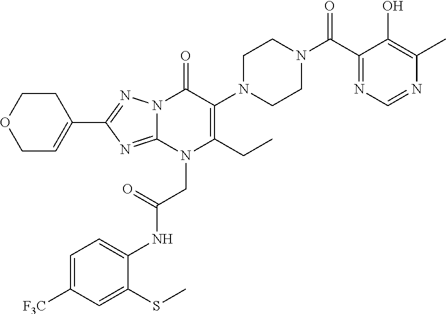 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1SC] | ¹H NMR (300 MHz, DMSO-d₆) δ 10.28 (s, 1H), 10.16 (s, 1H), 8.57 (s, 1H), 7.70-7.57 (m, 2H), 7.53 (dd, J = 8.9, 2.0 Hz, 1H), 6.91-6.84 (m, 1H), 5.24 (s, 2H), 4.53 (d, J = 12.4 Hz, 1H), 4.27 (q, J = 2.8 Hz, 2H), 3.81 (t, J = 5.4 Hz, 2H), 3.49 (t, J = 10.8 Hz, 3H), 3.25 (t, J = 12.3 Hz, 3H), 3.09-2.90 (m, 3H), 2.81 (d, J = 11.0 Hz, 1H), 2.64 (d, J = 10.8 Hz, 1H), 2.54 (s, 3H), 2.44 (s, 3H), 1.21 (t, J = 7.4 Hz, 3H). | 714 |
| H5 | 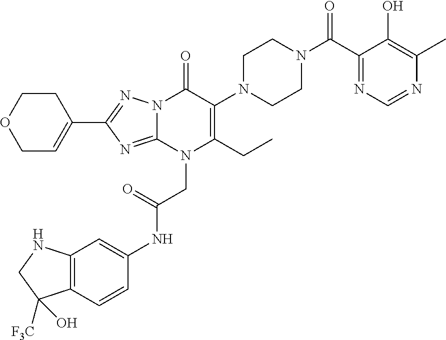 [SMILES: CCc1c(N2CCN(C(=O)c3ncnc(C)c3O)CC2)c(=O)n2nc(C3=CCOCC3)nc2n1CC(=O)Nc1ccc2c(c1)NCC2(O)C(F)(F)F] | ¹H NMR (400 MHz, DMSO-d₆) δ 10.43 (s, 1H), 8 7.94 (s, 1H), 7.09 (d, J = 8.1 Hz, 1H), 6.93 (s, 1H), 6.71 (d, J = 14.6 Hz, 2H), 6.59 (s, 1H), 5.93 (s, 1H), 5.09 (s, 1H), 4.60 (s, 1H), 4.27 (s, 2H), 3.74-3.59 (m, 3H), 3.28 (m, 9H), 2.98 (s, 3H), 2.77 (s, 1H), 2.65 (s, 1H), 2.28 (s, 3H), 1.34 (d, J = 13.6 Hz, 3H). | 725 |
| H6 | 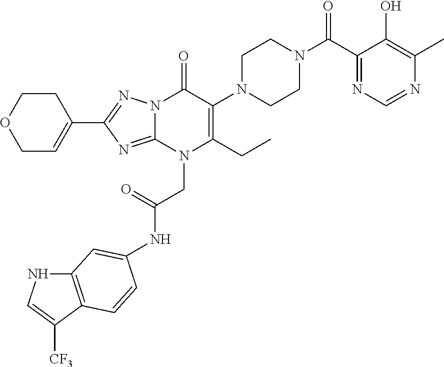 [SMILES: CCc1c(N2CCN(C(=O)c3ncnc(C)c3O)CC2)c(=O)n2nc(C3=CCOCC3)nc2n1CC(=O)Nc1ccc2c(C(F)(F)F)c[nH]c2c1] | ¹H NMR (300 MHz, DMSO-d₆) δ 11.8 (s, 1H), 10.61 (s, 1H), 8.45 (s, 1H), 8.02 (d, J = 1.8 Hz, 1H), 7.89 (d, J = 1.6 Hz, 1H), 7.54 (d, J = 8.6 Hz, 1H), 7.22 (dd, J = 8.7, 1.7 Hz, 1H), 6.81 (s, 1H), 5.19 (s, 2H), 4.52 (d, J = 12.7 Hz, 1H), 4.27-4.20 (m, 2H), 3.79 (s, 2H), 3.49 (s, 7H), 2.98 (d, J = 9.4 Hz, 3H), 2.81 (d, J = 11.6 Hz, 1H), 2.64 (d, J = 10.9 Hz, 1H), 2.41 (s, 3H), 1.20 (q, J = 8.3, 7.4 Hz, 3H). | 707 |
| H7 | 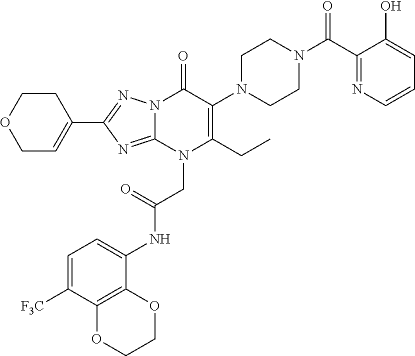 [SMILES: CCc1c(N2CCN(C(=O)c3ncccc3O)CC2)c(=O)n2nc(C3=CCOCC3)nc2n1CC(=O)Nc1ccc(C(F)(F)F)c2c1OCCO2] | ¹H NMR (300 MHz, DMSO-d₆) δ 10.42 (s, 1H), 10.15 (s, 1H), 8.06 (dd, J = 3.6, 2.3 Hz, 1H), 7.71 (d, J = 8.7 Hz, 1H), 7.38-7.23 (m, 2H), 7.13 (d, J = 8.9 Hz, 1H), 6.81 (t, J = 1.7 Hz, 1H), 5.31 (s, 2H), 4.55 (d, J = 12.4 Hz, 1H), 4.44 (d, J = 2.6 Hz, 4H), 4.25 (q, J = 2.8 Hz, 2H), 3.80 (t, J = 5.5 Hz, 2H), 3.56-3.37 (m, 4H), 3.22 (s, 2H), 2.97 (d, J = 13.1 Hz, 3H), 2.80 (d, J = 11.6 Hz, 1H), 2.62 (d, J = 10.9 Hz, 1H), 1.16 (t, J = 7.4 Hz, 3H). | 711 |
| H13 | 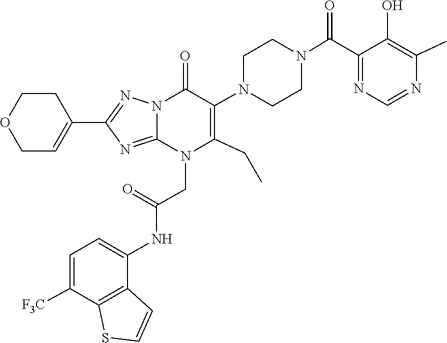 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2sccc12] | ¹H NMR (300 MHz, DMSO-d₆) 10.76 (s, 1H), 8.52 (s, 1H), 8.07- 8.00 (m, 2H), 7.96 (d, J = 5.7 Hz, 1H), 7.78 (d, J = 8.4 Hz, 1H), 6.82 (s, 1H), 5.37 (s, 2H), 4.52 (d, J = 12.5 Hz, 1H), 4.34-4.16 (m, 2H), 3.87-3.71 (m, 2H), 3.50 (d, J = 10.7 Hz, 4H), 3.24 (s, 3H), 3.12- 2.93 (m, 3H), 2.82 (d, J = 10.8 Hz, 1H), 2.65 (d, J = 10.4 Hz, 1H), 2.43 (s, 3H), 1.24-1.16 (m, 3H). | 724 |
| H14 | 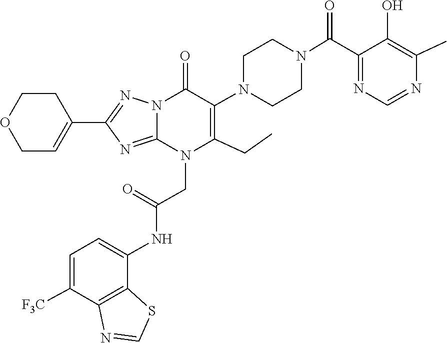 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ncsc12] | ¹H NMR (400 MHz, DMSO-d₆) δ 11.06 (s, 1H), 9.60 (s, 1H), 8.58 (s, 1H), 8.00-7.83 (m, 2H), 6.84 (s, 1H), 5.33 (s, 2H), 4.53 (d, J = 12.6 Hz, 1H), 4.25 (s, 2H), 3.80 (t, J = 5.5 Hz, 2H), 3.51 (d, J = 12.6 Hz, 3H), 3.26 (s, 1H), 3.11-2.92 (m, 4H), 2.84 (d, J = 11.3 Hz, 1H), 2.66 (d, J = 11.7 Hz, 2H), 2.45 (s, 3H), 1.21 (t, J = 7.5 Hz, 3H). | 725 |
| H15 | 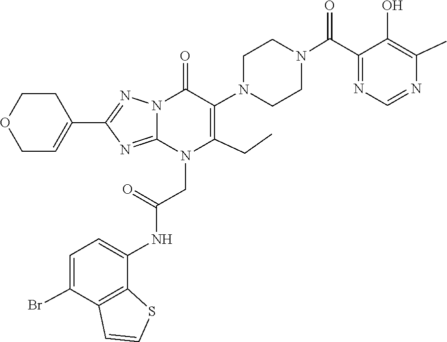 [SMILES: CCc1c(N2CCN(C(=O)c3ncnc(C)c3O)CC2)c(=O)n2nc(C3=CCOCC3)nc2n1CC(=O)Nc1ccc(Br)c2ccsc12] | ¹H NMR (300 MHz, DMSO-d₆) δ 10.66 (s, 1H), 8.50 (s, 1H), 7.97 (d, J = 5.5 Hz, 1H), 7.63 (d, J = 8.2 Hz, 1H), 7.53-7.42 (m, 2H), 6.87 (s, 1H), 5.29 (s, 2H), 4.52 (d, J = 12.5 Hz, 1H), 4.26 (s, 2H), 3.81 (s, 3H), 3.50 (s, 6H), 3.03 (s, 3H), 2.82 (d, J = 11.4 Hz, 1H), 2.66 (s, 1H), 2.43 (s, 3H), 1.24 (s, 3H). | 734 |
| H19 | 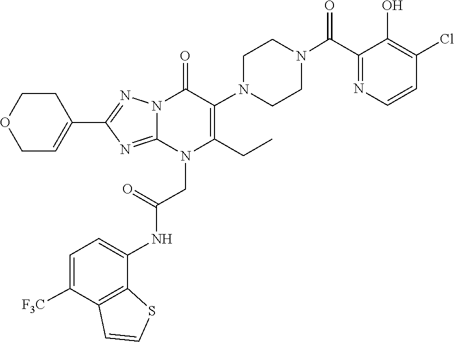 [SMILES: CCc1c(N2CCN(C(=O)c3nccc(Cl)c3O)CC2)c(=O)n2nc(C3=CCOCC3)nc2n1CC(=O)Nc1ccc(C(F)(F)F)c2ccsc12] | ¹H NMR (300 MHz, DMSO-d₆) δ 8.11 (d, J = 5.5 Hz, 1H), 7.80 (d, J = 1.6 Hz, 2H), 7.72 (s, 1H), 7.62-7.53 (m, 1H), 7.35 (s, 1H), 6.86 (s, 1H), 5.34 (s, 2H), 4.53 (d, J = 12.6 Hz, 1H), 4.25 (s, 2H), 3.80 (s, 2H), 3.49 (d, J = 14.3 Hz, 4H), 3.03 (d, J = 7.8 Hz, 3H), 2.78 (s, 1H), 2.62 (d, J = 10.2 Hz, 1H), 1.28-1.17 (m, 3H). | 743 |
| H20 | 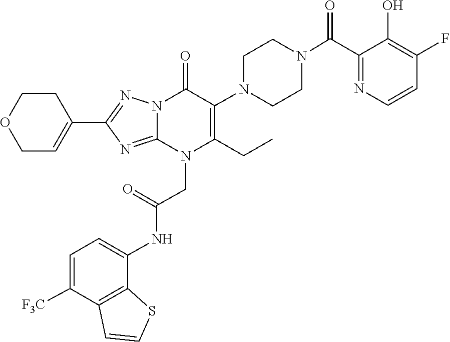 [SMILES: CCc1c(N2CCN(C(=O)c3nccc(F)c3O)CC2)c(=O)n2nc(C3=CCOCC3)nc2n1CC(=O)Nc1ccc(C(F)(F)F)c2ccsc12] | ¹H NMR (300 MHz, DMSO-d₆) δ 8.12 (d, J = 5.6 Hz, 1H), 8.03 (s, 1H), 7.80 (s, 2H), 7.57 (d, J = 5.1 Hz, 1H), 7.32 (d, J = 8.9 Hz, 1H), 6.86 (s, 1H), 5.35 (s, 2H), 4.55 (d, J = 12.3 Hz, 1H), 4.25 (s, 2H), 3.80 (s, 2H), 3.47 (s, 3H), 3.28 (s, 3H), 3.03 (s, 3H), 2.82 (d, J = 11.0 Hz, 1H), 2.64 (d, J = 10.8 Hz, 1H), 1.27-1.17 (m, 3H). | 727 |
| H21 | 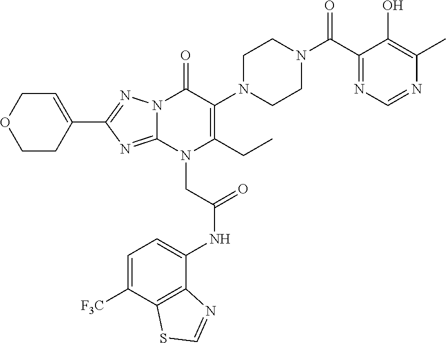 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2scnc12] | ¹H NMR (400 MHz, DMSO-d₆) δ 11.23 (s, 1H), 10.40 (s, 1H), 9.66 (s, 1H), 8.56 (s, 1H), 8.46 (d, J = 8.4 Hz, 1H), 7.91 (d, J = 8.5 Hz, 1H), 6.80 (p, J = 1.4 Hz, 1H), 5.49 (s, 2H), 4.53 (d, J = 12.4 Hz, 1H), 4.22 (q, J = 2.8 Hz, 2H), 3.79 (t, J = 5.5 Hz, 2H), 3.58-3.45 (m, 4H), 3.30- 3.20 (m, 2H), 2.98 (p, J = 7.0, 5.3 Hz, 3H), 2.83 (d, J = 11.2 Hz, 1H), 2.70-2.63 (m, 1H), 2.44 (s, 3H), 1.18 (t, J = 7.4 Hz, 3H). | 725 |
| H22 | 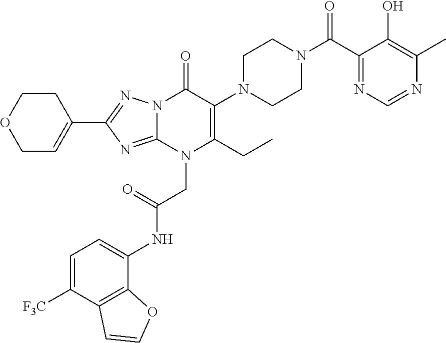 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ccoc12] | ¹H NMR (400 MHz, DMSO-d₆) δ 11.12 (s, 1H), 8.52 (s, 1H), 8.35 (d, J = 2.2 Hz, 1H), 8.06 (d, J = 8.4 Hz, 1H), 7.60 (d, J = 8.5 Hz, 1H), 7.14 (s, 1H), 6.81 (s, 1H), 5.38 (s, 2H), 4.53 (d, J = 12.6 Hz, 1H), 4.26-4.21 (m, 2H), 3.79 (t, J = 5.6 Hz, 2H), 3.51-3.27 (s, 6H), 3.00 (d, J = 10.7 Hz, 3H), 2.82 (d, J = 11.4 Hz, 1H), 2.67 (s, 1H), 2.44 (s, 3H), 1.20 (t, J = 7.5 Hz, 3H). | 708 |
| H23 | 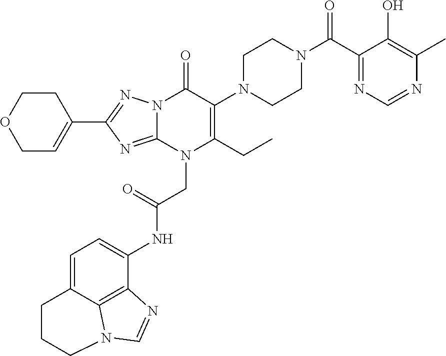 [SMILES: CCc1c(N2CCN(C(=O)c3ncnc(C)c3O)CC2)c(=O)n2nc(C3=CCOCC3)nc2n1CC(=O)Nc1ccc2c3c1ncn3CCC2] | ¹H NMR (400 MHz, DMSO-d₆) δ 10.62 (s, 1H), 8.57 (s, 1H), 8.22 (d, J = 10.4 Hz, 1H), 7.75 (d, J = 7.8 Hz, 1H), 6.91 (d, J = 7.9 Hz, 1H), 6.83 (s, 1H), 5.35 (s, 2H), 4.53 (d, J = 12.5 Hz, 1H), 4.34-4.13 (m, 4H), 3.80 (t, J = 5.5 Hz, 2H), 3.51 (d, J = 11.4 Hz, 4H), 3.32-3.19 (m, 2H), 3.11-2.87 (m, 5H), 2.83 (d, J = 11.2 Hz, 1H), 2.70-2.62 (m, 1H), 2.45 (s, 3H), 2.13 (q, J = 6.2, 5.8 Hz, 2H), 1.19 (t, J = 7.4 Hz, 3H). | 680 |
| H24 | 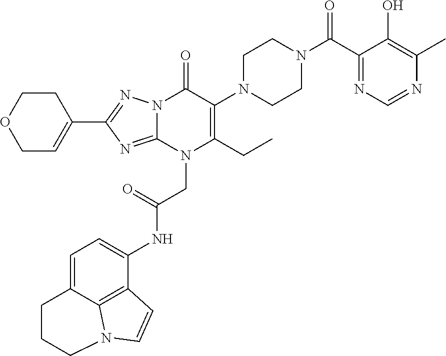 [SMILES: CCc1c(N2CCN(C(=O)c3ncnc(C)c3O)CC2)c(=O)n2nc(C3=CCOCC3)nc2n1CC(=O)Nc1ccc2c3c1ccn3CCC2] | ¹H NMR (300 MHz, DMSO-d₆) δ 10.20 (s, 1H), 8.56 (s, 1H), 7.39 (d, J = 7.6 Hz, 1H), 7.29 (d, J = 3.0 Hz, 1H), 6.82 (s, 1H), 6.75 (d, J = 7.8 Hz, 1H), 6.61 (d, J = 3.0 Hz, 1H), 5.28 (s, 2H), 4.53 (d, J = 12.7 Hz, 1H), 4.23 (s, 2H), 4.15 (s, 2H), 3.79 (d, J = 5.9 Hz, 2H), 3.51-3.54 (s, 1H), 3.27 (d, J = 12.9 Hz, 2H), 2.99 (s, 3H), 2.92-2.84 (m, 3H), 2.81 (s, 1H), 2.65 (d, J = 10.6 Hz, 1H), 2.44 (s, 3H), 2.10 (s, 2H), 1.32- 1.04 (m, 5H). | 679 |
| H25 | 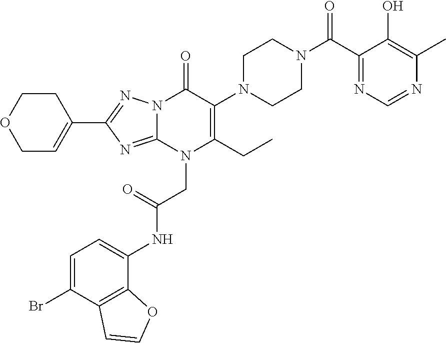 [SMILES: CCc1c(N2CCN(C(=O)c3ncnc(C)c3O)CC2)c(=O)n2nc(C3=CCOCC3)nc2n1CC(=O)Nc1ccc(Br)c2ccoc12] | ¹H NMR (300 MHz, DMSO-d₆) δ 10.82 (s, 1H), 10.32 (s, 1H), 8.56 (s, 1H), 8.24 (d, J = 2.3 Hz, 1H), 7.75 (d, J = 8.5 Hz, 1H), 7.43 (d, J = 8.4 Hz, 1H), 6.99 (d, J = 2.3 Hz, 1H), 6.82 (s, 1H), 5.31 (s, 2H), 4.53 (d, J = 12.6 Hz, 1H), 4.24 (t, J = 2.9 Hz, 2H), 3.80 (t, J = 5.5 Hz, 2H), 3.50 (q, J = 9.7, 6.6 Hz, 4H), 3.25 (t, J = 12.5 Hz, 2H), 2.98 (q, J = 8.3, 6.0 Hz, 3H), 2.82 (d, J = 11.5 Hz, 1H), 2.65 (d, J = 11.2 Hz, 1H), 2.44 (s, 3H), 1.20 (q, J = 8.6, 7.5 Hz, 3H). | 718 |
| H26 | 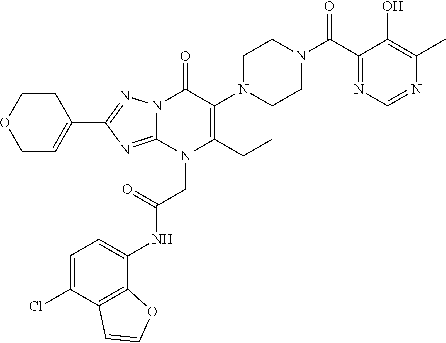 [SMILES: CCc1c(N2CCN(C(=O)c3ncnc(C)c3O)CC2)c(=O)n2nc(C3=CCOCC3)nc2n1CC(=O)Nc1ccc(Cl)c2ccoc12] | ¹H NMR (300 MHz, DMSO-d₆) δ 10.85 (s, 1H), 8.46 (s, 1H), 8.22 (d, J = 2.2 Hz, 1H), 7.79 (d, J = 8.6 Hz, 1H), 7.29 (d, J = 8.5 Hz, 1H), 7.07 (d, J = 2.2 Hz, 1H), 6.82 (s, 1H), 5.32 (s, 2H), 4.51 (d, J = 12.5 Hz, 1H), 4.24 (d, J = 3.5 Hz, 2H), 3.78 (d, J = 5.6 Hz, 2H), 3.50 (d, J = 8.7 Hz, 4H), 2.98 (d, J = 8.0 Hz, 3H), 2.81 (d, J = 10.8 Hz, 1H), 2.66 (s, 1H), 2.42 (s, 2H), 2.10 (d, J = 9.5 Hz, 1H), 1.21 (q, J = 7.8, 5.9 Hz, 4H). | 674 |
| A2 refer- ence mole- cule | 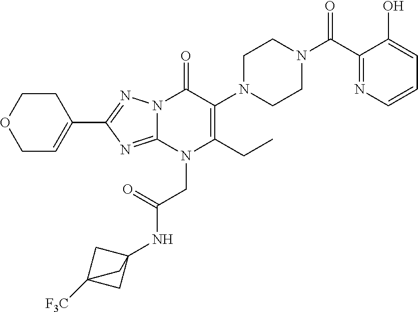 [SMILES: CCc1c(N2CCN(C(=O)c3ncccc3O)CC2)c(=O)n2nc(C3=CCOCC3)nc2n1CC(=O)NC12CC(C(F)(F)F)(C1)C2] | ¹H NMR (300 MHz, DMSO-d₆) δ 10.46 (s, 1H), 9.18 (s, 1H), 8.05 (t, J = 2.9 Hz, 1H), 7.28 (d, J = 3.0 Hz, 2H), 6.81 (dd, J = 3.3, 1.7 Hz, 1H), 4.90 (s, 2H), 4.54 (d, J = 12.5 Hz, 1H), 4.27 (q, J = 2.8 Hz, 2H), 3.82 (t, J = 5.4 Hz, 2H), 3.46 (t, J = 14.2 Hz, 4H), 3.23 (d, J = 12.1 Hz, 1H), 3.04-2.70 (m, 4H), 2.60 (d, J = 10.8 Hz, 1H), 2.24 (s, 6H), 1.14 (t, J = 7.4 Hz, 3H). | 643 |

### Example 6

### Preparation of Target Molecules H9-H11

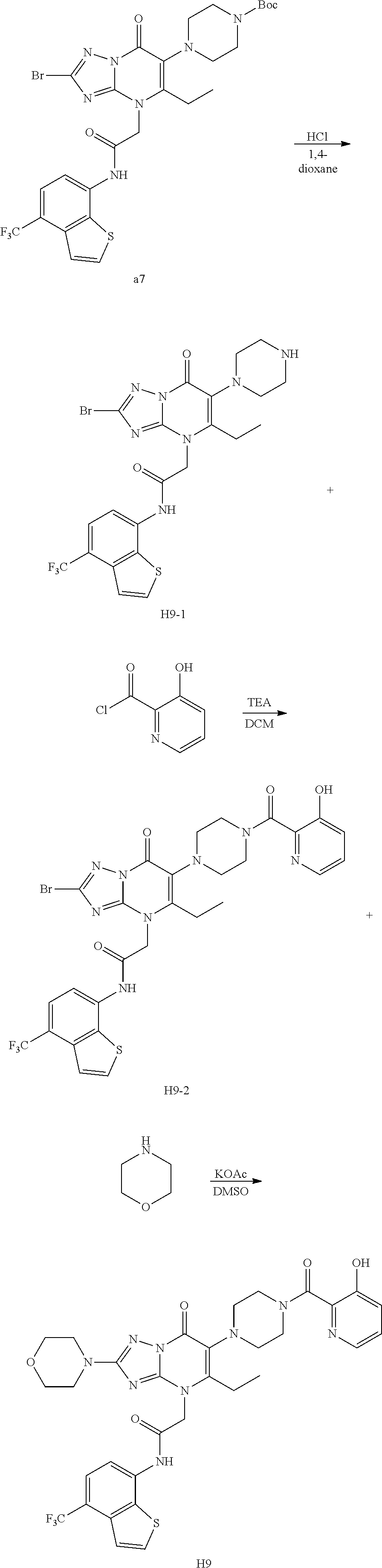

Step 1: Intermediate a7 (1.5 g, 2.19 mmol) was dissolved in 8 mL of 1,4-dioxane, and HCl/1,4-dioxane solution (1M, 30 mL) was added. The mixture was reacted at room temperature for 1 hour, and then the reaction was stopped. 10 mL of water was added to the reaction solution, and the pH was adjusted to about 9 with saturated sodium bicarbonate. The mixture was extracted with dichloromethane, dried over anhydrous sodium sulfate, and concentrated to obtain H9-1 (1.0 g) as a yellow solid, with a yield of 78%. LCMS ESI-MS m/z: 584 [M+H]⁺.

Step 2: In an ice bath, under nitrogen, the compound H9-1 (1.0 g, 1.71 mmol) from the previous step and TEA (520 mg, 5.13 mmol) were dissolved in 20 mL of dichloromethane, and compound 3-hydroxy-2-pyridinecarbonyl chloride (270 mg, 1.71 mmol) was added. The mixture was reacted at room temperature for 1 hour and the reaction was stopped. 50 mL of water was added to the reaction solution. The mixture was extracted with dichloromethane, dried over anhydrous sodium sulfate, and concentrated. The crude was separated by flash reverse column chromatography (C18, CH₃CN/H₂O, 8/10) to obtain H9-2 (400 mg) as a white solid, with a yield of 33%. LCMS ESI-MS m/z: 705 [M+H]⁺.

Step 3: The compound H9-2 (90 mg, 0.13 mmol) from the previous step and KOAc (75 mg, 0.77 mmol) were dissolved in 1 mL DMSO, and morpholine (56 mg, 0.64 mmol) was added. The mixture was reacted at 120° C. for 3 hours, and then the reaction was stopped. 10 mL of water was added to the reaction solution. The mixture was extracted with dichloromethane, dried over anhydrous sodium sulfate, and concentrated. The crude was separated by HPLC chromatography (Column: Column: YMC-Actus Triart C₁₈ ExRS30*150 mm, 5 m; mobile phase A: water (10 mmol/L NH₄HCO₃+0.05% NH₃·H₂O), mobile phase B: acetonitrile; flow rate: 60 mL/min; retention time: 7.0 min) to obtain H9 (23 mg) as a white solid, with a yield of 25%. LCMS ES-MS m/z: 712 [M+H]⁺.

¹H NMR (300 MHz, DMSO-d₆) (510.49 (s, 2H), 8.09 (dd, J=21.4, 4.3 Hz, 2H), 7.79 (d, J=1.8 Hz, 2H), 7.57 (dq, J=5.7, 2.0 Hz, 1H), 7.34-7.19 (m, 2H), 5.23 (s, 2H), 4.55 (d, J=12.4 Hz, 1H), 3.70-3.60 (i, 4H), 3.48 (d, J=10.6 Hz, 2H), 3.40 (d, J=9.7 Hz, 5H), 3.22 (J=11.9 Hz, 1H), 2.98 (dd, J=14.3, 7.9 Hz, 3H), 2.78 (d, J=11.2 Hz, 1H), 2.60 (d, J=10.9 Hz, 1H), 1.19 (t, J=7.4 Hz, 3H).

Referring to the synthetic route of compound H9, the following target molecules were synthesized using similar raw materials/intermediates.

| | | | LC-MS |
|---|---|---|---|
| Target | | | ESI-MS |
| molecule | Molecular structure | ¹H NMR | m/z: [M + H]⁺ |
| H10 | 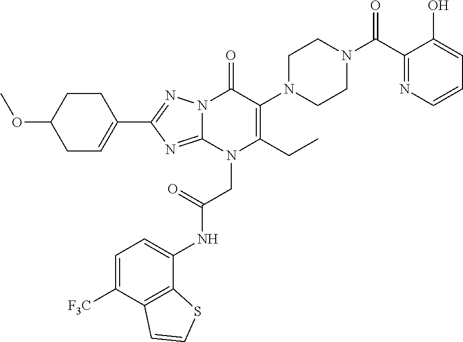 [SMILES: CCc1c(N2CCN(C(=O)c3ncccc3O)CC2)c(=O)n2nc(C3=CCC(OC)CC3)nc2n1CC(=O)Nc1ccc(C(F)(F)F)c2ccsc12] | ¹H NMR: (300 MHz, DMSO-d₆) δ 10.54 (s, 2H), 8.11 (d, J = 5.5 Hz, 1H), 8.06 (dd, J = 3.5, 2.4 Hz, 1H), 7.79 (d, J = 1.7 Hz, 2H), 7.57 (dq, J = 5.6, 1.9 Hz, 1H), 7.32-7.24 (m, 2H), 5.22 (s, 2H), 4.55 (d, J = 12.4 Hz, 1H), 3.78 (dt, J = 13.3, 4.9 Hz, 2H), 3.49 (dt, J = 11.8, 6.1 Hz, 2H), 3.38 (q, J = 4.1 Hz, 2H), 3.25 (s, 3H), 3.17 (ddd, J = 12.9, 9.3, 3.4 Hz, 3H), 2.96 (dt, J = 13.3, 9.1 Hz, 3H), 2.77 (d, J = 11.1 Hz, 1H), 2.60 (d, J = 10.9 Hz, 1H), 1.90-1.79 (m, 2H), 1.41 (dtd, J = 12.7, 8.8, 3.9 Hz, 2H), 1.19 (t, J = 7.5 Hz, 3H). | 740 |
| H11 | 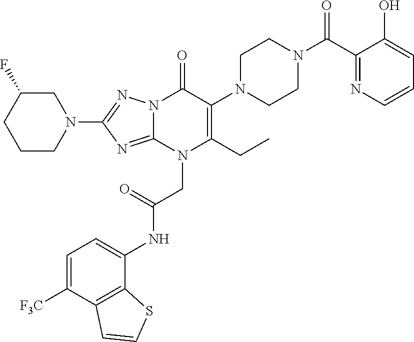 [SMILES: CCc1c(N2CCN(C(=O)c3ncccc3O)CC2)c(=O)n2nc(N3CCC[C@H](F)C3)nc2n1CC(=O)Nc1ccc(C(F)(F)F)c2ccsc12] | ¹H NMR: (300 MHz, DMSO-d₆) δ 10.55 (s, 2H), 8.11 (d, J = 5.6 Hz, 1H), 8.06 (t, J = 3.0 Hz, 1H), 7.79 (s, 2H), 7.57 (d, J = 5.5 Hz, 1H), 7.29 (d, J = 3.0 Hz, 2H), 5.23 (s, 2H), 4.85-4.66 (m, 1H), 4.55 (d, J = 12.4 Hz, 1H), 3.82-3.70 (m, 1H), 3.69-3.58 (m, 1H), 3.52 (dd, J = 26.6, 13.3 Hz, 2H), 3.42 (s, 1H), 3.39 (s, 1H), 3.27 (s, 1H), 3.21 (t, J = 12.0 Hz, 1H), 2.96 (q, J = 11.5, 10.4 Hz, 3H), 2.77 (d, J = 11.3 Hz, 1H), 2.60 (d, J = 11.0 Hz, 1H), 1.82 (qd, J = 23.5, 21.9, 9.1 Hz, 3H), 1.58 - 1.45 (m, 1H), 1.19 (t, J = 7.5 Hz, 3H). | 728 |

### Example 7

The activity of the molecules of the present disclosure on the hydrolysis of double-stranded PP-6TR DNA by WRN helicase was tested.

The working solution and buffer were prepared, and the test compound was dissolved in DMSO followed by a4-fold dilution (with a final concentration of 10 μM as the starting concentration). Then, 0.2 μL of the test compound solution was added to a384-well plate, followed by 10 μL of (2×) WRN enzyme solution. The mixture was incubated in the dark for 30 minutes, after which 10 μL of substrate detection solution containing double-stranded DNA was added to initiate the reaction (DNA length: 19 bp, labeled with TAMRA at the 3′ end and BHQ2 at the 5′ end). The mixture was incubated at room temperature for 60 minutes. The inhibitory activity (IC₅₀) against the WRN enzyme was calculated based on the fluorescence change (Ex530/Em590) between the blank group (DMSO) and the compound group.

$$Calculation formula : Y = lower platform signal + ( upper platform signal - lower platform signal ) \text{} / ( 1 + 1 {0}^{∧} ( ( Log {IC}_{{50}^{-}} X ) \times Hill slope ) ) \\ X : log value of compound concentration \\ Y : inhibition rate ( \% )$$

TABLE 1
Inhibitory activity of compounds against WRN helicase

| | WRN |
|---|---|
| Compound | unwinding/IC₅₀/nM |
| P1 | 3890 |
| P2 | 2.3 |
| P4 | 1.9 |
| P5 | 1.6 |
| P6 | 3.5 |
| H1 | 1.5 |
| H2 | 1.4 |
| H3 | 1.6 |
| H4 | 71 |
| H5 | 295 |
| H6 | 625 |
| H7 | 9.5 |
| H8 | 1.3 |
| H9 | 1.7 |
| H10 | 1.2 |
| H11 | 2.4 |
| H13 | 1.2 |
| H14 | 3.9 |
| H19 | 0.9 |
| H20 | 1.0 |
| H22 | 1.4 |
| A2 | 16 |

### Unwinding=Unwinding

The above results show that the molecules of the present disclosure have excellent inhibitory effects on DNA unwinding by WRN, and are expected to achieve a better tumor inhibition effect by inhibiting the WRN helicase activity.

Notably, H1-H3, H8-Hii, and H22 molecules display significantly superior activity compared to H4-H6 or the reference molecule A2. Specifically, their WRN helicase inhibitory activity is 10- to 50-fold higher than that of the H4 molecule with benzene ring substitution and more than 100-fold higher than that of the fused-ring molecules at other positions (e.g., H5-H6).

### Example 8

The molecules of the present disclosure were tested for their anti-proliferative activity against MSI-H tumor cells.

The proliferation of tumor cells with microsatellite instability MSI-H is sensitive to WRN inhibitors, while the proliferation of microsatellite stable (MSS) tumor cells is insensitive to WRN inhibitors. By testing the activities of the two, it is shown that the molecules of the present disclosure have an inhibitory and synthetic lethal effect on WRN at the cellular level.

The MSI-unstable SW48 colorectal cancer cells were cultured in RPMI 1640 medium containing 10% FBS and 1% penicillin-streptomycin in a 37° C., 5% CO₂ constant temperature incubator. A 40 μL cell suspension was added to each well of a 384-well microplate. Using Echo, 40 nL of compounds at different concentrations was dispensed into each well, followed by incubation in a 37° C., 5% CO₂ constant temperature incubator for 5 days. Then, 40 μL of CTG solution (Promega, Cat No. G7573) was added to each well, and the plate was incubated in the dark in a 37° C., 5% CO₂ constant temperature incubator for 30 minutes. Luminescence was measured using an Envision multi-function microplate reader (Perkin Elmer, Model: Envision 2104). The luminescent signal was proportional to the amount of ATP in the system, and the amount of ATP directly represented the number of living cells in the system.

IC₅₀ value calculation:

$$Y = lower platform signal + ( upper platform signal - lower platform signal ) \text{} / ( 1 + 1 {0}^{∧} ( ( Log {IC}_{50 -} X ) \times Hill slope ) ) \\ X : log value of compound concentration \\ Y : inhibition rate ( \% )$$

TABLE 2.1
2D antiproliferative effects of compounds
on MSI-H colorectal cancer SW48 cell line

| Compound | SW48/IC₅₀/nM | Compound | SW48/IC₅₀/nM |
|---|---|---|---|
| H1 | 55 | H16 | 137 |
| H2 | 131 | H17 | 123 |
| H3 | 216 | H19 | 125 |
| H4 | 2558 | H20 | 75 |
| H7 | 1938 | H21 | 175 |
| H8 | 124 | H22 | 47 |
| H9 | 193 | H23 | >10000 |
| H10 | 268 | H24 | 474 |
| H11 | 1139 | H25 | 125 |
| H12 | 126 | H26 | 137 |
| H13 | 28 | P4 | 135 |
| H14 | 2196 | P5 | 192 |
| H15 | 101 | | |
| A1 | 261 | A2 | 2537 |

N.D. = Not determined

The reference molecule A1 was synthesized according to WO 2022249060, and has a structure of:

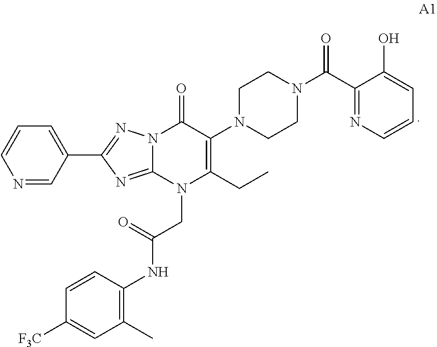 [SMILES: CCc1c(N2CCN(C(=O)c3ncccc3O)CC2)c(=O)n2nc(-c3cccnc3)nc2n1CC(=O)Nc1ccc(C(F)(F)F)cc1C]

The above results demonstrate that the molecules of the present disclosure have excellent anti-proliferative effects against MSI-H tumor cells, and are expected to achieve a better tumor inhibition effect by inhibiting the WRN helicase activity.

Further comparative analysis revealed:

H1-H3, H13, and H22 molecules have significantly superior activity compared to H4, and their activity in inhibiting the proliferation of MSI-H tumor cells is 10 to 40 times higher than that of the H4 molecule with benzene ring substitution. Unexpectedly, fused benzene rings (e.g., benzothiophene or benzofuran) demonstrate markedly higher activity than that of alkylthioether (e.g., methylthio) substitutions.

Hi-H3, H13, and H22 molecules have significantly superior activity compared to A1 or A2, and their activity in inhibiting the proliferation of MSI-H tumor cells is 10 to 90 times higher than that of the A2 molecule with non-heteroaryl substitution.

H1-H3, H13, and H22 molecules have significantly superior activity compared to H14. Surprisingly, some fused benzene rings (e.g., benzothiophene or benzofuran) have significantly higher activity than that of benzothiazole ring substitution.

TABLE 2.2
Antiproliferation value and IC₅₀ calculation
of compound A1 on SW48 cells

| Concentration. | Test 1/ | Test 2/ | Average value/ |
|---|---|---|---|
| [nM] | Inhibition rate | Inhibition rate | Inhibition rate |
| 10000 | 100.9954 | 102.0219 | 101.5087 |
| 2500 | 99.8401 | 101.2267 | 100.5334 |
| 625 | 97.8908 | 96.9311 | 97.4109 |
| 156.25 | −2.1399 | 14.9471 | 6.4036 |
| 39.0625 | −12.6411 | −11.1067 | −11.8739 |
| 9.765625 | 0.0311 | −8.0940 | −4.0314 |
| 2.44140625 | −13.8489 | −8.9023 | −11.3756 |
| 0.610351563 | −2.3700 | −2.2651 | −2.3176 |
| 0.152587891 | −13.1741 | −5.6058 | −9.3899 |
| 0.038146973 | −1.9993 | 1.1876 | −0.4058 |

TABLE 2.3
Antiproliferation value and IC₅₀ calculation
of compound H1 on SW48 cells

| Concentration. | Test 1/ | Test 2/ | Average value/ |
|---|---|---|---|
| [nM] | Inhibition rate | Inhibition rate | Inhibition rate |
| 10000 | 100.7430 | 100.6495 | 100.6963 |
| 2500 | 99.6262 | 100.2164 | 99.9213 |
| 625 | 98.3268 | 98.6771 | 98.5020 |
| 156.25 | 98.4846 | 98.1243 | 98.3044 |
| 39.0625 | 9.3323 | 15.8938 | 12.6131 |
| 9.765625 | −9.6799 | −9.9432 | −9.8115 |
| 2.44140625 | −5.0894 | 2.5108 | −1.2893 |
| 0.610351563 | −8.9821 | −5.5025 | −7.2423 |
| 0.152587891 | −8.9847 | −5.4804 | −7.2326 |
| 0.038146973 | −7.3699 | −8.3885 | −7.8792 |

TABLE 2.4
Antiproliferation value and IC₅₀ calculation
of compound H13 on SW48 cells

| Concentration. | Test 1/ | Test 2/ | Average value/ |
|---|---|---|---|
| [nM] | Inhibition rate | Inhibition rate | Inhibition rate |
| 10000 | 102.5684 | 100.1348 | 101.3516 |
| 2500 | 99.7398 | 99.5299 | 99.6349 |
| 625 | 99.5807 | 99.2305 | 99.4056 |
| 156.25 | 98.6269 | 96.5750 | 97.6010 |
| 39.0625 | 76.2047 | 76.5629 | 76.3838 |
| 9.765625 | −3.9044 | 4.5674 | 0.3315 |
| 2.44140625 | 0.0291 | −6.3647 | −3.1678 |
| 0.610351563 | −3.5648 | 0.7061 | −1.4293 |
| 0.152587891 | 2.4807 | −0.7750 | 0.8529 |
| 0.038146973 | −1.5149 | −1.1038 | −1.3094 |

TABLE 2.5
Antiproliferation value and IC₅₀ calculation
of compound H22 on SW48 cells

| Concentration. | Test 1/ | Test 2/ | Average value/ |
|---|---|---|---|
| [nM] | Inhibition rate | Inhibition rate | Inhibition rate |
| 10000 | 101.5949 | 100.6020 | 101.0985 |
| 2500 | 99.9487 | 99.1673 | 99.5580 |
| 625 | 98.3031 | 97.9254 | 98.1143 |
| 156.25 | 98.3879 | 97.6382 | 98.0130 |
| 39.0625 | 28.4105 | 30.0205 | 29.2155 |
| 9.765625 | −5.4854 | −7.1206 | −6.3030 |
| 2.44140625 | 2.6901 | −3.2971 | −0.3035 |
| 0.610351563 | −1.2705 | −3.6994 | −2.4850 |
| 0.152587891 | −0.1838 | 0.5956 | 0.2059 |
| 0.038146973 | −1.5733 | −6.8114 | −4.1923 |

The above results demonstrate that the control molecule A1 has no inhibitory effect on SW48 cells at a concentration of 156 nM, while the molecules of the present disclosure, such as Hi, H13, H22, etc., still have significant inhibitory effects (>95%) at 156 nM; and even have high inhibitory activity at 39 nM.

### Example 9

The molecules of the present disclosure were tested for their anti-proliferative activity against MSS tumor cells.

The MSS HT-29 colorectal cancer cells were cultured in McCoy's 5A medium containing 10% FBS and 1% penicillin-streptomycin in a 37° C., 5% CO₂ constant temperature incubator. A 40 μL cell suspension was added to each well of a 384-well microplate. Using Echo, 40 nL of compounds at different concentrations was dispensed into each well, followed by incubation in a 37° C., 5% CO₂ constant temperature incubator for 5 days. Then, 40 μL of CTG solution (Promega, Cat No. G7573) was added to each well, and the plate was incubated in the dark in a 37° C., 5% CO₂ constant temperature incubator for 30 minutes. Luminescence was measured using an Envision multi-function microplate reader (Perkin Elmer, Model: Envision 2104). The luminescent signal was proportional to the amount of ATP in the system, and the amount of ATP directly represented the number of living cells in the system.

### IC₅₀ Value Calculation:

$$Y = lower platform signal + ( upper platform signal - lower platform signal ) \text{} / ( 1 + 1 {0}^{∧} ( ( Log {IC}_{50 -} X ) \times Hill slope ) ) \\ X : log value of compound concentration \\ Y : inhibition rate ( \% )$$

TABLE 3
2D antiproliferative effects of compounds
on MSS colorectal cancer HT-29 cell line

| Compound | HT-29/IC₅₀/nM |
|---|---|
| H1 | >10000 |
| H2 | 3497 |
| H3 | >10000 |
| H4 | >10000 |
| H8 | 1480 |
| H9 | >10000 |
| H10 | >10000 |
| H11 | 7408 |
| H12 | >10000 |
| H13 | >10000 |
| H14 | >10000 |
| H15 | >10000 |
| H16 | >10000 |
| H17 | >10000 |
| H18 | >10000 |
| H19 | >10000 |
| H20 | >10000 |
| H21 | >10000 |
| H22 | >10000 |
| H23 | >10000 |
| H24 | N.D. |
| H25 | N.D. |
| H26 | N.D. |

N.D. = Not determined

The above results show that the molecules of the present disclosure have no inhibitory effect on MSS tumor cells, reflecting the high selectivity brought by the selective inhibition of WRN by the molecules of the present disclosure.

### Example 10

The liver microsome stability test of the compounds is as follows:

The compounds of the present disclosure were subjected to a liver microsome stability test. The compounds to be tested were co-incubated with liver microsomes of different species with or without the addition of NADPH. The final concentration of the compounds to be tested in the test system was 1 μM, the final concentration of NADPH was 1 mM, and the final concentration of liver microsomes was 0.5 mg/mL. The concentration of the compound in the incubation supernatant at different time points within 60 minutes was detected and the pharmacokinetic parameters (such as the clearance Clᵢₙₜ) were calculated.

This result indicates that the molecules of the present disclosure have good metabolic stability (especially in the human body).

TABLE 4
In vitro stability test results of compounds
in human or mouse liver microsomes

| | | Human Clᵢₙₜ μL/min/mg |
|---|---|---|
| Compound | Human Clᵢₙₜ/(mL/min/kg) | Protein |
| H1 | 18.5 | 14.8 |
| H2 | 19.7 | 15.7 |
| H3 | 2.7 | 2.2 |
| H9 | 13.0 | 10.3 |
| H12 | 28 | 22 |
| H13 | 7.9 | 6.3 |
| H15 | 2.5 | 2.0 |
| H19 | 0.73 | 0.58 |
| H20 | 25.9 | 20.6 |
| H21 | 6.0 | 4.8 |
| H22 | <6.8 | <5.4 |

N.D. = Not determined

### Example 11

### Membrane Permeability Assessment: Caco-2 Assays

The membrane permeability of the molecule of the present disclosure was evaluated. Samples were analyzed by LC-MS to estimate the apparent permeability coefficient (Pₐₚₚ) of the compounds across Caco-2 cell monolayers, with the apical chamber at pH 6.5 and the basolateral chamber at pH 7.4. Active efflux transport of compounds can be blocked using inhibitors of P-gp efflux transporter, BCRP, and MRP2 (50 μM quinidine, 30 μM benzbromarone, and 20 μM sulfasalazine). The data were used to determine apparent permeability (Papp).

$${P}_{app} = ( {V}_{A} \times {[ drug ]}_{acceptor} ) / ( Area \times Time \times {[ drug ]}_{initial , donor} )$$

where VA is the volume of the receptor pore (in mL), Area is the surface area of the membrane (0.143 cm² for Transwell-96 well permeable scaffolds), and time is the total transport time (in seconds).

$$Efflux Ratio = {P}_{app} {(}_{B - A} ) / {P}_{app} {(}_{A - B} )$$

TABLE 5
Caco-2 membrane permeation data results
of the molecule of the present disclosure

| | Papp(A − B) | Papp(B − A) | |
|---|---|---|---|
| Compound | (10⁻⁶ cm/s) | (10⁻⁶ cm/s) | Efflux |
| H22 | 3.2 | 21 | 6.8 |

The above results indicate that the molecules of the present disclosure have good membrane permeability, and are expected to achieve good tumor inhibition effects with better in vivo pharmacokinetic properties.

### Example 12

### Pharmacokinetic Evaluation Assay in Mice

CD1 female mice were used as test animals and the drug was administered orally/intravenously (oral dosage: 10 mg/kg, intravenous dosage: 2 mg/kg).

Assay protocol: The oral group consisted of 3 mice per group (vehicle: 10% Hβ-CD-pH7.4), and the intravenous group consisted of 3 mice per group. Oral administration: Plasma samples were collected before administration (0 h) and after administration (0.25, 0.5, 1, 2, 4, 8, 24 h). Intravenous administration: Plasma samples were collected before administration (0 h) and after administration (0.083, 0.25, 0.5, 1, 2, 4, 8, 24 h). The blood concentrations in plasma after oral and intravenous administration of mice were determined by LC/MS/MS, respectively. The collected data were calculated using AB Sciex QTRAP 6500 software, and the assay results were as follows:

TABLE 6.1
In vivo PK results of the molecules
of the present disclosure in mice

| Compound | | H1 | H13 | H22 | H15 |
|---|---|---|---|---|---|
| IV (2 mg/kg) | T_{1/2} (h) | 1.6 | 1.2 | 1.4 | 0.3 |
| | C₀ (ng/mL) | 3299 | 2526 | 3095 | 1742 |
| | AUC₀₋₂₄ (h*ng/mL) | 1421 | 722 | 2263 | 655 |
| | Cl (mL/min/kg) | 23.4 | 42.9 | 14.6 | 50.4 |
| PO | T_{1/2} (h) | 1.2 | 1.1 | 3.3 | 1.9 |
| (10 mg/kg) | Tₘₐₓ (h) | 0.5 | 0.5 | 0.5 | 0.25 |
| | Cₘₐₓ(ng/mL) | 4645 | 551 | 3770 | 693 |
| | AUC₀₋₂₄ (h*ng/mL) | 5599 | 943 | 9261 | 1047 |
| | F (%) | 79 | 24 | 84 | 35 |

TABLE 6.2
In vivo PK results of reference molecule A1 compound in mice

| Compound | | A1 |
|---|---|---|
| IV (2 mg/kg) | T_{1/2} (h) | 1.3 |
| | C₀ (ng/mL) | 2520 |
| | AUC₀₋₂₄ (h*ng/mL) | 1186 |
| | Cl (mL/min/kg) | 28.1 |
| PO (10 mg/kg) | T_{1/2} (h) | 1.4 |
| | Tₘₐₓ (h) | 0.5 |
| | Cₘₐₓ(ng/mL) | 584 |
| | AUC₀₋₂₄ (h*ng/mL) | 801 |
| | F (%) | 14 |

The above assay results show that the compounds of the present disclosure have good oral absorption effect, and some molecules (Hi, H22) have higher in vivo exposure than that of the reference molecule A1. Due to the better selectivity, they are expected to bring higher therapeutic effects.

### Example 13

In vivo pharmacodynamic assay in BALB/c nude mice. Details are as follows:

SW 48 (MSI-H) colorectal cancer cells were cultured (L15 medium with 10% fetal bovine serum), and then inoculated into 6-8-week old female BALB/c nude mice (weighing about 20 g), wherein all mice were inoculated subcutaneously. The mice were cultured in an SPF-grade experimental environment, and all mice had free access to commercially certified standard diets. When the average tumor volume of the mice grew to about 150 mm³, the test compound was orally administered daily. The dosage was as follows: blank group vehicle (10% HP-β-CD aqueous solution, pH-7). The dosage of the administration group was 120 mg/kg, 50 mg/kg, or 20 mg/kg, once a day. The tumor volume was measured with a two-dimensional caliper three times a week, and the animals were weighed every day. After 23 consecutive days of administration, the inhibition rate (TGI/100%) was calculated based on the final tumor volume. The tumor volume was calculated using the formula: V=½a*b², where a represents the long diameter of the tumor, and b represents the short diameter of the tumor.

TABLE 7
Antitumor effects of compounds on colorectal
cancer SW48 cells xenografted in nude mice

| | | Tumor volume | Tumor volume | |
|---|---|---|---|---|
| Test drug | Dosage | (mm³)-D 1 | (mm³)-D 21 | TGI |
| Blank group 1 | 0 | 174 | 3026 | 0% |
| H1 | 120 mg/kg, QD | 171 | 28 | 105% |
| Blank group 2 | 0 | 163 | 1966 | 0% |
| H22 | 50 mg/kg, QD | 162 | 89 | 104% |
| A1 | 50 mg/kg, QD | 160 | 1065 | 50% |

The results show that the molecules of the present disclosure have good in vivo efficacy against MSI-H microsatellite unstable tumor cells and have lower toxicity to mice. Compared with the reference molecule A1, the molecules of the present disclosure significantly improve the tumor inhibition effect in vivo.

1. A compound of formula (I), or a pharmaceutically acceptable salt, isotopic variant, tautomer, or stereoisomer thereof: 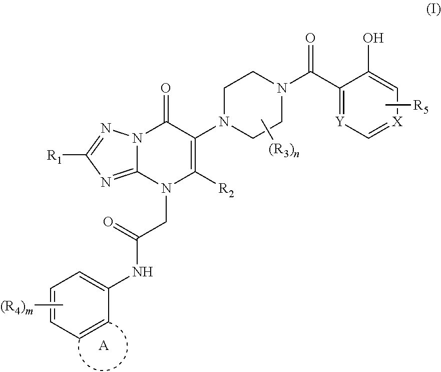
wherein, [symbol]
represents a single bond or a double bond;
X and Y are each independently selected from CH and N, and at least one of X and Y is N;
R₁ is selected from 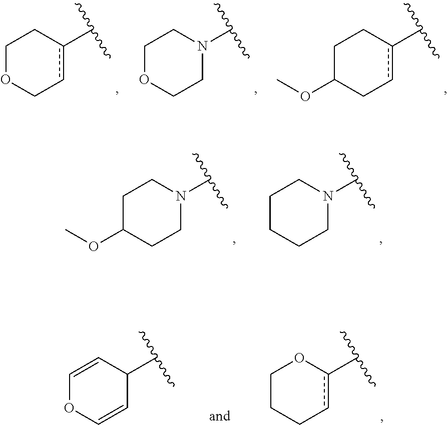
alternatively, R₁ is selected from 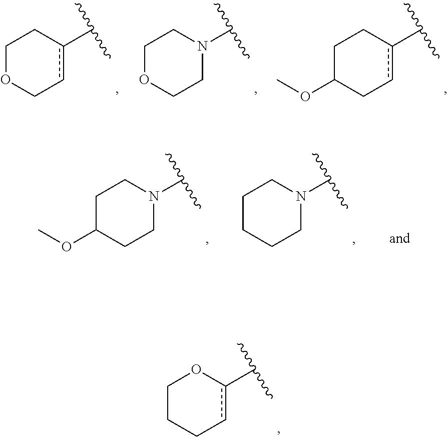
and the R₁ is optionally substituted with 1 or 2 Rₓ;
R₂ is selected from H, C₁₋₆ alkyl, C₁₋₆ haloalkyl, C₁₋₆ alkoxy, C₁₋₆ alkylthio and C₃₋₆ cycloalkyl;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, —SCF₃, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

2. The compound of claim 1, or a pharmaceutically acceptable salt, isotopic variant, tautomer, or stereoisomer thereof, which is a compound of formula (II): 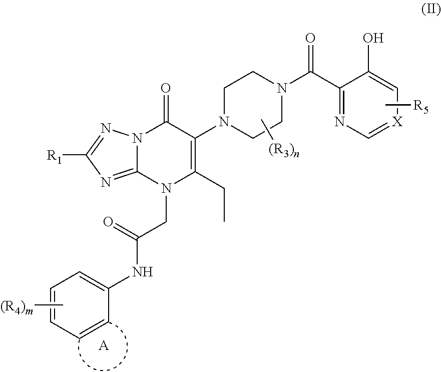
wherein, [symbol]
represents a single bond or a double bond;
X is selected from CH and N;
R₁ is selected from 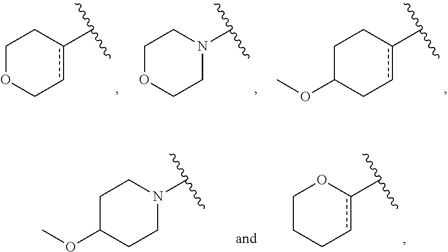
and the R₁ is optionally substituted with 1 or 2 Rₓ;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
Ring A and the benzene ring to which it is connected together form a fused ring, and Ring A is selected from 5- to 6-membered heteroaryl and 5- to 7-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

3. The compound of claim 1, or a pharmaceutically acceptable salt, isotopic variant, tautomer, or stereoisomer thereof, having the following general structure: 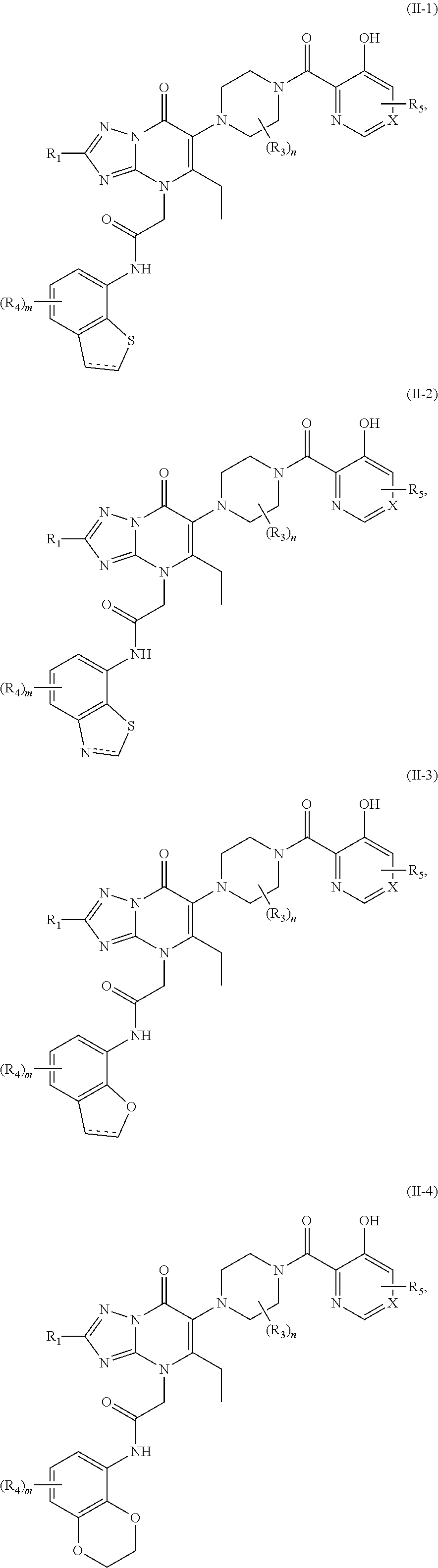 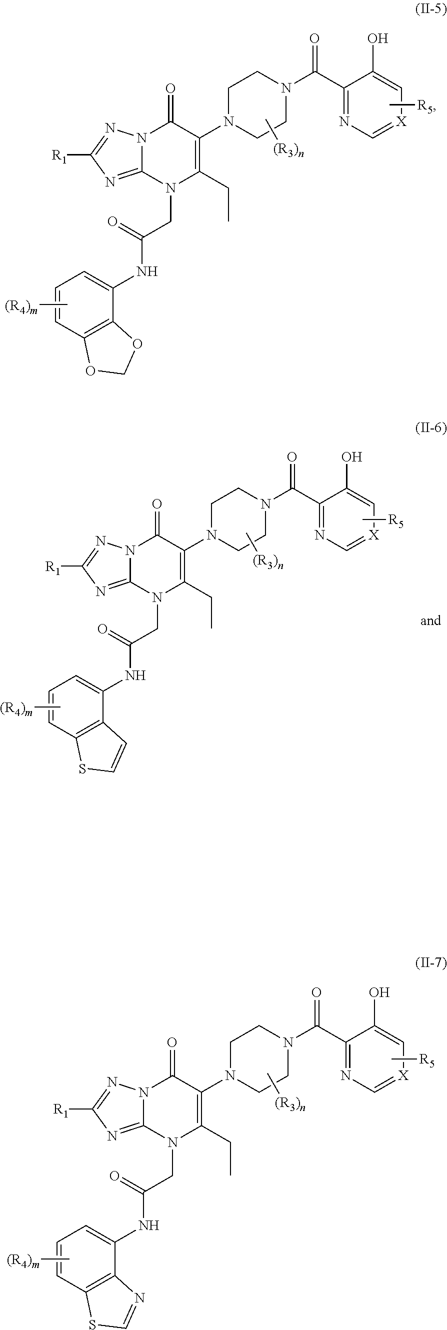
wherein, [symbol]
represents a single bond or a double bond;
X is selected from CH and N;
R₁ is selected from 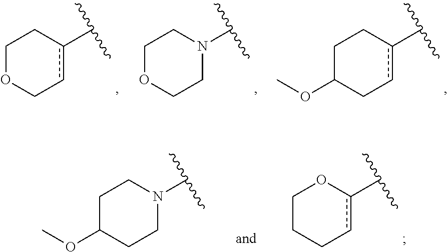
the R₁ is optionally substituted with 1 or 2 Rₓ;
R₃ is independently selected from H, halogen, CN, C₁₋₆ alkyl, C₁₋₆ haloalkyl and C₃₋₆ cycloalkyl; or two R₃ on the same or different carbon atoms are connected to form a 3- to 6-membered spiro or bridged ring;
R₄ is independently selected from H, halogen, CN, SF₅, C₁₋₆ alkyl, C₁₋₆ haloalkyl, C₁₋₆ alkylthio, C₃₋₆ cycloalkyl and 4- to 10-membered heterocyclyl;
R₅ is selected from H, halogen, C₁₋₆ alkyl, C₁₋₆ haloalkyl, C₁₋₆ alkoxy, C₁₋₆ alkylthio and C₃₋₆ cycloalkyl;
Rₓ is selected from H, C₁₋₆ alkyl, C₁₋₆ haloalkyl, C₁₋₆ alkoxy, CN, NH₂, —C(O)Rₐ, —C(O)ORₐ, —(CH₂)ₚ—ORₐ, —P(O)—(Rₐ)₂ and —S(O)₂—Rₐ;
Rₐ is selected from H, C₁₋₆ alkyl and C₃₋₆ cycloalkyl;
m is selected from 0, 1, 2 and 3;
n is selected from 0, 1 and 2;
p is selected from 0, 1 and 2.

4. The compound of claim 3, or a pharmaceutically acceptable salt, isotopic variant, tautomer, or stereoisomer thereof, wherein, [symbol]
represents a single bond or a double bond;
X is selected from CH and N;
R₁ is selected from 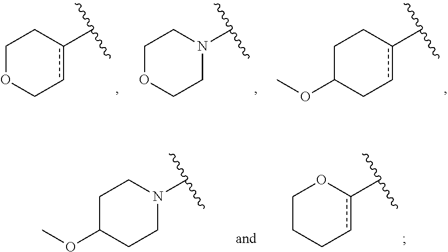
the R₁ is optionally substituted with 1 or 2 Rₓ;
R₃ is independently selected from H, F, CN, methyl, ethyl, trifluoromethyl and cyclopropyl;
or two R₃ on the same carbon atom are connected to form cyclopropyl or cyclobutyl;
R₄ is independently selected from H, F, Cl, Br, CN, SF₅, methyl, ethyl, trifluoromethyl, difluoromethyl, methylthio and cyclopropyl;
R₅ is selected from H, F, Cl, Br, methyl, ethyl, trifluoromethyl, methoxy, methylthio and cyclopropyl;
Rₓ is selected from H, methyl, trifluoromethyl, methoxy, CN and NH₂;
m is selected from 0, 1, 2 and 3;
n is selected from 0, 1 and 2;
p is selected from 0, 1 and 2.

5. The compound of claim 1, or a pharmaceutically acceptable salt, isotopic variant, tautomer, or stereoisomer thereof, which is a compound of formula (III): 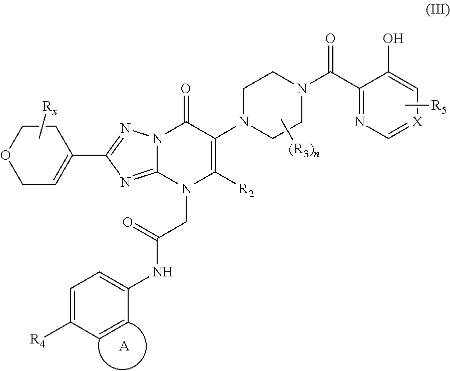
wherein,
X is selected from CH and N;
Ring A is a 5- to 6-membered heteroaryl, wherein the 5- to 6-membered heteroaryl group contains 1 heteroatom selected from O, S and N;
Rₓ is selected from H, C₁₋₆ alkyl, C₁₋₆ haloalkyl and C₁₋₆ alkoxy;
R₂ is selected from H, C₁₋₆ alkyl, C₁₋₆ haloalkyl and C₁₋₆ alkoxy;
R₃ is selected from H, halogen, C₁₋₆ alkyl and C₁₋₆ haloalkyl;
R₄ is selected from H, halogen, C₁₋₆ alkyl and C₁₋₆ haloalkyl;
R₅ is selected from H, halogen, C₁₋₆ alkyl, C₁₋₆ haloalkyl and C₁₋₆ alkoxy;
n is 0, 1 or 2;
alternatively,
X is selected from CH and N;
Ring A is a 5- to 6-membered heteroaryl, wherein the 5- to 6-membered heteroaryl group contains 1 heteroatom selected from O, S and N;
Rₓ is selected from H, C₁₋₆ alkyl and C₁₋₆ haloalkyl;
R₂ is selected from H, C₁₋₆ alkyl and C₁₋₆ haloalkyl;
R₃ is selected from H and C₁₋₆ alkyl;
R₄ is selected from halogen and C₁₋₆ haloalkyl;
R₅ is selected from H, halogen, C₁₋₆ alkyl and C₁₋₆ haloalkyl;
n is 0, 1 or 2;
still alternatively,
X is selected from CH and N;
Ring A is a 5-membered heteroaryl group, wherein the 5-membered heteroaryl contains 1 heteroatom selected from O, S and N;
Rₓ is selected from H and C₁₋₄ alkyl, alternatively H;
R₂ is selected from H and C₁₋₄ alkyl, alternatively CH₂CH₃;
R₃ is selected from H and C₁₋₄ alkyl, such as H and CH₃;
R₄ is selected from halogen and C₁₋₄ haloalkyl, such as Br, Cl and CF₃;
R₅ is selected from H, halogen and C₁₋₄ alkyl, alternatively F, Cl and CH₃;
n is 0, 1 or 2.

6. The compound of claim 5, or a pharmaceutically acceptable salt, isotopic variant, tautomer, or stereoisomer thereof, which is a compound of formula (III-1) or formula (III-2): 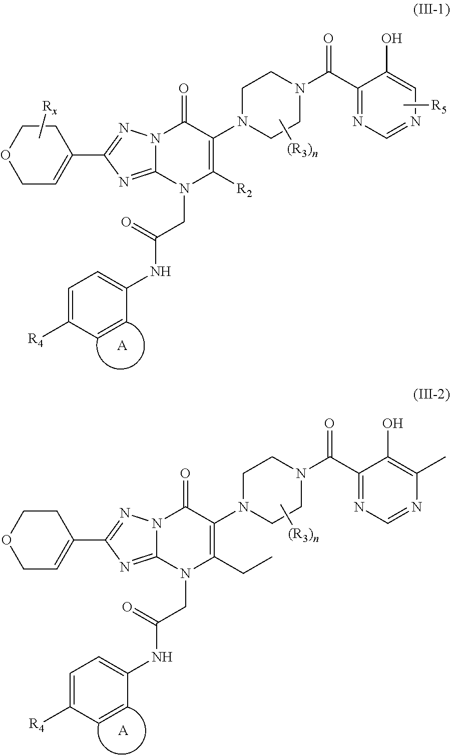
wherein,
the variables are as defined in claim 5.

7. The compound of claim 1, or a pharmaceutically acceptable salt, isotopic variant, tautomer, or stereoisomer thereof, which is a compound of formula (IV) or formula (V): 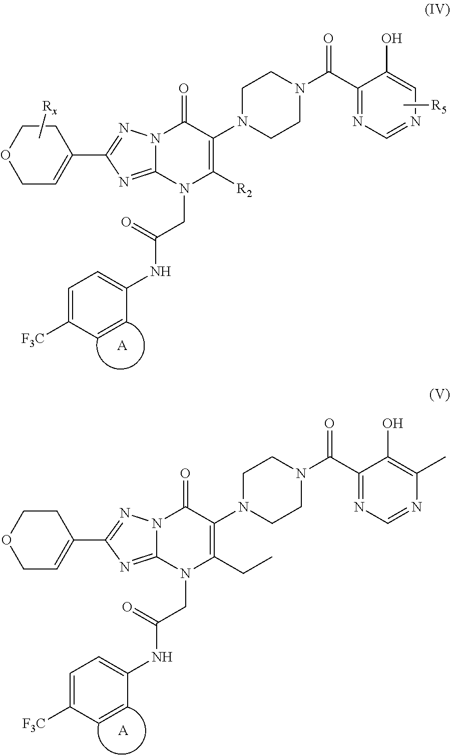
wherein,
Ring A is a 5- to 6-membered heteroaryl, wherein the 5- to 6-membered heteroaryl contains 1 heteroatom selected from O, S and N;
Rₓ is selected from H, C₁₋₆ alkyl, C₁₋₆ haloalkyl and C₁₋₆ alkoxy;
R₂ is selected from H, C₁₋₆ alkyl, C₁₋₆ haloalkyl and C₁₋₆ alkoxy;
R₅ is selected from H, halogen, C₁₋₆ alkyl, C₁₋₆ haloalkyl and C₁₋₆ alkoxy;
alternatively,
Ring A is a 5-membered heteroaryl group, wherein the 5-membered heteroaryl contains 1 heteroatom selected from O, S and N;
Rₓ is selected from H, C₁₋₆ alkyl and C₁₋₆ haloalkyl;
R₂ is selected from H, C₁₋₆ alkyl and C₁₋₆ haloalkyl;
R₅ is selected from H, halogen, C₁₋₆ alkyl and C₁₋₆ haloalkyl;
still alternatively,
Ring A is a 5-membered heteroaryl group, wherein the 5-membered heteroaryl contains 1 heteroatom selected from O, S and N;
Rₓ is selected from H and C₁₋₄ alkyl, alternatively H;
R₂ is selected from H and C₁₋₄ alkyl, alternatively CH₂CH₃;
R₅ is selected from H and C₁₋₄ alkyl, alternatively CH₃.

8. A compound, or a tautomer, stereoisomer or pharmaceutically acceptable salt thereof, wherein the compound is selected from:

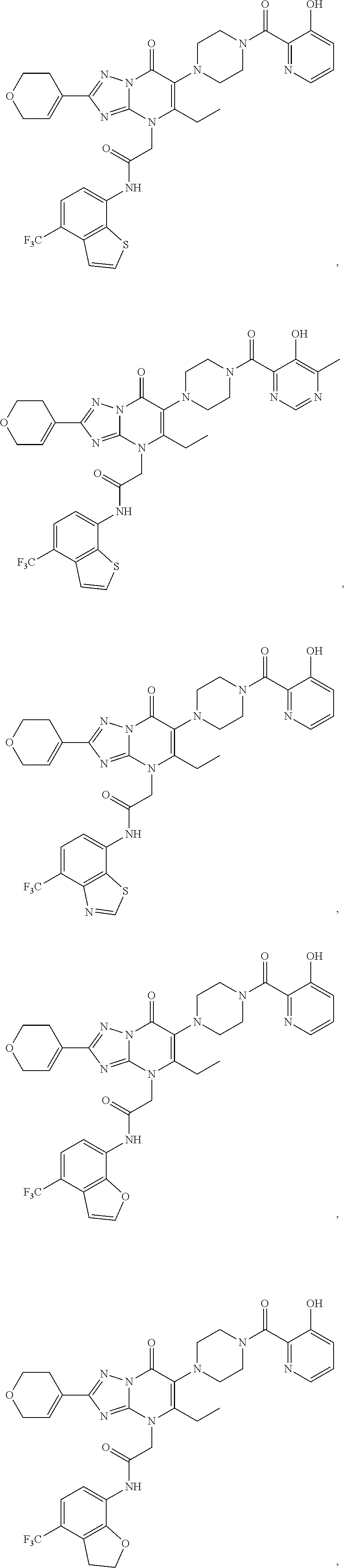 [SMILES: CCc1c(N2CCN(C(=O)c3ncccc3O)CC2)c(=O)n2nc(C3=CCOCC3)nc2n1CC(=O)Nc1ccc(C(F)(F)F)c2ccsc12 ; CCc1c(N2CCN(C(=O)c3ncnc(C)c3O)CC2)c(=O)n2nc(C3=CCOCC3)nc2n1CC(=O)Nc1ccc(C(F)(F)F)c2ccsc12 ; CCc1c(N2CCN(C(=O)c3ncccc3O)CC2)c(=O)n2nc(C3=CCOCC3)nc2n1CC(=O)Nc1ccc(C(F)(F)F)c2ncsc12 ; CCc1c(N2CCN(C(=O)c3ncccc3O)CC2)c(=O)n2nc(C3=CCOCC3)nc2n1CC(=O)Nc1ccc(C(F)(F)F)c2ccoc12 ; CCc1c(N2CCN(C(=O)c3ncccc3O)CC2)c(=O)n2nc(C3=CCOCC3)nc2n1CC(=O)Nc1ccc(C(F)(F)F)c2c1OCC2]

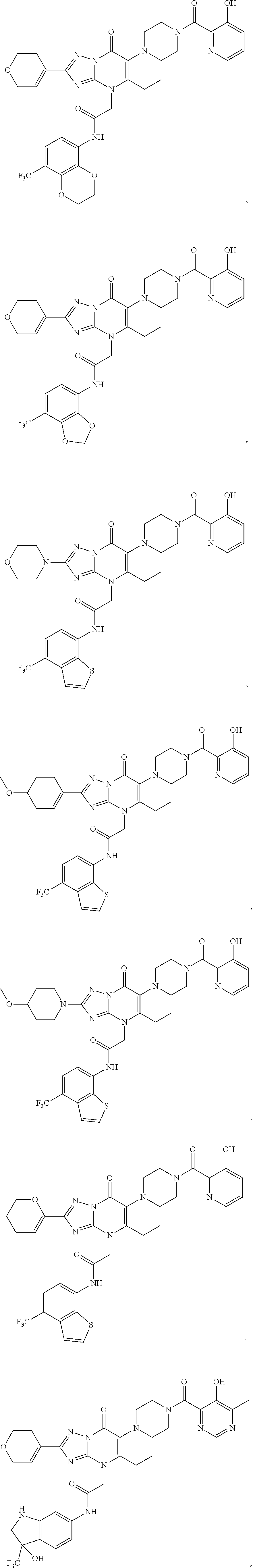

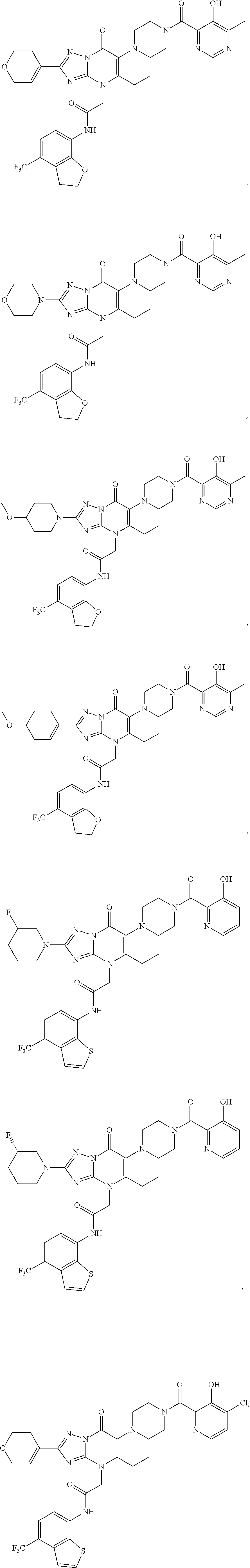

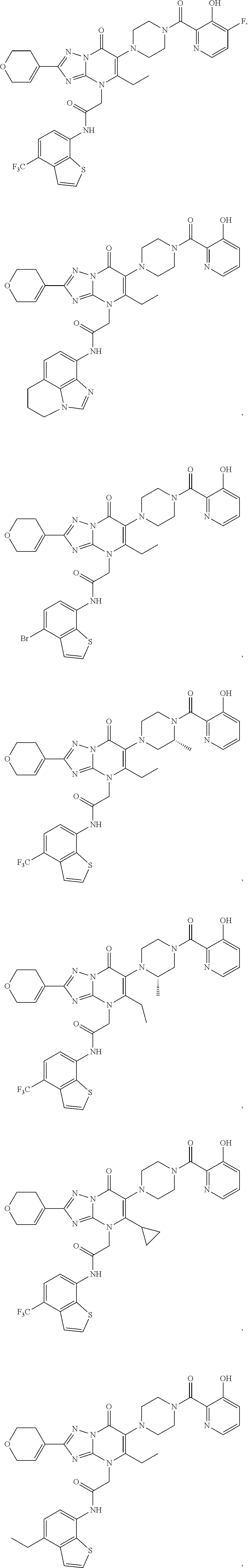

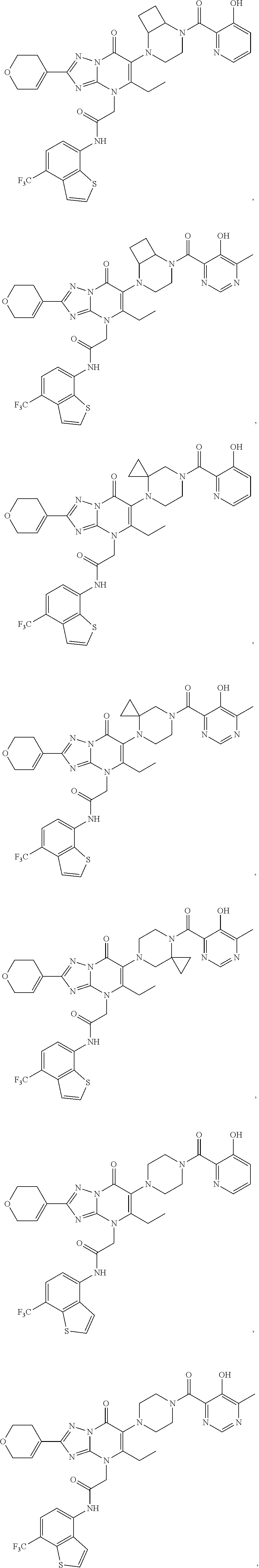

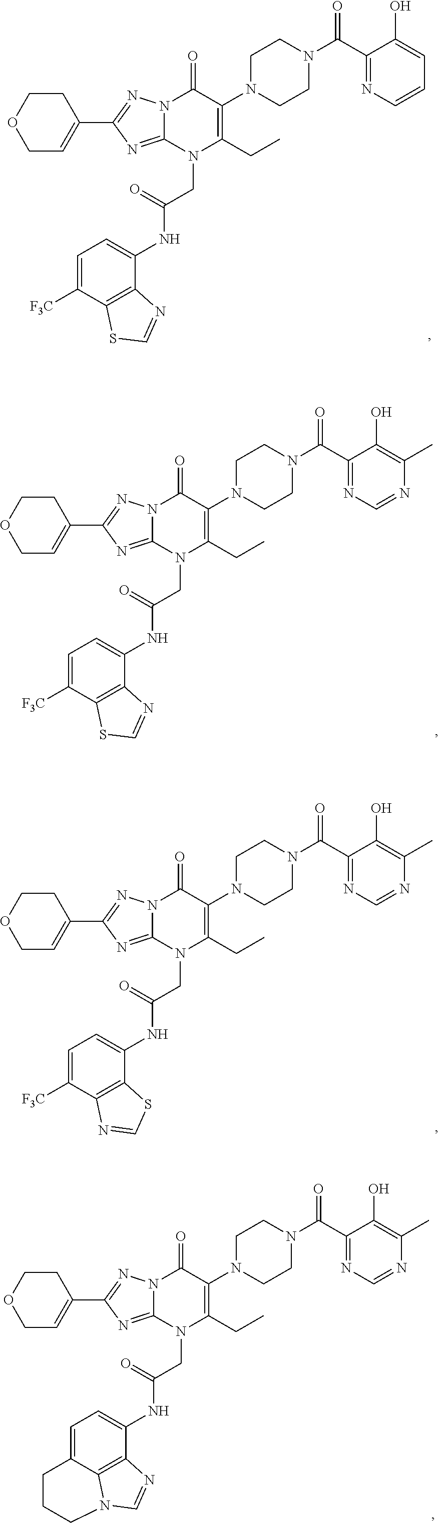 [SMILES: CCc1c(N2CCN(C(=O)c3ncccc3O)CC2)c(=O)n2nc(C3=CCOCC3)nc2n1CC(=O)Nc1ccc(C(F)(F)F)c2scnc12 ; CCc1c(N2CCN(C(=O)c3ncnc(C)c3O)CC2)c(=O)n2nc(C3=CCOCC3)nc2n1CC(=O)Nc1ccc(C(F)(F)F)c2scnc12 ; CCc1c(N2CCN(C(=O)c3ncnc(C)c3O)CC2)c(=O)n2nc(C3=CCOCC3)nc2n1CC(=O)Nc1ccc(C(F)(F)F)c2ncsc12 ; CCc1c(N2CCN(C(=O)c3ncnc(C)c3O)CC2)c(=O)n2nc(C3=CCOCC3)nc2n1CC(=O)Nc1ccc2c3c1ncn3CCC2]

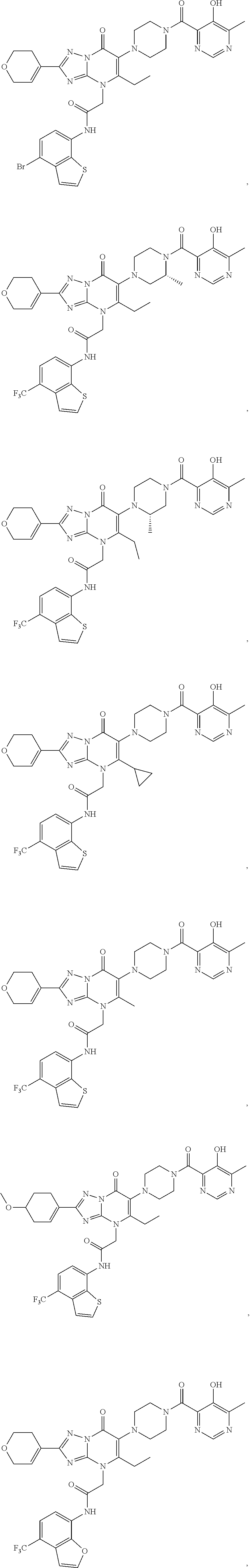

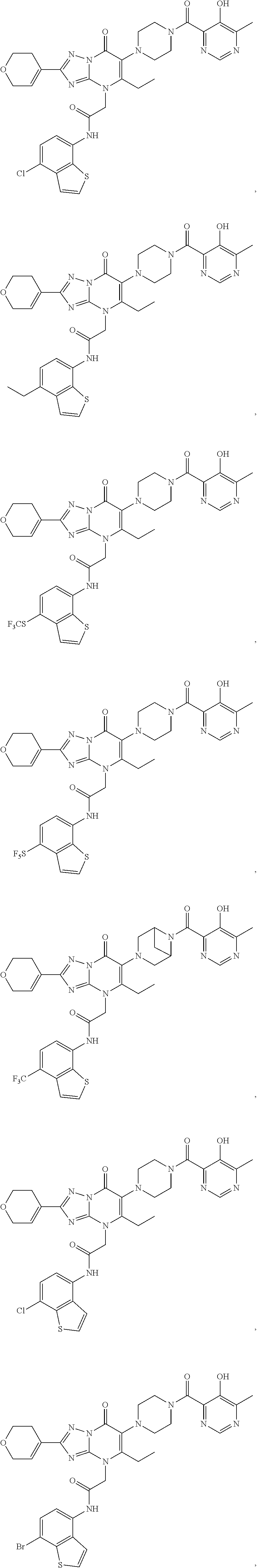

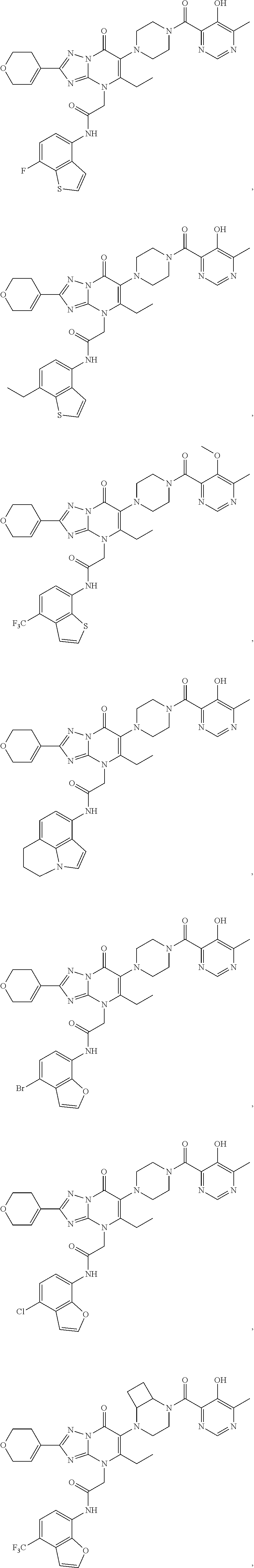

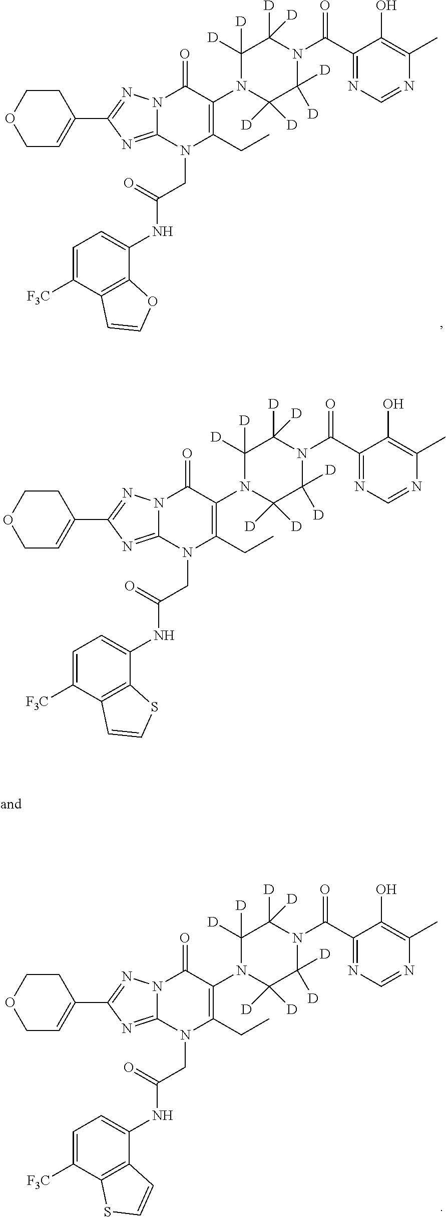 [SMILES: [2H]C1([2H])N(C(=O)c2ncnc(C)c2O)C([2H])([2H])C([2H])([2H])N(c2c(CC)n(CC(=O)Nc3ccc(C(F)(F)F)c4ccoc34)c3nc(C4=CCOCC4)nn3c2=O)C1([2H])[2H] ; [2H]C1([2H])N(C(=O)c2ncnc(C)c2O)C([2H])([2H])C([2H])([2H])N(c2c(CC)n(CC(=O)Nc3ccc(C(F)(F)F)c4ccsc34)c3nc(C4=CCOCC4)nn3c2=O)C1([2H])[2H] ; [2H]C1([2H])N(C(=O)c2ncnc(C)c2O)C([2H])([2H])C([2H])([2H])N(c2c(CC)n(CC(=O)Nc3ccc(C(F)(F)F)c4sccc34)c3nc(C4=CCOCC4)nn3c2=O)C1([2H])[2H]]

9. A pharmaceutical composition comprising a compound according to claim 1, or a pharmaceutically acceptable salt, isotopic variant, tautomer, or stereoisomer thereof.

10. (canceled)

11. A method for treating and/or preventing a WRN-mediated disease in a subject, the method comprising administering to the subject the compound according to claim 1 or a pharmaceutically acceptable salt, isotopic variant, tautomer, or stereoisomer thereof.

12. (canceled)

13. The method of claim 11, wherein the WRN-mediated disease is cancer, and the cancer is selected from the group consisting of: acoustic neuroma, adenocarcinoma, adrenal gland cancer, anal cancer; angiosarcoma (e.g., lymphangiosarcoma, lymphangioendotheliosarcoma, hemangiosarcoma), appendix cancer, benign monoclonal gammopathy, bile duct cancer, bladder cancer, brain cancer (e.g., meningioma, glioma, e.g., astrocytoma, oligodendroglioma, medulloblastoma), bronchial cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer))), hematopoietic cancer (e.g., leukemia, such as acute lymphocytic leukemia (ALL) (e.g., B-cell ALL, T-cell ALL), acute myeloid leukemia (AML) (e.g., B-cell AML, T-cell AML), chronic myeloid leukemia (CML) (e.g., B-cell CML, T-cell CML), chronic lymphocytic leukemia (CLL) (e.g., B-cell CLL, T-cell CLL), follicular lymphoma, chronic lymphocytic leukemia/small lymphocytic lymphoma (CLL/SLL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, lymphoplasmacytic lymphoma, hairy cell leukemia (HCL), immunoblastic large cell lymphoma, precursor B lymphoblastic lymphoma, and primary central nervous system (CNS) lymphomas; and T-cell non-Hodgkin's lymphomas, such as precursor T-lymphoblastic lymphoma/leukemia, peripheral T-cell lymphomas (such as cutaneous T-cell lymphomas (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma (MM), heavy chain disease (e.g., alpha chain disease, gamma chain disease, mu chain disease), hemangioblastoma, inflammatory myofibroblastic tumor, immunocytic amyloidosis, kidney cancer (e.g., nephroblastoma, renal cell carcinoma), liver cancer (e.g., hepatocellular cancer, malignant hepatoma), lung cancer (such as bronchogenic carcinoma, small cell lung cancer (SCLC), non-small cell lung cancer (NSCLC), lung adenocarcinoma, leiomyosarcoma (LMS), mastocytosis (such as systemic mastocytosis), myelodysplastic syndrome (MDS), mesothelioma, myeloproliferative diseases (MPD) (such as polycythemia vera (PV), essential thrombocythemia (ET), agnogenic myeloid metaplasia (AMM), chronic idiopathic myelofibrosis, chronic myeloid leukemia (CML), chronic neutrophilic leukemia (CNL), hypereosinophilic syndrome (HES), neuroblastoma, neurofibromas (such as neurofibromatosis type 1 or type 2, schwannomatosis), neuroendocrine cancer (such as gastroenteropancreatic neuroendocrine tumor (GEP-NET), carcinoid tumor), osteosarcoma, ovarian cancer (such as cystadenocarcinoma, ovarian embryonal carcinoma, ovarian adenocarcinoma), papillary adenocarcinoma, penile cancer.

14. The compound of claim 1, or a pharmaceutically acceptable salt, isotopic variant, tautomer, or stereoisomer thereof, having the following general structure: 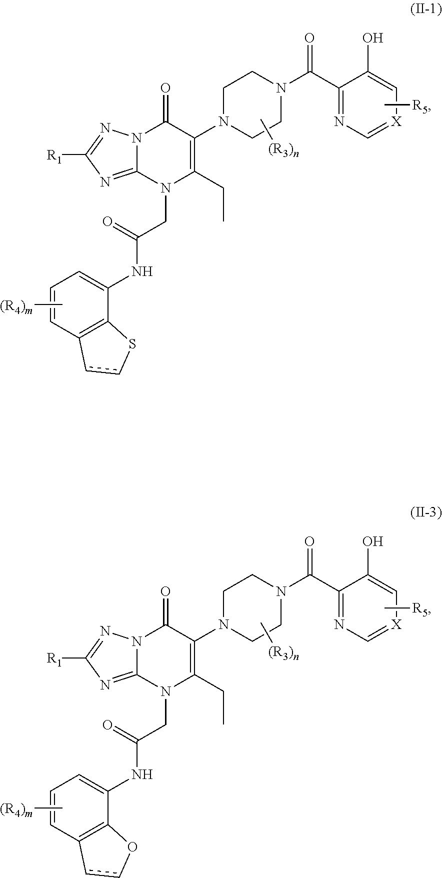
wherein, [symbol]
represents a single bond or a double bond; alternatively, [symbol] represents a double bond;
X is selected from CH and N;
R₁ is selected from 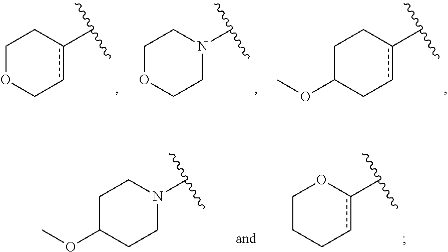
alternatively is 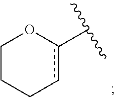
the R₁ is optionally substituted with 1 or 2 Rₓ;
R₃ is independently selected from H, halogen, C₁₋₆ alkyl, and C₁₋₆ haloalkyl;
R₄ is independently selected from H, halogen, C₁₋₆ alkyl, and C₁₋₆ haloalkyl;
R₅ is selected from H, halogen, C₁₋₆ alkyl, and C₁₋₆ haloalkyl;
Rₓ is selected from H, C₁₋₆ alkyl, and C₁₋₆ haloalkyl;
m is selected from 0, 1, 2 and 3;
n is selected from 0, 1 and 2.

15. The compound of claim 1, or a pharmaceutically acceptable salt, isotopic variant, tautomer, or stereoisomer thereof, wherein the compound is:

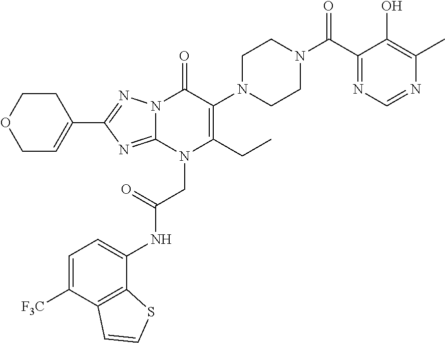 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ccsc12]

16. The compound of claim 1, or a pharmaceutically acceptable salt, isotopic variant, tautomer, or stereoisomer thereof, wherein the compound is: 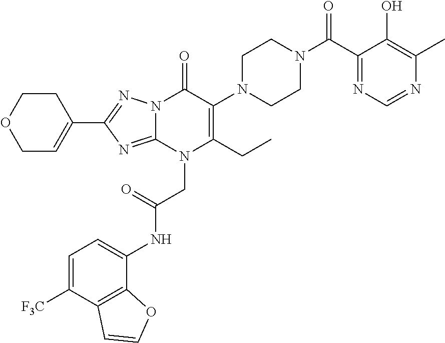 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c2ccoc12]
